SEQUENCE LISTING (85 sequences)

SEQ ID NO: 1 - DNA, 36 bp, Artificial
  Forwared primer of SL335 H Xba1 Kozak
gatcaactctagagccaccatggagtggtcctgggt

SEQ ID NO: 2 - DNA, 27 bp, Artificial
  Reverse primer of GS vector GSAP linker
gtgctacctggggcaggggctgacccg

SEQ ID NO: 3 - DNA, 27 bp, Artificial
  Forward primer of GS vector GSAP linker
cgggtcagcccctgccccaggtagcac

SEQ ID NO: 4 - DNA, 47 bp, Artificial
  Reverse primer of huIL18BP Not1, Bbs1
cgcgaagacgcttttagagcggccgcgtctacctacccttgctgctg

SEQ ID NO: 5 - DNA, 55 bp, Artificial
  Forward primer of huIL18BP only
ggctgagcggggtggaggggacacctgtgtcccagaccacaacagccgctacagc

SEQ ID NO: 6 - DNA, 90 bp, Artificial
  Reverse primer of huIL18BP his8 BbsI NotI
atcggcggccgcgaagacgcttttagatcagtggtggtggtgatggtggtggtgccccccttgctgctgtgggctagaatggctacttgg

SEQ ID NO: 7 - protein, 164 aa, Artificial
  IL-18BP
TPVSQTTTAATASVRSTKDPCPSQPPVFPAAKQCPALEVTWPEVEVPLNGTLSLSCVACSRFPNFSILYWLGNGSFIEHLPGRLWEGSTSRERGSTGTQLCKALVLEQLTPALHSTNFSCVLVDPEQVVQRHVVLAQLWAGLRATLPPTQEALPSSHSSPQQQG

SEQ ID NO: 8 - DNA, 492 bp, Artificial
  IL-18BP
acacctgtgtcccagaccacaacagccgctacagcttcagtgagatctactaaagatccttgtccttcccagccccctgtttttcccgctgccaaacaatgtcctgctcttgaagttacatggccagaggtcgaggttccacttaatggaacactcagcctctcttgtgtggcatgtagtcgctttcccaatttctcaatactttattggctcggaaatggaagtttcatcgagcatttgcctgggcggctttgggaaggcagcactagcagagaacgcggttcaacagggacacagctctgtaaagccctggtgctggagcagttgactccagcccttcactctactaacttctcctgcgttctcgtggaccctgagcaagtggtccagaggcatgtagttcttgcacagctttgggccggactgcgagcaactctgccacccactcaggaagccctcccaagtagccattctagcccacagcagcaaggg

SEQ ID NO: 9 - DNA, 492 bp, Artificial
  IL-18BP
accccagtgtcccaaaccaccaccgcggctaccgcctccgtccggtccactaaggatccttgcccgagccagccgccggtgttccctgccgcgaaacagtgccccgcactggaagtgacctggcccgaagtggaagtccccctcaatggtaccctgagcctctcatgcgtggcatgctcaaggttcccgaacttctcgatcctctactggctgggaaacgggtcgttcatcgagcatctgcccggacgcctctgggagggatccactagccgcgagcgcgggagcaccggcacccagctgtgcaaggccttggtgcttgagcagctgactccggccctgcactctacgaacttctcctgcgtgttggtggaccctgaacaagtggtgcagagacacgtcgtgctggcccagctctgggccgggctgcgggccacactgccgcccactcaagaagccctgccaagctcccactcgagcccacagcagcagggt

SEQ ID NO: 10 - protein, 223 aa, Artificial
  SL335H
QVQLVQSGGGPVKPGGSLRLSCAASGFMFRAYSMNWVRQAPGKGLEWVSSISSSGRYIHYADSVKGRFTISRDNAKNSLYLQMNSLRAEDTAVYYCARETVMAGKALDYWGQGTLVTVSSASTKGPSVFPLAPSSKSTSEGTAALGCLVKDYFPEPVTVSWNSGALTSGVHTFPAVLQSSGLYSLSSVVTVPSSSLGTQTYICNVNHKPSNTKVDKKVEPKSS

SEQ ID NO: 11 - DNA, 669 bp, Artificial
  SL335H
caagtgcaattggtgcagtcaggtggcggcccagtaaaacccggtgggtctctgagattgagctgcgctgcatccggatttatgttccgtgcctacagcatgaattgggtgcggcaagctcctggaaagggactcgaatgggtttccagtattagcagttctgggaggtatatacattatgctgactcagtgaaagggaggttcacaattagccgggacaatgccaaaaactctctctatctgcagatgaacagccttcgcgccgaagataccgccgtttattactgcgctcgggagactgtgatggccggtaaggctcttgactattggggacagggaactttggtgactgtttcatctgcctctacaaagggacccagcgtttttccattggcacctagtagcaagtccacatctgaaggtacagctgctttggggtgtttggtgaaagactacttccccgaaccagtgacagtttcatggaactccggcgctttgactagtggagtgcataccttcccagctgttcttcagagtagtgggctttatagtttgagtagcgtcgtgactgtcccatcatcctctctcggcacacaaacctatatctgcaatgtaaaccataagccatcaaacaccaaagtagacaagaaagttgagccaaagtccagc

SEQ ID NO: 12 - DNA, 669 bp, Artificial
  SL335H
caagtccagcttgtgcagtccggcggtggaccagtgaagcctgggggatcactgcgcctctcctgtgccgcttcggggttcatgttccgggcatactcgatgaactgggttagacaggctcccggaaagggcctggaatgggtgtccagtatctcaagctcgggccgctacattcattatgcggacagcgtgaagggcagattcaccattagccgggacaatgccaagaactccctgtacttgcaaatgaactccctgagggccgaggatactgcggtgtactattgtgctcgggagactgtgatggccggaaaggccctggactactggggccagggcacccttgtgaccgtgtcctccgcctcgaccaagggaccgtcggtgtttcctctggcgccgtcctcgaagtcaacctccgagggaaccgccgccctgggttgcctcgtgaaagactacttccctgaacccgtgactgtgtcctggaacagcggtgccctgacctccggggtgcatactttccctgcggtgctgcagtcgtccggactctactccctgtcatccgtcgtgaccgtgccttcctcctccctgggaacccagacctacatctgcaacgtgaaccacaagccctccaacaccaaggtcgacaagaaagtcgagcccaagtccagc

SEQ ID NO: 13 - protein, 215 aa, Artificial
  SL335L
DIVLTQSPGTLSLSPGETATLSCRASQSVGSNLAWYQQKPGQAPRLLIYGASTGATGVPARFSGSRSGTDFTLTITSLQPEDFATYYCQQYYSFLAKTFGQGTQLEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGES

SEQ ID NO: 14 - DNA, 645 bp, Artificial
  SL335L
gacatcgtcctgacccagagccccgggacactgagcctgtcccccggggagacagccacactgagctgcagggccagccagtccgtcggctccaacctggcctggtaccagcagaagcccgggcaggcccccaggctgctgatttacggcgccagcacaggcgccacaggggtgcccgccaggtttagcggcagccggagcggcacagattttacactgacaatcacatccctgcagcccgaggattttgccacatactactgccagcagtactactccttcctggccaagacattcggccagggcacccagctggagatcaagcggaccgtggccgcccccagcgtgtttattttcccccccagcgatgagcagctgaagtccggcaccgcctccgtcgtgtgcctgctgaacaacttctacccccgggaggccaaggtccagtggaaggtcgataacgccctgcagagcgggaactcccaggagtccgtgaccgagcaggactccaaggacagcacctactccctgtccaacaccctgaccctgagcaaggccgactacgagaagcacaaggtctacgcctgcgaggtgacccaccagggcctgtcctcccccgtgaccaagagctttaaccggggggagtcc

SEQ ID NO: 15 - DNA, 645 bp, Artificial
  SL335L
gatatcgtgctgactcagtcgcctggtaccctttccctgtcacccggagaaactgccaccctgagctgtagagcgagccagtccgtgggctccaacctggcctggtatcaacagaagcctgggcaggcccctcgcctgttgatctacggcgccagcactggtgccaccggcgtgccagctcggttctccggctcccggtcggggactgacttcaccctcaccattacgagcctgcagcccgaagatttcgcgacctactactgccaacagtactactcattcctggctaagactttcggacaaggcacccagctcgagatcaagcggaccgtggcagcaccgtccgtgttcattttcccgccgtccgacgagcaacttaagtccggaaccgcctctgtcgtgtgcctcctcaacaacttctacccgcgcgaggccaaggtccagtggaaggtcgacaacgcgctgcagtccggaaattcacaggaaagcgtgaccgaacaggactccaaggattcgacctactccctgtccaacactctgaccctgtcgaaagccgactacgagaagcacaaagtgtacgcctgcgaagtgacacatcagggactcagctcgcccgtgaccaagagcttcaacaggggagagtcg

SEQ ID NO: 16 - protein, 8 aa, Artificial
  Linker 1
GSAPAPGS

SEQ ID NO: 17 - DNA, 24 bp, Artificial
  Linker 1
gggtcagcccctgccccaggtagc

SEQ ID NO: 18 - DNA, 24 bp, Artificial
  Linker 1
ggctcggccccggcaccgggcagc

SEQ ID NO: 19 - protein, 395 aa, Artificial
  SL335H + peptide linker + IL-18BP
QVQLVQSGGGPVKPGGSLRLSCAASGFMFRAYSMNWVRQAPGKGLEWVSSISSSGRYIHYADSVKGRFTISRDNAKNSLYLQMNSLRAEDTAVYYCARETVMAGKALDYWGQGTLVTVSSASTKGPSVFPLAPSSKSTSEGTAALGCLVKDYFPEPVTVSWNSGALTSGVHTFPAVLQSSGLYSLSSVVTVPSSSLGTQTYICNVNHKPSNTKVDKKVEPKSSGSAPAPGSTPVSQTTTAATASVRSTKDPCPSQPPVFPAAKQCPALEVTWPEVEVPLNGTLSLSCVACSRFPNFSILYWLGNGSFIEHLPGRLWEGSTSRERGSTGTQLCKALVLEQLTPALHSTNFSCVLVDPEQVVQRHVVLAQLWAGLRATLPPTQEALPSSHSSPQQQG

SEQ ID NO: 20 - DNA, 1185 bp, Artificial
  SL335H + peptide linker + IL-18BP
caagtgcaattggtgcagtcaggtggcggcccagtaaaacccggtgggtctctgagattgagctgcgctgcatccggatttatgttccgtgcctacagcatgaattgggtgcggcaagctcctggaaagggactcgaatgggtttccagtattagcagttctgggaggtatatacattatgctgactcagtgaaagggaggttcacaattagccgggacaatgccaaaaactctctctatctgcagatgaacagccttcgcgccgaagataccgccgtttattactgcgctcgggagactgtgatggccggtaaggctcttgactattggggacagggaactttggtgactgtttcatctgcctctacaaagggacccagcgtttttccattggcacctagtagcaagtccacatctgaaggtacagctgctttggggtgtttggtgaaagactacttccccgaaccagtgacagtttcatggaactccggcgctttgactagtggagtgcataccttcccagctgttcttcagagtagtgggctttatagtttgagtagcgtcgtgactgtcccatcatcctctctcggcacacaaacctatatctgcaatgtaaaccataagccatcaaacaccaaagtagacaagaaagttgagccaaagtccagcgggtcagcccctgccccaggtagcacacctgtgtcccagaccacaacagccgctacagcttcagtgagatctactaaagatccttgtccttcccagccccctgtttttcccgctgccaaacaatgtcctgctcttgaagttacatggccagaggtcgaggttccacttaatggaacactcagcctctcttgtgtggcatgtagtcgctttcccaatttctcaatactttattggctcggaaatggaagtttcatcgagcatttgcctgggcggctttgggaaggcagcactagcagagaacgcggttcaacagggacacagctctgtaaagccctggtgctggagcagttgactccagcccttcactctactaacttctcctgcgttctcgtggaccctgagcaagtggtccagaggcatgtagttcttgcacagctttgggccggactgcgagcaactctgccacccactcaggaagccctcccaagtagccattctagcccacagcagcaaggg

SEQ ID NO: 21 - DNA, 1185 bp, Artificial
  SL335H + peptide linker + IL-18BP
caagtccagcttgtgcagtccggcggtggaccagtgaagcctgggggatcactgcgcctctcctgtgccgcttcggggttcatgttccgggcatactcgatgaactgggttagacaggctcccggaaagggcctggaatgggtgtccagtatctcaagctcgggccgctacattcattatgcggacagcgtgaagggcagattcaccattagccgggacaatgccaagaactccctgtacttgcaaatgaactccctgagggccgaggatactgcggtgtactattgtgctcgggagactgtgatggccggaaaggccctggactactggggccagggcacccttgtgaccgtgtcctccgcctcgaccaagggaccgtcggtgtttcctctggcgccgtcctcgaagtcaacctccgagggaaccgccgccctgggttgcctcgtgaaagactacttccctgaacccgtgactgtgtcctggaacagcggtgccctgacctccggggtgcatactttccctgcggtgctgcagtcgtccggactctactccctgtcatccgtcgtgaccgtgccttcctcctccctgggaacccagacctacatctgcaacgtgaaccacaagccctccaacaccaaggtcgacaagaaagtcgagcccaagtccagcggctcggccccggcaccgggcagcaccccagtgtcccaaaccaccaccgcggctaccgcctccgtccggtccactaaggatccttgcccgagccagccgccggtgttccctgccgcgaaacagtgccccgcactggaagtgacctggcccgaagtggaagtccccctcaatggtaccctgagcctctcatgcgtggcatgctcaaggttcccgaacttctcgatcctctactggctgggaaacgggtcgttcatcgagcatctgcccggacgcctctgggagggatccactagccgcgagcgcgggagcaccggcacccagctgtgcaaggccttggtgcttgagcagctgactccggccctgcactctacgaacttctcctgcgtgttggtggaccctgaacaagtggtgcagagacacgtcgtgctggcccagctctgggccgggctgcgggccacactgccgcccactcaagaagccctgccaagctcccactcgagcccacagcagcagggt

SEQ ID NO: 22 - protein, 5 aa, Artificial Sequence
  Heavy chain variable domain CDR1
SYGIS

SEQ ID NO: 23 - protein, 17 aa, Artificial Sequence
  Heavy chain variable domain CDR2
WINTYSGGTKYAQKFQG

SEQ ID NO: 24 - protein, 15 aa, Artificial Sequence
  Heavy chain variable domain CDR3
LGHCQRGICSDALDT

SEQ ID NO: 25 - protein, 17 aa, Artificial Sequence
  Heavy chain variable domain CDR2
RINTYNGNTGYAQRLQG

SEQ ID NO: 26 - protein, 5 aa, Artificial Sequence
  Heavy chain variable domain CDR1
NYGIH

SEQ ID NO: 27 - protein, 17 aa, Artificial Sequence
  Heavy chain variable domain CDR2
SISYDGSNKYYADSVKG

SEQ ID NO: 28 - protein, 16 aa, Artificial Sequence
  Heavy chain variable domain CDR3
DVHYYGSGSYYNAFDI

SEQ ID NO: 29 - protein, 5 aa, Artificial Sequence
  Heavy chain variable domain CDR1
SYAMS

SEQ ID NO: 30 - protein, 17 aa, Artificial Sequence
  Heavy chain variable domain CDR2
VISHDGGFQYYADSVKG

SEQ ID NO: 31 - protein, 11 aa, Artificial Sequence
  Heavy chain variable domain CDR3
AGWLRQYGMDV

SEQ ID NO: 32 - protein, 5 aa, Artificial Sequence
  Heavy chain variable domain CDR1
AYWIA

SEQ ID NO: 33 - protein, 17 aa, Artificial Sequence
  Heavy chain variable domain CDR2
MIWPPDADARYSPSFQG

SEQ ID NO: 34 - protein, 8 aa, Artificial Sequence
  Heavy chain variable domain CDR3
LYSGSYSP

SEQ ID NO: 35 - protein, 5 aa, Artificial Sequence
  Heavy chain variable domain CDR1
AYSMN

SEQ ID NO: 36 - protein, 17 aa, Artificial Sequence
  Heavy chain variable domain CDR2
SISSSGRYIHYADSVKG

SEQ ID NO: 37 - protein, 11 aa, Artificial Sequence
  Heavy chain variable domain CDR3
ETVMAGKALDY

SEQ ID NO: 38 - protein, 11 aa, Artificial Sequence
  Light chain variable domain CDR1
RASQSISRYLN

SEQ ID NO: 39 - protein, 7 aa, Artificial Sequence
  Light chain variable domain CDR2
GASRLES

SEQ ID NO: 40 - protein, 9 aa, Artificial Sequence
  Light chain variable domain CDR3
QQSDSVPVT

SEQ ID NO: 41 - protein, 11 aa, Artificial Sequence
  Light chain variable domain CDR1
RASQSISSYLN

SEQ ID NO: 42 - protein, 7 aa, Artificial Sequence
  Light chain variable domain CDR2
AASSLQS

SEQ ID NO: 43 - protein, 10 aa, Artificial Sequence
  Light chain variable domain CDR3
QQSYSTPPYT

SEQ ID NO: 44 - protein, 11 aa, Artificial Sequence
  Light chain variable domain CDR1
RASQSIFNYVA

SEQ ID NO: 45 - protein, 7 aa, Artificial Sequence
  Light chain variable domain CDR2
DASNRAT

SEQ ID NO: 46 - protein, 11 aa, Artificial Sequence
  Light chain variable domain CDR3
QQRSKWPPTWT

SEQ ID NO: 47 - protein, 12 aa, Artificial Sequence
  Light chain variable domain CDR1
RASETVSSRQLA

SEQ ID NO: 48 - protein, 7 aa, Artificial Sequence
  Light chain variable domain CDR2
GASSRAT

SEQ ID NO: 49 - protein, 9 aa, Artificial Sequence
  Light chain variable domain CDR3
QQYGSSPRT

SEQ ID NO: 50 - protein, 12 aa, Artificial Sequence
  Light chain variable domain CDR1
RASQSVSSSSLA

SEQ ID NO: 51 - protein, 9 aa, Artificial Sequence
  Light chain variable domain CDR3
QKYSSYPLT

SEQ ID NO: 52 - protein, 11 aa, Artificial Sequence
  Light chain variable domain CDR1
RASQSVGSNLA

SEQ ID NO: 53 - protein, 7 aa, Artificial Sequence
  Light chain variable domain CDR2
GASTGAT

SEQ ID NO: 54 - protein, 10 aa, Artificial Sequence
  Light chain variable domain CDR3
QQYYSFLAKT

SEQ ID NO: 55 - protein, 124 aa, Artificial Sequence
  Heavy chain variable domain 1
QVQLLQSGAEVKKPGASVKVSCKASGYTFTSYGISWVRQAPGQGLEWVGWINTYSGGTKYAQKFQGRVTMTRDTSISTVYMELSGLKSDDTAVYYCARLGHCQRGICSDALDTWGQGTLVTVSS

SEQ ID NO: 56 - protein, 124 aa, Artificial Sequence
  Heavy chain variable domain 2
EVQLLQSGAEVKEPGASVKVSCKASGYTFSSYGISWVRQAPGQGLEWVGRINTYNGNTGYAQRLQGRVTMTTDTSTSIAYMEVRSLRSDDTAVYYCARLGHCQRGICSDALDTWGQGTMVTVSS

SEQ ID NO: 57 - protein, 125 aa, Artificial Sequence
  Heavy chain variable domain 3
QVQLVQSGGGVVQTGGSLRLSCAASGFTFRNYGIHWVRQAPGKGLEWVASISYDGSNKYYADSVKGRFTISRDNSRNTVHVQMDSLRGGDTAVYYCARDVHYYGSGSYYNAFDIWGQGTLVTVSS

SEQ ID NO: 58 - protein, 120 aa, Artificial Sequence
  Heavy chain variable domain 4
QVQLVQSGGGLVQPGGSLRLSCAASGFTFSSYAMSWVRQAPGKGLEWLSVISHDGGFQYYADSVKGRFTVSRDNSKNTLYLQMNSLRAEDTAVYYCARAGWLRQYGMDVWGQGTLVTVSS

SEQ ID NO: 59 - protein, 117 aa, Artificial Sequence
  Heavy chain variable domain 5
EVQLVQSGTEVKKPGESLKISCKISGYSFTAYWIAWVRQMPGKGLEWMGMIWPPDADARYSPSFQGQVTFSVDKSISTAYLQWHSLKTSDTAVYYCARLYSGSYSPWGQGTLVTVSS

SEQ ID NO: 60 - protein, 120 aa, Artificial Sequence
  Heavy chain variable domain 6
QVQLVQSGGGPVKPGGSLRLSCAASGFMFRAYSMNWVRQAPGKGLEWVSSISSSGRYIHYADSVKGRFTISRDNAKNSLYLQMNSLRAEDTAVYYCARETVMAGKALDYWGQGTLVTVSS

SEQ ID NO: 61 - protein, 108 aa, Artificial Sequence
  Light chain variable domain 1
ELVLTQSPSSLSASVGDRVTITCRASQSISRYLNWYQQKPGKAPKLLIYGASRLESGVPSRFSGSGSGTDFTLTISSLQPEDFATYYCQQSDSVPVTFGQGTRLEIKR

SEQ ID NO: 62 - protein, 109 aa, Artificial Sequence
  Light chain variable domain 2
DIVLTQSPSSLSASVGDRVTITCRASQSISSYLNWYQQKPGKAPKLLIYAASSLQSGVPSRFSGSGSGTDFTLTISSLQPEDFATYYCQQSYSTPPYTFGQGTKLEIKR

SEQ ID NO: 63 - protein, 110 aa, Artificial Sequence
  Light chain variable domain 3
ELVLTQSPGTLSLSPGERATLSCRASQSIFNYVAWYQQKPGQAPRLLIYDASNRATGIPARFSGSGSGTDFTLTISSLEPEDFAVYYCQQRSKWPPTWTFGQGTRVDIKR

SEQ ID NO: 64 - protein, 109 aa, Artificial Sequence
  Light chain variable domain 4
ELVLTQSPGTLSLSPGERATLSCRASETVSSRQLAWYQQKPGQAPRLLIYGASSRATGIPDRFSGSGSGTDFTLTISRLEPEDSAVFYCQQYGSSPRTFGGGTKLEIKR

SEQ ID NO: 65 - protein, 109 aa, Artificial Sequence
  Light chain variable domain 5
ELVLTQSPGTLSLSPGERATLSCRASQSVSSSSLAWYQQKPGQAPRLLIYGASSRATGIPDRFSGSGSGTDFTLTISSLQPEDAATYYCQKYSSYPLTFGQGTKLEIKR

SEQ ID NO: 66 - protein, 109 aa, Artificial Sequence
  Light chain variable domain 6
ELVLTQSPGTLSLSPGETATLSCRASQSVGSNLAWYQQKPGQAPRLLIYGASTGATGVPARFSGSRSGTDFTLTITSLQPEDFATYYCQQYYSFLAKTFGQGTQLEIKR

SEQ ID NO: 67 - protein, 109 aa, Artificial Sequence
  Light chain variable domain 7
DIVLTQSPGTLSLSPGETATLSCRASQSVGSNLAWYQQKPGQAPRLLIYGASTGATGVPARFSGSRSGTDFTLTITSLQPEDFATYYCQQYYSFLAKTFGQGTQLEIKR

SEQ ID NO: 68 - protein, 103 aa, Artificial Sequence
  Heavy chain constant domain
ASTKGPSVFPLAPSSKSTSEGTAALGCLVKDYFPEPVTVSWNSGALTSGVHTFPAVLQSSGLYSLSSVVTVPSSSLGTQTYICNVNHKPSNTKVDKKVEPKSS

SEQ ID NO: 69 - protein, 107 aa, Artificial Sequence
  Light chain constant domain
RTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGES

SEQ ID NO: 70 - protein, 15 aa, Artificial Sequence
  Linker 2
GSAGSAPAPAGSGEF

SEQ ID NO: 71 - protein, 10 aa, Artificial Sequence
  Linker 3
GGGGSGGGGS

SEQ ID NO: 72 - protein, 5 aa, Artificial Sequence
  Linker 4
GGGGS

SEQ ID NO: 73 - protein, 5 aa, Artificial Sequence
  Linker 5
SGGGG

SEQ ID NO: 74 - protein, 5 aa, Artificial Sequence
  Linker 6
SRSSG

SEQ ID NO: 75 - protein, 5 aa, Artificial Sequence
  Linker 7
SGSSC

SEQ ID NO: 76 - protein, 12 aa, Artificial Sequence
  Linker 8
GKSSGSGSESKS

SEQ ID NO: 77 - protein, 6 aa, Artificial Sequence
  Linker 9
RPPPPC

SEQ ID NO: 78 - protein, 7 aa, Artificial Sequence
  Linker 10
SSPPPPC

SEQ ID NO: 79 - protein, 14 aa, Artificial Sequence
  Linker 11
GSTSGSGKSSEGKG

SEQ ID NO: 80 - protein, 18 aa, Artificial Sequence
  Linker 12
GSTSGSGKSSEGSGSTKG

SEQ ID NO: 81 - protein, 18 aa, Artificial Sequence
  Linker 13
GSTSGSGKPGSGEGSTKG

SEQ ID NO: 82 - protein, 14 aa, Artificial Sequence
  Linker 14
EGKSSGSGSESKEF

SEQ ID NO: 83 - protein, 15 aa, Artificial Sequence
  Linker 15
GSAGSAPAPAGSGEF

SEQ ID NO: 84 - protein, 10 aa, Artificial Sequence
  Linker 16
GGGGSGGGGS

SEQ ID NO: 85 - protein, 8 aa, Artificial Sequence
  Histidine tag
HHHHHHHH

(19) United States
(12) Patent Application Publication
(10) Pub. No.: US 2023/0357340 A1
(43) Pub. Date: Nov. 9, 2023

(54) Recombinant Fusion Proteins Comprising Interleukin-18-Binding Protein and Antigen Binding Fragment to Serum Albumin, and Compositions and Uses Thereof

(71) Applicant: Aprilbio Co., Ltd., Chuncheon-si, Gangwon-do (KR)

(72) Inventor: Sang Hoon CHA, Chuncheon-si, Gangwon-do (KR)

(21) Appl. No.: 18/246,423

(22) PCT Filed: Sep. 29, 2021
(86) PCT No.: PCT/IB2021/058964

§ 371 Date: Mar. 23, 2023

(30) Foreign Application Priority Data
Sep. 29, 2020 (KR) 10-2020-0127395

(51) Int. Cl. C07K 14/47; C07K 16/18; A61P 37/06

(52) U.S. Cl. CPC C07K 14/4703; C07K 16/18; A61P 37/06; C07K 2317/92; C07K 2319/21; C07K 2317/55; C07K 2317/76; C07K 2319/31; C07K 2317/94; A61K 2039/505

(57) ABSTRACT

Provided are recombinant fusion proteins comprising an interleukin-18-binding protein and an antigen binding fragment against serum albumin and uses thereof. The recombinant fusion proteins have an improved administration cycle due to an increase in the half-life in the body. Further, the recombinant fusion proteins have low immunogenicity and do not cause side effects in vivo, and therefore, can be effectively used for the treatment of various cancers and immune diseases and conditions.

### CROSS-REFERENCE TO RELATED APPLICATIONS

This application claims priority to KR Appl. No. 10-2020-0127395, filed Sep. 29, 2020, the disclosure of which is incorporated herein by reference in its entirety.

### REFERENCE TO SEQUENCE LISTING SUBMITTED ELECTRONICALLY

The content of the electronically submitted sequence listing in ASCII text file (Name: 2662-0005WO01_Sequence_Listing_ST25.txt; Size: 46 KB; and Date of Creation: Sep. 29, 2021) filed with the application is incorporated herein by reference in its entirety.

### TECHNICAL FIELD

The present disclosure relates to recombinant proteins comprising an interleukin-18-binding protein and an antigen binding fragment that binds to serum albumin, nucleic acid molecules encoding the recombinant proteins, vectors, cells, compositions, and uses thereof.

### BACKGROUND

Autoimmune diseases are caused by autoimmunity due to abnormality in the body's immune system and cause the immune system to incorrectly react to normal chemicals and some cells in the body. The human immune system basically recognizes microorganisms invading the human body and cancer cells as foreign antigens and normally attacks and removes them but does not attack its own cells due to its self-tolerance. However, when the self-tolerance of the immune system is destroyed, the human body continuously destroys its own cells, causing inflammation and immune responses while autoreactive T cells, in response to own cells (or autoantigens), are activated and autoantibodies are generated.

Interleukin-18 (IL-18) is a pro-inflammatory cytokine belonging to the interleukin-1 family and is also known as interferon-gamma inducing factor. Particularly, in the blood of a patient with an immune disease, the concentration of IL-18 is increased, and the concentration of an interleukin-18-binding protein, which is an antagonist of IL-18, is lower than that of IL-18. For this reason, it is necessary to reduce the concentration of interleukin-18 in the blood. Clinical trials conducted on a small number of patients reported that when biological agents targeting inflammatory cytokines such as interleukin-1, interleukin-10, interleukin-6, TNF, etc. were applied to therapy, they exhibit clinical effects in autoimmune diseases. As biological agents may cause anti-drug antibody (ADA), especially in autoimmune diseases, new biological structures can offer alternative options to patients who have ADA against the existing biological agents High levels of IL-18 are also associated with poor prognosis in multiple myeloma (MM) patients (Nakamura, K. et al., Cancer Cell. 2018 Apr. 9; 33(4):634-648.e5). Accordingly, there is a need to develop anti-inflammatory and cancer therapeutic agents capable of increasing administration convenience and efficiency for patients by reducing the dosage and frequency of administration while minimizing side effects.

### SUMMARY OF THE INVENTION

Disclosed herein are recombinant fusion proteins including an interleukin-18-binding protein and an antigen binding fragment against serum albumin.

Also disclosed herein are pharmaceutical compositions for preventing or treating immune diseases, the pharmaceutical compositions including the recombinant fusion protein as an active ingredient.

Also disclosed herein are pharmaceutical compositions for preventing or treating cancer, the pharmaceutical compositions including the recombinant fusion protein as an active ingredient.

Disclosed herein are recombinant fusion proteins comprising an interleukin-18-binding protein (IL-18BP) and an antigen binding fragment (Fab) against serum albumin.

The fusion proteins can further comprise a linker that links the IL-18BP to the Fab. In some embodiments, the linker links the IL-18BP to a C-terminus of the heavy chain constant domain, an N-terminus of the heavy chain variable domain, a C-terminus of the light chain constant domain, and/or an N-terminus of the light chain variable domain of the Fab. In some embodiments, the linker links the IL-18BP to a C-terminus of the heavy chain constant domain. In some embodiments, the linker comprises 1 to 50 amino acids. In some embodiments, the linker comprises an amino acid sequence of any one of SEQ ID NOS:16 and 70-84.

In some embodiments, in the fusion proteins disclosed herein, the heavy chain and the light chain of the Fab are bound by a noncovalent bond.

The Fab can comprise
a heavy chain comprising a heavy chain variable domain comprising
(1) a heavy chain complementarity determining domain 1 (CDR1) comprising the amino acid sequence of SYGIS (SEQ ID NO:22),
   a heavy chain complementarity determining domain 2 (CDR2) comprising the amino acid sequence of WINTYSGGTKYAQKFQG (SEQ ID NO:23), and
   a heavy chain complementarity determining domain 3 (CDR3) comprising the amino acid sequence of LGHCQRGICSDALDT (SEQ ID NO:24);
(2) a heavy chain CDR1 comprising the amino acid sequence of SYGIS (SEQ ID NO:22),
   a heavy chain CDR2 comprising the amino acid sequence of RINTYNGNTGYAQRLQG (SEQ ID NO:25), and
   a heavy chain CDR3 comprising the amino acid sequence of LGHCQRGICSDALDT (SEQ ID NO:24);
(3) a heavy chain CDR1 comprising the amino acid sequence of NYGIH (SEQ ID NO:26),
   a heavy chain CDR2 comprising the amino acid sequence of SISYDGSNKYYADSVKG (SEQ ID NO:27), and
   a heavy chain CDR3 comprising the amino acid sequence of DVHYYGSGSYYNAFDI (SEQ ID NO:28);
(4) a heavy chain CDR1 comprising the amino acid sequence of SYAMS (SEQ ID NO:29),
   a heavy chain CDR2 comprising the amino acid sequence of VISHDGGFQYYADSVKG (SEQ ID NO:30), and
   a heavy chain CDR3 comprising the amino acid sequence of AGWLRQYGMDV (SEQ ID NO:31);
(5) a heavy chain CDRlcomprising the amino acid sequence of AYWIA (SEQ ID NO:32),
   a heavy chain CDR2 comprising the amino acid sequence of MIWPPDADARYSPSFQG (SEQ ID NO:33), and
   a heavy chain CDR3 comprising the amino acid sequence of LYSGSYSP (SEQ ID NO:34); or
(6) a heavy chain CDR1 comprising the amino acid sequence of AYSMN (SEQ ID NO:35),
   a heavy chain CDR2 comprising the amino acid sequence of SISSSGRYIHYADSVKG (SEQ ID NO:36), and
   a heavy chain CDR3 comprising the amino acid sequence of ETVMAGKALDY (SEQ ID NO:37); and
a light chain comprising a light chain variable domain comprising
(7) a light chain CDR1 comprising the amino acid sequence of RASQSISRYLN (SEQ ID NO:38),
   a light chain CDR2 comprising the amino acid sequence of GASRLES (SEQ ID NO:39), and
   a light chain CDR3 comprising the amino acid sequence of QQSDSVPVT (SEQ ID NO:40);
(8) a light chain CDR1 comprising the amino acid sequence of RASQSISSYLN (SEQ ID NO:41),
   a light chain CDR2 comprising the amino acid sequence of AASSLQS (SEQ ID NO:42), and
   a light chain CDR3 comprising the amino acid sequence of QQSYSTPPYT (SEQ ID NO:43);
(9) a light chain CDR1 comprising the amino acid sequence of RASQSIFNYVA (SEQ ID NO:44),
   a light chain CDR2 comprising the amino acid sequence of DASNRAT (SEQ ID NO:45), and
   a light chain CDR3 comprising the amino acid sequence of QQRSKWPPTWT (SEQ ID NO:46);
(10) a light chain CDR1 comprising the amino acid sequence of RASETVSSRQLA (SEQ ID NO:47),
   a light chain CDR2 comprising the amino acid sequence of GASSRAT (SEQ ID NO:48), and
   a light chain CDR3 comprising the amino acid sequence of QQYGSSPRT (SEQ ID NO:49);
(11) a light chain CDR1 comprising the amino acid sequence ofRASQSVSSSSLA (SEQ ID NO:50),
   a light chain CDR2 comprising the amino acid sequence of GASSRAT (SEQ ID NO:48), and
   a light chain CDR3 comprising the amino acid sequence of QKYSSYPLT (SEQ ID NO:51); or
(12) a light chain CDR1 comprising the amino acid sequence of RASQSVGSNLA (SEQ ID NO:52),
   a light chain CDR2 comprising the amino acid sequence of GASTGAT (SEQ ID NO:53), and
   a light chain CDR3 comprising the amino acid sequence of QQYYSFLAKT (SEQ ID NO:54).

In some embodiments, in the fusion proteins disclosed herein, the heavy chain variable domain comprises a heavy chain CDR1 comprising the amino acid sequence of SEQ ID NO:35, a heavy chain CDR2 comprising the amino acid sequence of SEQ ID NO:36, and a heavy chain CDR3 comprising the amino acid sequence of SEQ ID NO:37; and the light chain variable domain comprises a light chain CDR1 comprising the amino acid sequence of SEQ ID NO:52, a light chain CDR2 comprising the amino acid sequence of SEQ ID NO:53, and a light chain CDR3 comprising the amino acid sequence of SEQ ID NO:54.

In some embodiments, the heavy chain variable domain comprises an amino acid sequence having at least 90% identity to SEQ ID NO:55, 56, 57, 58, 59, or 60. In some embodiments, the light chain variable domain comprises an amino acid sequence having at least 90% identity to SEQ ID NO:61, 62, 63, 64, 65, 66, or 67. In some embodiments, the heavy chain variable domain comprises an amino acid sequence of SEQ ID NO:55, 56, 57, 58, 59, or 60, and the light chain variable domain comprises an amino acid sequence of SEQ ID NO:61, 62, 63, 64, 65, 66, or 67. In some embodiments, the heavy chain constant domain comprises an amino acid sequence having at least 90% identity to SEQ ID NO:68. In some embodiments, the light chain constant domain comprises an amino acid sequence having at least 90% identity to SEQ ID NO:69.

The IL-18-binding protein can comprise an amino acid sequence having at least 90% identity to SEQ ID NO:7. In some embodiments, the IL-18-binding protein comprises an amino acid sequence of SEQ ID NO:7.

In some embodiments, the heavy chain of the Fab comprises an amino acid sequence of SEQ ID NO:19. In some embodiments, the fusion protein comprises an amino acid sequence of SEQ ID NO:13 and an amino acid sequence of SEQ ID NO:19.

Also disclosed herein are nucleic acid molecules encoding any of the recombinant fusion proteins disclosed herein.

Further disclosed herein are expression vectors comprising any of the nucleic acid molecules disclosed herein.

Disclosed herein are cells transformed with any of the expression vectors disclosed herein.

Disclosed herein are compositions comprising any of the recombinant fusion proteins disclosed herein. Also disclosed herein are pharmaceutical compositions comprising any of the compositions disclosed herein and a pharmaceutically acceptable excipient. Also disclosed are kits comprising any of the compositions disclosed herein and a label comprising instructions for a use.

Disclosed herein are methods of treating an immune disease in a subject in need thereof, comprising administering an effective amount of the pharmaceutical composition of claim 23 to the subject. In some embodiments, the immune disease is an inflammatory disease or autoimmune disease. In some embodiments, the inflammatory disease is atopic dermatitis, psoriasis, dermatitis, allergy, arthritis, rhinitis, otitis media, sore throat, tonsillitis, cystitis, nephritis, pelvic inflammation, Crohn's disease, ulcerative colitis, ankylosing spondylitis, systemic lupus erythematosus (SLE), asthma, edema, delayed allergy (type IV allergy), transplant rejection, graft-versus-host disease, autoimmune encephalomyelitis, multiple sclerosis, inflammatory bowel disease, cystic fibrosis, diabetic retinopathy, ischemic-reperfusion injury, vascular restenosis, glomerulonephritis, or gastrointestinal allergy. In some embodiments, the autoimmune disease is adult onset still's disease, systemic juvenile idiopathic arthritis, macrophage activation syndrome, rheumatoid arthritis, Sjogren's syndrome, systemic sclerosis, polymyositis, systemic angitis, mixed connective tissue disease, Crohn's disease, Hashimoto's disease, Grave's disease, Goodpasture's syndrome, Guillain-Barre syndrome, idiopathic thrombocytopenic purpura, irritable bowel syndrome, myasthenia gravis, hypnolepsy, pemphigus vulgaris, pernicious anemia, primary biliary cirrhosis, ulcerative colitis, vasculitis, Wegener's granulomatosis, or psoriasis.

Disclosed herein are methods of treating a cancer in a subject in need thereof, comprising administering an effective amount of the pharmaceutical composition of claim 23 to the subject. In some embodiments, the cancer is multiple myeloma, lung cancer, liver cancer, stomach cancer, colorectal cancer, colon cancer, skin cancer, bladder cancer, prostate cancer, breast cancer, ovarian cancer, cervical cancer, thyroid cancer, kidney cancer, fibrosarcoma, melanoma, or blood cancer.

### BRIEF DESCRIPTION OF DRAWINGS

FIGS. 1A-1B. Heavy chain (FIG. 1A) and light chain (FIG. 1B) expression vectors for the preparation of a recombinant fusion protein.

FIG. 2. A schematic structure of an APB-R3 protein.

FIG. 3. SDS-PAGE results of analyzing the size of the APB-R3 protein in an amount of 1 μg/well and 2 μg/well under reducing (R), non-reducing and boiled (NR(B)), and non-reducing and non-boiled (NR(NB)) conditions.

FIG. 4. SEC-HPLC results of analyzing purity of the APB-R3 protein.

FIG. 5. Results of analyzing an isoelectric point of the APB-R3 protein.

FIG. 6. A graph showing IL-18 inhibition in a KG-1 cell line by the APB-R3 protein.

FIG. 7. A graph showing IL-18 inhibition in mouse CD4+ T cells by the APB-R3 protein.

FIG. 8. A graph showing protein concentrations in blood after subcutaneous administration of the APB-R3 protein into rats.

FIG. 9. A graph showing protein concentrations in blood after intravenous administration of the APB-R3 protein into rats.

FIG. 10. A graph showing body weight of mice in Macrophage activation syndrome (MAS) disease model.

FIGS. 11A-11B. Graphs showing spleen weight/body weight and Liver weight/body weight of mice in Macrophage activation syndrome (MAS) disease model.

FIGS. 12A-12B. Graphs showing the levels of serum Aspartate aminotransferase (AST) and Alanine aminotransferase (ALT) of mice in Macrophage activation syndrome (MAS) disease model.

FIGS. 13A-13B. Graphs showing the levels of serum IFN-γ and CXCL9 of mice in Macrophage activation syndrome (MAS) disease model.

FIG. 14. A graph showing the cell population of splenic monocytes/macrophages of mice in Macrophage activation syndrome (MAS) disease model.

### DETAILED DESCRIPTION

### Antibodies and Fragments Thereof

Disclosed herein are recombinant fusion proteins comprising an interleukin-18-binding protein (IL-18BP) and an antigen binding fragment (Fab) against serum albumin. The fusion proteins can further comprise a linker that links the IL-18BP to the Fab.

In some embodiments, in the fusion proteins disclosed herein, the heavy chain and the light chain of the Fab are bound by a noncovalent bond.

The Fab can comprise
a heavy chain comprising a heavy chain variable domain comprising
(1) a heavy chain complementarity determining domain 1 (CDR1) comprising the amino acid sequence of SYGIS (SEQ ID NO:22),
   a heavy chain complementarity determining domain 2 (CDR2) comprising the amino acid sequence of WINTYSGGTKYAQKFQG (SEQ ID NO:23), and
   a heavy chain complementarity determining domain 3 (CDR3) comprising the amino acid sequence of LGHCQRGICSDALDT (SEQ ID NO:24);
(2) a heavy chain CDR1 comprising the amino acid sequence of SYGIS (SEQ ID NO:22),
   a heavy chain CDR2 comprising the amino acid sequence of RINTYNGNTGYAQRLQG (SEQ ID NO:25), and
   a heavy chain CDR3 comprising the amino acid sequence of LGHCQRGICSDALDT (SEQ ID NO:24);
(3) a heavy chain CDR1 comprising the amino acid sequence of NYGIH (SEQ ID NO:26),
   a heavy chain CDR2 comprising the amino acid sequence of SISYDGSNKYYADSVKG (SEQ ID NO:27), and
   a heavy chain CDR3 comprising the amino acid sequence of DVHYYGSGSYYNAFDI (SEQ ID NO:28);
(4) a heavy chain CDR1 comprising the amino acid sequence of SYAMS (SEQ ID NO:29),
   a heavy chain CDR2 comprising the amino acid sequence of VISHDGGFQYYADSVKG (SEQ ID NO:30), and
   a heavy chain CDR3 comprising the amino acid sequence of AGWLRQYGMDV (SEQ ID NO:31);
(5) a heavy chain CDRlcomprising the amino acid sequence of AYWIA (SEQ ID NO:32),
   a heavy chain CDR2 comprising the amino acid sequence of MIWPPDADARYSPSFQG (SEQ ID NO:33), and
   a heavy chain CDR3 comprising the amino acid sequence of LYSGSYSP (SEQ ID NO:34); or
(6) a heavy chain CDR1 comprising the amino acid sequence of AYSMN (SEQ ID NO:35),
   a heavy chain CDR2 comprising the amino acid sequence of SISSSGRYIHYADSVKG (SEQ ID NO:36), and
   a heavy chain CDR3 comprising the amino acid sequence of ETVMAGKALDY (SEQ ID NO:37); and a light chain comprising a light chain variable domain comprising
(7) a light chain CDR1 comprising the amino acid sequence of RASQSISRYLN (SEQ ID NO:38),
   a light chain CDR2 comprising the amino acid sequence of GASRLES (SEQ ID NO:39), and
   a light chain CDR3 comprising the amino acid sequence of QQSDSVPVT (SEQ ID NO:40);
(8) a light chain CDR1 comprising the amino acid sequence of RASQSISSYLN (SEQ ID NO:41),
   a light chain CDR2 comprising the amino acid sequence of AASSLQS (SEQ ID NO:42), and
   a light chain CDR3 comprising the amino acid sequence of QQSYSTPPYT (SEQ ID NO:43);
(9) a light chain CDR1 comprising the amino acid sequence of RASQSIFNYVA (SEQ ID NO:44),
   a light chain CDR2 comprising the amino acid sequence of DASNRAT (SEQ ID NO:45), and
   a light chain CDR3 comprising the amino acid sequence of QQRSKWPPTWT (SEQ ID NO:46);
(10) a light chain CDR1 comprising the amino acid sequence of RASETVSSRQLA (SEQ ID NO:47),
   a light chain CDR2 comprising the amino acid sequence of GASSRAT (SEQ ID NO:48), and
   a light chain CDR3 comprising the amino acid sequence of QQYGSSPRT (SEQ ID NO:49);
(11) a light chain CDR1 comprising the amino acid sequence ofRASQSVSSSSLA (SEQ ID NO:50),
   a light chain CDR2 comprising the amino acid sequence of GASSRAT (SEQ ID NO:48), and
   a light chain CDR3 comprising the amino acid sequence of QKYSSYPLT (SEQ ID NO:51); or
(12) a light chain CDR1 comprising the amino acid sequence of RASQSVGSNLA (SEQ ID NO:52),
   a light chain CDR2 comprising the amino acid sequence of GASTGAT (SEQ ID NO:53), and
   a light chain CDR3 comprising the amino acid sequence of QQYYSFLAKT (SEQ ID NO:54).

In some embodiments, the heavy chain variable domain comprises a heavy chain CDR1 comprising the amino acid sequence of SEQ ID NO:35, a heavy chain CDR2 comprising the amino acid sequence of SEQ ID NO:36, and a heavy chain CDR3 comprising the amino acid sequence of SEQ ID NO:37, and the light chain variable domain comprises a light chain CDR1 comprising the amino acid sequence of SEQ ID NO:52, a light chain CDR2 comprising the amino acid sequence of SEQ ID NO:53, and a light chain CDR3 comprising the amino acid sequence of SEQ ID NO:54.

In some embodiments, the heavy chain variable domain comprises an amino acid sequence having at least 90% identity to SEQ ID NO:55, 56, 57, 58, 59, or 60. In some embodiments, the light chain variable domain comprises an amino acid sequence having at least 90% identity to SEQ ID NO:61, 62, 63, 64, 65, 66, or 67. In some embodiments, the heavy chain variable domain comprises an amino acid sequence of SEQ ID NO:55, 56, 57, 58, 59, or 60, and the light chain variable domain comprises an amino acid sequence of SEQ ID NO:61, 62, 63, 64, 65, 66, or 67. In some embodiments, the heavy chain constant domain comprises an amino acid sequence having at least 90% identity to SEQ ID NO:68. In some embodiments, the light chain constant domain comprises an amino acid sequence having at least 90% identity to SEQ ID NO:69.

In some embodiments, the heavy chain of the Fab comprises an amino acid sequence of SEQ ID NO:19. In some embodiments, the fusion protein comprises an amino acid sequence of SEQ ID NO:13 and an amino acid sequence of SEQ ID NO:19.

In some embodiments of the recombinant proteins disclosed herein, the heavy chain variable domain comprises a heavy chain CDR1 comprising the amino acid sequence of SEQ ID NO:35, a heavy chain CDR2 comprising the amino acid sequence of SEQ ID NO:36, and a heavy chain CDR3 comprising the amino acid sequence of SEQ ID NO:37, and the light chain variable domain comprises a light chain CDR1 comprising the amino acid sequence of SEQ ID NO:52, a light chain CDR2 comprising the amino acid sequence of SEQ ID NO:53, and a light chain CDR3 comprising the amino acid sequence of SEQ ID NO:54.

In some embodiments, the heavy chain variable domain comprises an amino acid sequence having at least 90%, at least 93%, at least 95%, at least 96%, at least 97%, at least 98%, at least 99%, or 100% identity to SEQ ID NO:55, 56, 57, 58, 59, or 60.

In some embodiments, the light chain variable domain comprises an amino acid sequence having at least 90%, at least 93%, at least 95%, at least 96%, at least 97%, at least 98%, at least 99%, or 100% identity to SEQ ID NO:61, 62, 63, 64, 65, 66, or 67.

In some embodiments, the heavy chain variable domain comprises the amino acid sequence of SEQ ID NO:55, 56, 57, 58, 59, or 60, and the light chain variable domain comprises the amino acid sequence of SEQ ID NO:61, 62, 63, 64, 65, 66 or 67.

In some embodiments, the Fab comprises a heavy chain variable domain comprising an amino acid sequence having at least 90%, at least 93%, at least 95%, at least 96%, at least 97%, at least 98%, at least 99%, or 100% identity to SEQ ID NO:55, 56, 57, 58, 59, or 60, and a light chain variable domain comprising an amino acid sequence having at least 90%, at least 93%, at least 95%, at least 96%, at least 97%, at least 98%, at least 99%, or 100% identity to SEQ ID NO:61, 62, 63, 64, 65, or 66 or 67, respectively, or in any combinations of heavy chain variable domain and light chain variable domain disclosed herein. For example, the Fab can comprise a heavy chain variable domain comprising an amino acid sequence having at least 90%, at least 93%, at least 95%, at least 96%, at least 97%, at least 98%, at least 99%, or 100% identity to SEQ ID NO:60 and a light chain variable domain comprising an amino acid sequence having at least 90%, at least 93%, at least 95%, at least 96%, at least 97%, at least 98%, at least 99%, or 100% identity to SEQ ID NO:67.

In some embodiments, the heavy chain constant domain comprises an amino acid sequence having at least 90%, at least 93%, at least 95%, at least 96%, at least 97%, at least 98%, at least 99%, or 100% identity to SEQ ID NO:68.

In some embodiments, the light chain constant domain comprises an amino acid sequence having at least 90%, at least 93%, at least 95%, at least 96%, at least 97%, at least 98%, at least 99%, or 100% identity to SEQ ID NO:69.

In some embodiments, the recombinant fusion protein can comprise a heavy chain comprising an amino acid sequence having at least 90%, at least 93%, at least 95%, at least 96%, at least 97%, at least 98%, at least 99%, or 100% identity to SEQ ID NO:19. In some embodiments, the Fab comprises a heavy chain domain comprising an amino acid sequence having at least 90%, at least 93%, at least 95%, at least 96%, at least 97%, at least 98%, at least 99%, or 100% identity to SEQ ID NO:10 (V_{H}—C_{H1} domain). In some embodiments, the Fab comprises a light chain domain comprising an amino acid sequence having at least 90%, at least 93%, at least 95%, at least 96%, at least 97%, at least 98%, at least 99%, or 100% identity to SEQ ID NO:13 (V_{L}-C_{κ} domain).

In some embodiments, the recombinant fusion protein can comprise a heavy chain comprising an amino acid sequence having at least 90%, at least 93%, at least 95%, at least 96%, at least 97%, at least 98%, at least 99%, or 100% identity to SEQ ID NO:19; and a light chain comprising an amino acid sequence having at least 90%, at least 93%, at least 95%, at least 96%, at least 97%, at least 98%, at least 99%, or 100% identity to SEQ ID NO:13. The recombinant protein can have significantly improved pharmacokinetic properties while maintaining the intrinsic biological activity of the IL-18BP.

In some embodiments, the Fab comprises a heavy chain domain comprising an amino acid sequence having at least 90%, at least 93%, at least 95%, at least 96%, at least 97%, at least 98%, at least 99%, or 100% identity to SEQ ID NO:10 (V_{H}—C_{H1} domain) and a light chain domain comprising an amino acid sequence having at least 90%, at least 93%, at least 95%, at least 96%, at least 97%, at least 98%, at least 99%, or 100% identity to SEQ ID NO:13 (V_{L}—C_{κ} domain).

As disclosed herein, the recombinant fusion protein comprises an interleukin-18-binding protein and an antigen binding fragment against serum albumin. In some embodiments, the recombinant fusion protein comprises a heavy chain comprising 395 amino acids and a light chain comprising 215 amino acids. In some embodiments, no glycosylation exists in the antigen binding fragment against serum albumin, and 1, 2, 3, or 4 N-glycosylation and one O-glycosylation sites exist in the IL-18-binding protein. Thus, in some embodiments, the recombinant fusion protein can include glycosylation.

As used herein, the term “interleukin-18-binding protein (IL-18BP)” refers to a protein that binds to IL-18 and inhibits binding of IL-18 and IL-18 receptors to exhibit an antagonistic action. In a healthy person, the blood concentration of IL-18-binding protein is known to be as much as 20 times the concentration of IL-18. There are four isoforms of the IL-18-binding protein in humans: a, b, c, and d. Among the four isoforms, type a and c IL-18-binding proteins are known to have high biological activity, i.e., high ability to bind IL-18, and shows a cross-reaction between human IL-18 and murine IL-18. Isoform a has 399 pM of the ability to bind to human IL-18, indicating high levels of binding ability. The IL-18BP can be a non-mutated natural protein or an isoform, which can be obtained from public databases or publications, see, e.g., Kim S.-H. et al., PNAS 97:1190-1195 (2000). In some embodiments, the IL-18BP comprises an amino acid sequence having at least 90%, at least 93%, at least 95%, at least 96%, at least 97%, at least 98%, at least 99%, or 100% identity to SEQ ID NO:7. The IL-18-binding protein can comprise an amino acid sequence having at least 90% identity to SEQ ID NO:7. In some embodiments, the IL-18-binding protein comprises an amino acid sequence of SEQ ID NO:7. In some embodiments, a nucleic acid molecule encoding the IL-18-binding protein comprises a nucleotide sequence of SEQ ID NO:8 or SEQ ID NO:9.

As used herein, the term “linker” refers to a peptide inserted between proteins such that when the recombinant fusion protein is prepared by linking the IL-18-binding protein and the anti-serum albumin Fab antibody fragment, structural flexibility of these proteins can be increased to enhance the activity of each bound protein. There is no limitation on the type of linker or the number of amino acids, as long as it can minimize immune responses. For example, the linker can include 1 amino acid to 20 amino acids, 1 amino acid to 15 amino acids, 1 amino acid to 10 amino acids, or 1 amino acid to 8 amino acids. In some embodiments, the linker can link the IL-18-binding protein at the C-terminus of the heavy chain region of the antigen binding fragment against serum albumin. For example, the linker can include an amino acid sequence of SEQ ID NO:16. A nucleic acid encoding the linker including the amino acid sequence of SEQ ID NO:16 can be represented by SEQ ID NO:17 or SEQ ID NO:18.

In some embodiments, the linker links the IL-18BP to a C-terminus of the heavy chain constant domain, an N-terminus of the heavy chain variable domain, a C-terminus of the light chain constant domain, and/or an N-terminus of the light chain variable domain of the Fab. In some embodiments, the linker links the IL-18BP to a C-terminus of the heavy chain constant domain. In some embodiments, the linker comprises 1 to 50 amino acids. In some embodiments, the linker comprises an amino acid sequence of any one of SEQ ID NOS:16 and 70-84.

Further, the linker can be appropriately modified for use, if needed. For example, the linker can be a polypeptide composed of 1 to 50 or 1 to 20 arbitrary or nonarbitrary amino acids. The peptide linker can include Gly, Asn, and Ser residues, and can also include neutral amino acids such as Thr and Ala. An amino acid sequence suitable for the peptide linker is known in the art. Adjusting the copy number “n” allows for optimization of the linker in order to achieve appropriate separation between the functional moieties or to maintain necessary inter-moiety interaction. Other linkers are known in the art, e.g., G and S linkers containing additional amino acid residues, such as T and A, to maintain flexibility, as well as polar amino acid residues to improve solubility. Therefore, the linker can be a flexible linker containing G, S, and/or T, A residues. The linker can have a general formula of (GpSs)ₙ or (SpGs)ₙ, wherein, independently, p is an integer of 1 to 10, s is 0 or an integer of 0 to 10, p+s is an integer of 20 or less, and n is an integer of 1 to 20. More specifically, examples of the linker can include (GGGGS)ₙ(SEQ ID NO:72), (SGGGG)ₙ(SEQ ID NO:73), (SRSSG)ₙ(SEQ ID NO:74), (SGSSC)ₙ(SEQ ID NO:75), (GKSSGSGSESKS)ₙ(SEQ ID NO:76), (RPPPPC)ₙ(SEQ ID NO:77), (SSPPPPC)ₙ(SEQ ID NO:78), (GSTSGSGKSSEGKG)ₙ(SEQ ID NO:79), (GSTSGSGKSSEGSGSTKG)ₙ(SEQ ID NO:80), (GSTSGSGKPGSGEGSTKG)ₙ(SEQ ID NO:81), or (EGKSSGSGSESKEF)ₙ(SEQ ID NO:82), wherein n can be an integer of 1 to 20, or 1 to 10.

As used herein, the term “serum albumin” is one of proteins constituting basic materials of cells and plays an important role in maintaining the osmotic pressure between blood vessels and tissues by allowing body fluids to stay in blood vessels. In addition, the term “antigen binding fragment against serum albumin” can refer to an anti-serum albumin antibody or an antigen binding fragment of the antibody molecule specifically binding to an epitope of serum albumin.

An antigen binding fragment of an antibody or an antibody fragment refers to a fragment retaining an antigen-binding function, and includes Fab, F(ab′), F(ab′)2, Fv, etc. Fab of the antibody fragments has a structure including variable regions of a light chain and a heavy chain, a constant region of the light chain, and a constant region (CH) of the heavy chain with one antigen-binding site. Fab′ differs from Fab in that it has a hinge region containing one or more cysteine residues at the C-terminal of the heavy chain CH domain. F(ab′)2 antibody is produced when the cysteine residue of the hinge region of Fab′ forms a disulfide bond. Recombinant techniques for generating Fv fragments with minimal antibody fragments having only a heavy chain variable region and a light chain variable region are described in PCT International Publication Nos. WO88/10649, WO88/106630, WO88/07085, WO88/07086, and WO88/09344. In a two-chain Fv, a heavy chain variable region and a light chain variable region are connected via a non-covalent bond. In a single chain Fv (scFv), a heavy chain variable region and a light chain variable region are generally connected via a peptide linker by a covalent bond or directly at the C-terminal. Thus, the single chain Fv (scFv) can have a structure such as a dimer, like the two-chain Fv. Such an antibody fragment can be obtained using a protein hydrolyzing enzyme (for example, when a whole antibody is cleaved with papain, Fab can be obtained, and when a whole antibody is cleaved with pepsin, F(ab′)2 fragment can be obtained), and it can also be produced through a recombinant gene technology.

In some embodiments, the antigen binding fragment against serum albumin can include a heavy chain region comprising an amino acid sequence of SEQ ID NO:10; and a light chain region comprising an amino acid sequence of SEQ ID NO:13. In some embodiments, nucleic acid molecule encoding the heavy chain region comprising the amino acid sequence of SEQ ID NO:10 can have a nucleotide sequence of SEQ ID NO:11 or 12. In some embodiments, nucleic acid molecule encoding the light chain region comprising the amino acid sequence of SEQ ID NO:13 can have a nucleotide sequence of SEQ ID NO:14 or 15.

As used herein, the term “recombinant fusion protein” or “fusion protein” refers to a protein, in which two or more proteins are artificially linked. In some embodiments, the recombinant fusion protein refers to a protein, in which the IL-18-binding protein and the antigen binding fragment against serum albumin, i.e., anti-serum albumin Fab antibody fragment are linked to each other. Such a recombinant fusion protein can be obtained by expressing and purifying the same by chemical synthesis or a genetic recombination method, after each partner is determined. In some embodiments, the recombinant fusion protein can be obtained by expressing, in a cell expression system, a fusion gene (expression vector) in which a gene sequence encoding the IL-18-binding protein and a gene sequence encoding the antigen binding fragment of anti-serum albumin are linked. In the recombinant fusion protein, the IL-18-binding protein and the anti-serum albumin Fab antibody fragment are, either directly or via a linker, linked to each other. In some embodiments, the recombinant fusion protein can include a heavy chain including the IL-18-binding protein, the linker, a heavy chain region of the antigen binding fragment against serum albumin; and a light chain including a light chain region of the antigen binding fragment against serum albumin via a non-covalent bond. For example, the recombinant fusion protein can comprise a peptide including an amino acid sequence of SEQ ID NO:19 and a peptide including an amino acid sequence of SEQ ID NO:13.

As used herein, the terms “antibody” and “antibodies” are terms of art and can be used interchangeably herein and refer to a molecule with an antigen-binding site that specifically binds an antigen. Antibodies can include, e.g., monoclonal antibodies, recombinantly produced antibodies, human antibodies, resurfaced antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, single domain antibodies, monovalent antibodies, single chain antibodies or single-chain Fvs (scFv), camelized antibodies, affybodies, Fab fragments, F(ab′)₂ fragments, disulfide-linked Fvs (sdFv), anti-idiotypic (anti-Id) antibodies (including, e.g., anti-anti-Id antibodies), bispecific antibodies, and multispecific antibodies.

Antibodies can be of any type (e.g., IgG, IgE, IgM, IgD, IgA, or IgY), any class (e.g., IgG₁, IgG₂, IgG₃, IgG₄, IgA₁, or IgA₂), or any subclass (e.g., IgG₂a or IgG₂b) of immunoglobulin molecule.

As used herein, the terms “bioeffector moiety,” “antigen-binding domain,” “antigen-binding region,” “antigen-binding site,” and similar terms refer to the portions of the recombinant protein that comprises the amino acid residues that confer on the recombinant protein its specificity for the antigen (e.g., the complementarity determining regions (CDR)). The antigen-binding region can be derived from any animal species, such as feline, rodents (e.g., mouse, rat, or hamster) and humans.

As used herein, the terms “variable region” or “variable domain” are used interchangeably and are common in the art. The variable region typically refers to a portion of an antibody, generally, a portion of a light or heavy chain, typically about the amino-terminal 110 to 120 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In particular embodiments, the variable region is a primate (e.g., non-human primate) variable region. In certain embodiments, the variable region comprises rodent or murine CDRs and primate (e.g., non-human primate) framework regions (FRs).

The terms “VL” and “VL domain” are used interchangeably to refer to the light chain variable region of an antibody. The terms “VH” and “VH domain” are used interchangeably to refer to the heavy chain variable region of an antibody.

As used herein, the term “heavy chain (HC or CH)” refers to both a full-length heavy chain and a fragment thereof, the full-length heavy chain including a variable region domain VH including an amino acid sequence having a sufficient variable region (VR) sequence to confer specificity for an antigen and three constant region domains CH1, CH2, and CH3. As used herein, the term “light chain (LC or CL)” refers to both a full-length light chain and a fragment thereof, the full-length light chain including a variable region domain VL including an amino acid sequence having a sufficient VR sequence to confer specificity for an antigen and a constant region domain CL.

The heavy chain constant domain and the light chain constant domain can be derived from an IgG1 antibody constant domain, and in any one or more thereof, cysteine which is an amino acid used in a disulfide bond between the light chain and the heavy chain domain can be conserved or deleted or substituted with an amino acid residue other than cysteine. For example, the heavy chain constant domain can comprise an amino acid sequence having at least 90%, at least 93%, at least 95%, at least 96%, at least 97%, at least 98%, at least 99%, or 100% identity to SEQ ID NO:68, and the light chain constant domain can comprise an amino acid sequence having at least 90%, at least 93%, at least 95%, at least 96%, at least 97%, at least 98%, at least 99%, or 100% identity to SEQ ID NO:69. The deletion or substitution of cysteine in the domain can contribute to improving an expression level of the recombinant protein in transformed cells during a process of producing the above-mentioned recombinant protein. In some embodiments, (i) one or more cysteines in the heavy chain constant domain and/or (ii) one or more cysteines in the light chain constant domain that is/are located in an interchain disulfide bond between the light chain and the heavy chain is/are conserved, deleted, and/or substituted with an amino acid residue other than cysteine.

The term “Kabat numbering” and like terms are recognized in the art and refer to a system of numbering amino acid residues in the heavy and light chain variable regions of an antibody, or an antigen-binding portion thereof. In certain aspects, the CDRs of an antibody can be determined according to the Kabat numbering system (see, e.g., Kabat E A & Wu T T (1971) Ann NY Acad Sci 190: 382-391 and Kabat E A et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). In some embodiments, the CDRs of the antibodies described herein have been determined according to the Kabat numbering scheme.

As used herein, the term “constant region” or “constant domain” are interchangeable and have its meaning common in the art. The constant region is an antibody portion, e.g., a carboxyl terminal portion of a light and/or heavy chain, which is not directly involved in binding of an antibody to an antigen but which can exhibit various effector functions, such as interaction with the Fc receptor. The constant region of an immunoglobulin molecule generally has a more conserved amino acid sequence relative to an immunoglobulin variable domain.

As used herein, the term “heavy chain” when used in reference to an antibody can refer to any distinct type, e.g., alpha (a), delta (6), epsilon (F), gamma (γ), and mu (p), based on the amino acid sequence of the constant domain, which give rise to IgA, IgD, IgE, IgG, and IgM classes of antibodies, respectively, including subclasses of IgG, e.g., IgG₁, IgG₂, IgG₃, and IgG₄.

As used herein, the term “light chain” when used in reference to an antibody can refer to any distinct type, e.g., kappa (C_{κ}) or lambda (Cλ) based on the amino acid sequence of the constant domains. Light chain amino acid sequences are well known in the art. In specific embodiments, the light chain is a human light chain.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured and/or expressed in a number of ways known in the art, including, but not limited to, equilibrium dissociation constant (K_{D}), and equilibrium association constant (KA). The K_{D} is calculated from the quotient of k_{off}/kₒₙ, whereas KA is calculated from the quotient of kₒₙ/k_{off}. kₒₙ refers to the association rate constant of, e.g., an antibody to an antigen, and k_{off} refers to the dissociation of, e.g., an antibody to an antigen. The kₒₙ and k_{off} can be determined by techniques known to one of ordinary skill in the art, such as BIAcore© or KinExA.

In some embodiments, the binding affinity of the recombinant fusion proteins disclosed herein has a binding affinity that is at least 1.5-fold, at least 2-fold, at least 3-fold, at least 4-fold, at least 5-fold, at least 6-fold, at least 7-fold, at least 8-fold, at least 9-fold, at least 10-fold, or any ranges therein higher than that of human IL-18BPa, e.g., 2-fold to 10-fold higher.

As used herein, a “conservative amino acid substitution” is one in which the amino acid residue is replaced with an amino acid residue having a similar side chain. Families of amino acid residues having side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). In certain embodiments, one or more amino acid residues within a CDR(s) or within a framework region(s) of an antibody can be replaced with an amino acid residue with a similar side chain.

As used herein, an “epitope” is a term in the art and refers to a localized region of an antigen to which an antibody can specifically bind. An epitope can be, e.g., contiguous amino acids of a polypeptide (linear or contiguous epitope) or an epitope can, e.g., come together from two or more non-contiguous regions of a polypeptide or polypeptides (conformational, non-linear, discontinuous, or non-contiguous epitope). In certain embodiments, the epitope to which an antibody binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligopeptide scanning assays, and/or mutagenesis mapping (e.g., site-directed mutagenesis mapping). For X-ray crystallography, crystallization can be accomplished using any of the known methods in the art (e.g., Giegé R et al., (1994) Acta Crystallogr D Biol Crystallogr 50(Pt 4):339-350; McPherson A (1990) Eur J Biochem 189:1-23; Chayen N E (1997) Structure 5:1269-1274; McPherson, A. (1976) J. Biol. Chem. 251:6300-6303). Antibody:antigen crystals can be studied using well known X-ray diffraction techniques and can be refined using computer software such as X-PLOR (Yale University, 1992, distributed by Molecular Simulations, Inc.; see, e.g., Meth Enzymol (1985) volumes 114 & 115, eds Wyckoff H W et al.; U.S. 2004/0014194), and BUSTER (Bricogne G (1993) Acta Crystallogr D Biol Crystallogr 49 (Pt 1):37-60; Bricogne G (1997) Meth Enzymol 276A:361-423, ed Carter C W; Roversi P et al., (2000) Acta Crystallogr D Biol Crystallogr 56 (Pt 10):1316-1323). Mutagenesis mapping studies can be accomplished using any method known to one of skill in the art. See, e.g., Champe M et al., (1995) J Biol Chem 270:1388-1394 and Cunningham B C & Wells J A (1989) Science 244:1081-1085 for a description of mutagenesis techniques, including alanine scanning mutagenesis techniques. In some embodiments, the epitope of an antibody is determined using alanine scanning mutagenesis studies.

As used herein, the terms “immunospecifically binds,” “immunospecifically recognizes,” “specifically binds,” and “specifically recognizes” are analogous terms in the context of antibodies and refer to molecules that bind to an antigen (e.g., epitope, immune complex, or binding partner of an antigen-binding site) as such binding is understood by one skilled in the art. For example, a molecule that specifically binds to an antigen can bind to other peptides or polypeptides, generally with lower affinity as determined by, e.g., immunoassays, BIAcore®, KinExA 3000 instrument (Sapidyne Instruments, Boise, ID), or other assays known in the art. In some embodiments, molecules that immunospecifically bind to an antigen bind to the antigen with a K_{A} that is at least 2 logs, 2.5 logs, 3 logs, 4 logs or greater than the K_{A} when the molecules bind to another antigen.

In some embodiments, molecules that immunospecifically bind to an antigen do not cross react with other proteins under similar binding conditions. In some embodiments, molecules that immunospecifically bind to an antigen do not cross react with other proteins. In some embodiments, provided herein are recombinant proteins that bind to a specified antigen with higher affinity than to another unrelated antigen. In certain embodiments, provided herein is a recombinant protein that binds to a specified antigen (e.g., human serum albumin) with a 20%, 25%, 30%, 35%, 40%, 45%, 50%, 55%, 60%, 65%, 70%, 75%, 80%, 85%, 90%, 95% or higher affinity than to another, unrelated antigen as measured by, e.g., a radioimmunoassay, surface plasmon resonance, or kinetic exclusion assay. In some embodiments, the extent of binding of a recombinant protein described herein to an unrelated, protein is less than 10%, 15%, or 20% of the binding of the antibody to the specified antigen as measured by, e.g., a radioimmunoassay.

In some embodiments, provided herein are recombinant proteins that bind to an antigen of various species, such as feline, rodents (e.g., mouse, rat, or hamster) and humans. In some embodiments, provided herein are recombinant proteins that bind to a human antigen with higher affinity than to another species of the antigen. In certain embodiments, provided herein are recombinant proteins that bind to a human antigen with a 5%, 10%, 15%, 20%, 25%, 30%, 35%, 40%, 45%, 50%, 55%, 60%, 65%, 70% or higher affinity than to another species as measured by, e.g., a radioimmunoassay, surface plasmon resonance, or kinetic exclusion assay. In some embodiments, the recombinant proteins described herein, which bind to a human antigen, will bind to another species of the antigen protein with less than 10%, 15%, or 20% of the binding of the antibody to the human antigen protein as measured by, e.g., a radioimmunoassay, surface plasmon resonance, or kinetic exclusion assay.

As used herein, the term “host cell” can be any type of cell, e.g., a primary cell, a cell in culture, or a cell from a cell line. In embodiments, the term “host cell” refers to a cell transfected with a nucleic acid molecule and the progeny or potential progeny of such a cell. Progeny of such a cell cannot be identical to the parent cell transfected with the nucleic acid molecule, e.g., due to mutations or environmental influences that can occur in succeeding generations or integration of the nucleic acid molecule into the host cell genome.

In certain aspects, a recombinant protein described herein can be described by its VL domain alone, or its VH domain alone, or by its 3 VL CDRs alone, or its 3 VH CDRs alone. See, e.g., Rader C et al., (1998) PNAS 95: 8910-8915, which is incorporated herein by reference in its entirety, describing the humanization of the mouse anti-avP3 antibody by identifying a complementing light chain or heavy chain, respectively, from a human light chain or heavy chain library, resulting in humanized antibody variants having affinities as high or higher than the affinity of the original antibody. See also Clackson T et al., (1991) Nature 352:624-628, which is incorporated herein by reference in its entirety, describing methods of producing antibodies that bind a specific antigen by using a specific VL domain (or VH domain) and screening a library for the complementary variable domains. The screen produced 14 new partners for a specific VH domain and 13 new partners for a specific VL domain, which were strong binders, as determined by ELISA. See also Kim S J & Hong H J, (2007) J Microbiol 45:572-577, which is incorporated herein by reference in its entirety, describing methods of producing antibodies that bind a specific antigen by using a specific VH domain and screening a library (e.g., human VL library) for complementary VL domains; the selected VL domains in turn could be used to guide selection of additional complementary (e.g., human) VH domains.

In certain aspects, the CDRs of an antibody can be determined according to the Chothia numbering scheme, which refers to the location of immunoglobulin structural loops (see, e.g., Chothia C & Lesk A M, (1987), J Mol Biol 196: 901-917; Al-Lazikani B et al., (1997) J Mol Biol 273: 927-948; Chothia C et al., (1992) J Mol Biol 227: 799-817; Tramontano A et al., (1990) J Mol Biol 215(1): 175-82; and U.S. Pat. No. 7,709,226). Typically, when using the Kabat numbering convention, the Chothia CDR-H1 loop is present at heavy chain amino acids 26 to 32, 33, or 34, the Chothia CDR-H2 loop is present at heavy chain amino acids 52 to 56, and the Chothia CDR-H3 loop is present at heavy chain amino acids 95 to 102, while the Chothia CDR-L1 loop is present at light chain amino acids 24 to 34, the Chothia CDR-L2 loop is present at light chain amino acids 50 to 56, and the Chothia CDR-L3 loop is present at light chain amino acids 89 to 97. The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34).

In certain aspects, provided herein are recombinant proteins that specifically bind to serum albumin (e.g., human serum albumin) and comprise the Chothia VL CDRs of a VL. In certain aspects, provided herein are antibodies that specifically bind to serum albumin (e.g., human serum albumin) and comprise the Chothia VH CDRs of a VH. In certain aspects, provided herein are antibodies that specifically bind to serum albumin (e.g., human serum albumin) and comprise the Chothia VL CDRs of a VL and comprise the Chothia VH CDRs of a VH. In certain embodiments, antibodies that specifically bind to serum albumin (e.g., human serum albumin) comprise one or more CDRs, in which the Chothia and Kabat CDRs have the same amino acid sequence. In certain embodiments, provided herein are antibodies that specifically bind to serum albumin and comprise combinations of Kabat CDRs and Chothia CDRs.

In certain aspects, the CDRs of an antibody can be determined according to the IMGT numbering system as described in Lefranc M-P, (1999) The Immunologist 7: 132-136 and Lefranc M-P et al., (1999) Nucleic Acids Res 27: 209-212. According to the IMGT numbering scheme, VH-CDR1 is at positions 26 to 35, VH-CDR2 is at positions 51 to 57, VH-CDR3 is at positions 93 to 102, VL-CDR1 is at positions 27 to 32, VL-CDR2 is at positions 50 to 52, and VL-CDR3 is at positions 89 to 97.

In certain aspects, the CDRs of an antibody can be determined according to MacCallum R M et al., (1996) J Mol Biol 262: 732-745. See also, e.g., Martin A. “Protein Sequence and Structure Analysis of Antibody Variable Domains,” in Antibody Engineering, Kontermann and Dübel, eds., Chapter 31, pp. 422-439, Springer-Verlag, Berlin (2001).

In certain aspects, the CDRs of an antibody can be determined according to the AbM numbering scheme, which refers AbM hypervariable regions that represent a compromise between the Kabat CDRs and Chothia structural loops and are used by Oxford Molecular's AbM antibody modeling software (Oxford Molecular Group, Inc.).

In some embodiments, the position of one or more CDRs along the VH (e.g., CDR1, CDR2, or CDR3) and/or VL (e.g., CDR1, CDR2, or CDR3) region of an antibody described herein can vary by one, two, three, four, five, or six amino acid positions so long as immunospecific binding to an antigen is maintained (e.g., substantially maintained, e.g., at least 50%, at least 60%, at least 70%, at least 80%, at least 90%, at least 95%). For example, the position defining a CDR of an antibody described herein can vary by shifting the N-terminal and/or C-terminal boundary of the CDR by one, two, three, four, five, or six amino acids, relative to the CDR position of an antibody described herein, so long as immunospecific binding to the antigen(s) is maintained (e.g., substantially maintained, e.g., at least 50%, at least 60%, at least 70%, at least 80%, at least 90%, at least 95%). In other embodiments, the length of one or more CDRs along the VH (e.g., CDR1, CDR2, or CDR3) and/or VL (e.g., CDR1, CDR2, or CDR3) region of an antibody described herein can vary (e.g., be shorter or longer) by one, two, three, four, five, or more amino acids, so long as immunospecific binding to the antigen(s) is maintained (e.g., substantially maintained, e.g., at least 50%, at least 60%, at least 70%, at least 80%, at least 90%, at least 95%).

In some embodiments, a VL CDR1, VL CDR2, VL CDR3, VH CDR1, VH CDR2, and/or VH CDR3 described herein can be one, two, three, four, five or more amino acids shorter than one or more of the CDRs described herein so long as immunospecific binding to the antigen(s) is maintained (e.g., substantially maintained, e.g., at least 50%, at least 60%, at least 70%, at least 80%, at least 90%, at least 95%). In other embodiments, a VL CDR1, VL CDR2, VL CDR3, VH CDR1, VH CDR2, and/or VH CDR3 described herein can be one, two, three, four, five or more amino acids longer than one or more of the CDRs described herein so long as immunospecific binding to the antigen(s) is maintained (e.g., substantially maintained, e.g., at least 50%, at least 60%, at least 70%, at least 80%, at least 90%, at least 95%). In other embodiments, the amino terminus of a VL CDR1, VL CDR2, VL CDR3, VH CDR1, VH CDR2, and/or VH CDR3 described herein can be extended by one, two, three, four, five or more amino acids compared to one or more of the CDRs described herein so long as immunospecific binding to the antigen(s) is maintained (e.g., substantially maintained, e.g., at least 50%, at least 60%, at least 70%, at least 80%, at least 90%, at least 95%). In other embodiments, the carboxy terminus of a VL CDR1, VL CDR2, VL CDR3, VH CDR1, VH CDR2, and/or VH CDR3 described herein can be extended by one, two, three, four, five or more amino acids compared to one or more of the CDRs described herein so long as immunospecific binding to the antigen(s) is maintained (e.g., substantially maintained, e.g., at least 50%, at least 60%, at least 70%, at least 80%, at least 90%, at least 95%). In other embodiments, the amino terminus of a VL CDR1, VL CDR2, VL CDR3, VH CDR1, VH CDR2, and/or VH CDR3 described herein can be shortened by one, two, three, four, five or more amino acids compared to one or more of the CDRs described herein so long as immunospecific binding to the antigen(s) is maintained (e.g., substantially maintained, e.g., at least 50%, at least 60%, at least 70%, at least 80%, at least 90%, at least 95%). In some embodiments, the carboxy terminus of a VL CDR1, VL CDR2, VL CDR3, VH CDR1, VH CDR2, and/or VH CDR3 described herein can be shortened by one, two, three, four, five or more amino acids compared to one or more of the CDRs described herein so long as immunospecific binding to the antigen(s) is maintained (e.g., substantially maintained, e.g., at least 50%, at least 60%, at least 70%, at least 80%, at least 90%, at least 95%). Any method known in the art can be used to ascertain whether immunospecific binding to the antigen(s) is maintained, e.g., the binding assays and conditions described in the “Examples” section herein.

The determination of percent identity between two sequences (e.g., amino acid sequences or nucleic acid sequences) can also be accomplished using a mathematical algorithm. A specific, non-limiting example of a mathematical algorithm utilized for the comparison of two sequences is the algorithm of Karlin S & Altschul S F (1990) PNAS 87: 2264-2268, modified as in Karlin S & Altschul S F (1993) PNAS 90: 5873-5877. Such an algorithm is incorporated into the NBLAST and XBLAST programs of Altschul S F et al., (1990) J Mol Biol 215: 403. BLAST nucleotide searches can be performed with the NBLAST nucleotide program parameters set, e.g., for score=100, wordlength=12 to obtain nucleotide sequences homologous to nucleic acid molecules described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul S F et al., (1997) Nuc Acids Res 25: 3389 3402. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another specific, nonlimiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

The recombinant proteins disclosed herein can be fused or conjugated (e.g., covalently or noncovalently linked) to a detectable label or substance. Examples of detectable labels or substances include enzyme labels, such as, glucose oxidase; radioisotopes, such as iodine (¹²⁵I, ¹²¹I), carbon (¹⁴C), sulfur (³⁵S), tritium (³H), indium (¹²¹In), and technetium (⁹⁹Tc); luminescent labels, such as luminol; and fluorescent labels, such as fluorescein and rhodamine, and biotin. Such labeled antibodies can be used to detect antigen proteins.

### Antibody Production

According to one exemplary embodiment, a recombinant protein (APB-R3) was prepared, the recombinant protein (APB-R3) including an antigen binding fragment binding to human serum albumin, wherein the antigen binding fragment is bound with a heavy chain constant domain and a light chain constant domain; and an IL-18BP linked to the heavy chain constant domain. It was confirmed that the recombinant protein was obtained in a high yield while maintaining biological activities possessed by the respective factors.

Still other aspects provide methods of preparing the recombinant protein, the methods including (a) culturing the cells; and (b) recovering the recombinant protein from the cultured cells. The cells can be cultured in various media. A commercially available medium can be used as a culture medium without limitation. All other essential supplements known to those skilled in the art can also be included at appropriate concentrations. Culture conditions, e.g., temperature, pH, etc., are those previously used together with the host cell selected for expression, and will be apparent to those skilled in the art. The recovering of the recombinant proteins can be performed by removing impurities by, e.g., centrifugation or ultrafiltration, and purifying the resultant by, e.g., affinity chromatography, etc. Other additional purification techniques, e.g., anion or cation exchange chromatography, hydrophobic interaction chromatography, hydroxylapatite chromatography, etc. can be used.

Recombinant proteins disclosed herein can be produced by any method known in the art for the synthesis of antibodies, e.g., by chemical synthesis or by recombinant expression techniques. The methods described herein employ, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described, e.g., in the references cited herein and are fully explained in the literature. See, e.g., Maniatis T et al., (1982) Molecular Cloning: A Laboratory Manual, Cold Spring Harbor Laboratory Press; Sambrook J et al., (1989), Molecular Cloning: A Laboratory Manual, Second Edition, Cold Spring Harbor Laboratory Press; Sambrook J et al., (2001) Molecular Cloning: A Laboratory Manual, Cold Spring Harbor Laboratory Press, Cold Spring Harbor, NY; Ausubel F M et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, TRL Press; Eckstein (ed.) (1991) Oligonucleotides and Analogues: A Practical Approach, TRL Press; Birren B et al., (eds.) (1999) Genome Analysis: A Laboratory Manual, Cold Spring Harbor Laboratory Press.

In some embodiments, the recombinant proteins described herein are antibodies (e.g., recombinant antibodies) prepared, expressed, created, or isolated by any means that involves creation, e.g., via synthesis or genetic engineering of DNA sequences. In certain embodiments, such antibodies comprise sequences (e.g., DNA sequences or amino acid sequences) that do not naturally exist within the antibody germline repertoire of an animal or mammal (e.g., human) in vivo.

In some aspects, provided herein are methods of making recombinant proteins disclosed herein comprising culturing a cell or host cell as described herein. In some aspects, provided herein are methods of making a recombinant protein comprising expressing (e.g., recombinantly expressing) the antibodies using a cell or host cell described herein (e.g., a cell or a host cell comprising polynucleotides encoding an antibody described herein). In some embodiments, the cell is an isolated cell. In some embodiments, the exogenous polynucleotides have been introduced into the cell. In some embodiments, the method further comprises purifying the antibody obtained from the cell or host cell.

Antibodies can be prepared using a wide variety of techniques known in the art including the use of hybridoma, recombinant, and phage display technologies, or a combination thereof. For example, monoclonal antibodies can be produced using hybridoma techniques including those known in the art and taught, e.g., in Harlow E & Lane D, Antibodies: A Laboratory Manual (Cold Spring Harbor Laboratory Press, 2nd ed. 1988); Hammerling G J et al., in: Monoclonal Antibodies and T-Cell Hybridomas 563 681 (Elsevier, N.Y., 1981). The term “monoclonal antibody” as used herein is not limited to antibodies produced through hybridoma technology. For example, monoclonal antibodies can be produced recombinantly from host cells exogenously expressing an antibody described herein.

A “monoclonal antibody,” as used herein, is an antibody produced by a single cell (e.g., hybridoma or host cell producing a recombinant antibody), wherein the antibody immunospecifically binds to an antigen (e.g., human serum albumin) as determined, e.g., by ELISA or other antigen-binding or competitive binding assay known in the art or in the Examples provided herein. In particular embodiments, a monoclonal antibody can be a chimeric antibody or a humanized antibody. In certain embodiments, a monoclonal antibody is a monovalent antibody or multivalent (e.g., bivalent) antibody. In certain embodiments, a monoclonal antibody can be a Fab fragment or a F(ab′)₂ fragment. Monoclonal antibodies described herein can, e.g., be made by the hybridoma method as described in Kohler G & Milstein C (1975) Nature 256: 495 or can, e.g., be isolated from phage libraries using the techniques as described herein, for example. Other methods for the preparation of clonal cell lines and of monoclonal antibodies expressed thereby are well known in the art (see, e.g., Chapter 11 in: Short Protocols in Molecular Biology, (2002) 5th Ed., Ausubel F M et al., supra).

Methods for producing and screening for specific antibodies using hybridoma technology are routine and well known in the art. For example, in the hybridoma method, a mouse or other appropriate host animal, such as a sheep, goat, rabbit, rat, hamster or macaque monkey, is immunized to elicit lymphocytes that produce or are capable of producing antibodies that will specifically bind to the antigen (e.g., human serum albumin)) used for immunization. Alternatively, lymphocytes can be immunized in vitro. Lymphocytes then are fused with myeloma cells using a suitable fusing agent, such as polyethylene glycol, to form a hybridoma cell (Goding J W (Ed), Monoclonal Antibodies: Principles and Practice, pp. 59-103 (Academic Press, 1986)). Additionally, a RIMMS (repetitive immunization multiple sites) technique can be used to immunize an animal (Kilpatrick K E et al., (1997) Hybridoma 16:381-9, incorporated by reference in its entirety).

Antibodies described herein can be generated by any technique known to those of skill in the art. For example, Fab and F(ab′)₂ fragments described herein can be produced by proteolytic cleavage of immunoglobulin molecules, using enzymes such as papain (to produce Fab fragments) or pepsin (to produce F(ab′)₂ fragments). A Fab fragment corresponds to one of the two identical arms of a tetrameric antibody molecule and contains the complete light chain paired with the VH and CH1 domains of the heavy chain. A F(ab′)₂ fragment contains the two antigen-binding arms of a tetrameric antibody molecule linked by disulfide bonds in the hinge region.

Further, the antibodies described herein can also be generated using various phage display methods known in the art. In phage display methods, proteins are displayed on the surface of phage particles which carry the polynucleotide sequences encoding them. In particular, DNA sequences encoding VH and VL domains are amplified from cDNA libraries (e.g., human or murine cDNA libraries of affected tissues). The DNA encoding the VH and VL domains are recombined together with a scFv linker by PCR and cloned into a phagemid vector. The vector is electroporated in E. coli and the E. coli is infected with helper phage. Phage used in these methods are typically filamentous phage including fd and M13, and the VH and VL domains are usually recombinantly fused to either the phage gene III or gene VIII. Phage expressing an antibody that binds to a particular antigen can be selected or identified with antigen, e.g., using labeled antigen or antigen bound or captured to a solid surface or bead. Examples of phage display methods that can be used to make the antibodies described herein include those disclosed in Brinkman U et al., (1995) J Immunol Methods 182: 41-50; Ames R S et al., (1995) J Immunol Methods 184: 177-186; Kettleborough C A et al., (1994) Eur J Immunol 24: 952-958; Persic L et al., (1997) Gene 187: 9-18; Burton D R & Barbas C F (1994) Advan Immunol 57: 191-280; PCT/GB91/001134; WO90/02809, WO91/10737, WO92/01047, WO92/18619, WO93/11236, WO95/15982, WO95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743, and 5,969,108.

As described in the above references, after phage selection, the antibody coding regions from the phage can be isolated and used to generate antibodies, including human antibodies, and expressed in any desired host, including mammalian cells, insect cells, plant cells, yeast, and bacteria, e.g., as described below. Techniques to recombinantly produce antibodies such as Fab, Fab′ and F(ab′)₂ fragments can also be employed using methods known in the art such as those disclosed in WO92/22324; Mullinax R L et al., (1992) BioTechniques 12(6): 864-9; Sawai H et al., (1995) Am J Reprod Immunol 34: 26-34; and Better M et al., (1988) Science 240: 1041-1043.

In some aspects, to generate antibodies, PCR primers including VH or VL nucleotide sequences, a restriction site, and a flanking sequence to protect the restriction site can be used to amplify the VH or VL sequences from a template, e.g., scFv clones. Utilizing cloning techniques known to those of skill in the art, the PCR amplified VH domains can be cloned into vectors expressing a VH constant region, and the PCR amplified VL domains can be cloned into vectors expressing a VL constant region, e.g., human kappa or lambda constant regions. The VH and VL domains can also be cloned into one vector expressing the necessary constant regions. The heavy chain conversion vectors and light chain conversion vectors are then co-transfected into cell lines to generate stable or transient cell lines that express antibodies, e.g., IgG, using techniques known to those of skill in the art.

A chimeric antibody is a molecule in which different portions of the antibody are derived from different immunoglobulin molecules. For example, a chimeric antibody can contain a variable region of a human monoclonal antibody fused to a constant region of a human antibody. Methods for producing chimeric antibodies are known in the art. See, e.g., Morrison S L (1985) Science 229: 1202-7; Oi V T & Morrison S L (1986) BioTechniques 4: 214-221; Gillies S D et al., (1989) J Immunol Methods 125: 191-202; and U.S. Pat. Nos. 5,807,715, 4,816,567, 4,816,397, and 6,331,415.

### Polynucleotides, Vectors, and Cells

Disclosed herein are nucleic acid molecules encoding the recombinant proteins disclosed herein.

Disclosed herein are expression vectors comprising the nucleic acid molecules disclosed herein.

Disclosed herein are cells transformed with the expression vectors disclosed herein.

Since the nucleic acid, the expression vector, and the transformed cell include the above-described recombinant protein or the nucleic acid encoding the recombinant protein as it is, or they use the same, descriptions common thereto will be omitted.

For example, in some aspects, the recombinant protein can be produced by isolating the nucleic acid encoding the recombinant protein. The nucleic acid is isolated and inserted into a replicable vector to perform additional cloning (DNA amplification) or additional expression. On the basis of this, other aspects relate to a vector including the nucleic acid.

As used herein, the term “nucleic acid” or “nucleic acid molecule” comprehensively includes DNA (gDNA and cDNA) and RNA molecules, and nucleotides as basic units of the nucleic acid include not only natural nucleotides but also analogues having modified sugar or base moieties.

The nucleic acid is interpreted to include a nucleotide sequence showing substantial identity to the nucleotide sequence. Substantial identity means a nucleotide sequence showing at least 80% homology, more specifically at least 90% homology, and most specifically at least 95% homology, when the nucleotide sequence of the present disclosure and another optional sequence are aligned to correspond to each other as much as possible and the aligned sequences are analyzed using an algorithm commonly used in the art.

DNA encoding the recombinant protein is easily isolated or synthesized by using a common process (e.g., by using an oligonucleotide probe capable of specifically binding to the DNA encoding the recombinant protein). Many vectors are available. Vector components generally include, but are not limited to, one or more of the following: a signal sequence, an origin of replication, one or more marker genes, an enhancer element, a promoter, and a transcription termination sequence.

As used herein, the term “vector” includes, as a means to express a target gene in a host cell, plasmid vectors; cosmid vectors; viral vectors such as bacteriophage vectors, adenovirus vectors, retrovirus vectors, and adeno-associated virus vectors, etc. In the vector, the nucleic acid encoding the recombinant protein is operably linked to a promoter.

“Operably linked” refers to a functional linkage between a nucleic acid expression control sequence (e.g., a promoter, a signal sequence, an array of transcriptional regulatory factor binding sites) and another nucleic acid sequence, whereby the control sequence directs transcription and/or translation of another nucleic acid sequence.

When a prokaryotic cell is used as a host, a powerful promoter capable of directing transcription (e.g., tac promoter, lac promoter, lacUV5 promoter, lpp promoter, pLλ promoter, pRλ promoter, rac5 promoter, amp promoter, recA promoter, SP6 promoter, trp promoter and T7 promoter, etc.), a ribosome binding site for initiation of translation, and a transcription/translation termination sequence are generally included. For example, when a eukaryotic cell is used as a host, a promoter derived from the genome of a mammalian cell (e.g., metallothionein promoter, β-actin promoter, human hemoglobin promoter, and human muscle creatine promoter) or a promoter derived from mammalian viruses (e.g., adenovirus late promoter, vaccinia virus 7.5K promoter, SV40 promoter, cytomegalovirus (CMV) promoter, tk promoter of HSV, mouse mammary tumor virus (MMTV) promoter, LTR promoter of HIV, promoter of Moloney virus, promoter of Epstein-Barr virus (EBV), and promoter of Rous sarcoma virus (RSV)) can be used, and a polyadenylated sequence can be commonly used as the transcription termination sequence. In some cases, the vector can be fused with another sequence to facilitate purification of the recombinant protein expressed therefrom. The sequence to be fused includes, e.g., glutathione S-transferase (Pharmacia, USA), maltose binding protein (NEB, USA), FLAG (IBI, USA), 6×His (hexahistidine; Quiagen, USA), etc. The vector includes, as a selective marker, an antibiotic-resistant gene that is ordinarily used in the art, e.g., genes resistant against ampicillin, gentamycin, carbenicillin, chloramphenicol, streptomycin, kanamycin, geneticin, neomycin, and tetracycline.

In still other aspects, the present disclosure provides cells transformed with the above-mentioned vectors. The cells used to produce the recombinant protein of the present disclosure can be prokaryotic cells, yeast cells, or higher eukaryotic cells, but are not limited thereto. Prokaryotic host cells such as Escherichia coli, the genus bacillus strains such as Bacillus subtilis and Bacillus thuringiensis, Streptomyces, Pseudomonas (e.g., Pseudomonas putida), Proteus mirabilis and Staphylococcus (e.g., Staphylococcus carnosus) can be used. However, animal cells are most interested, and examples of the useful host cell line can include COS-7, BHK, CHO (GS null CHO-K1), CHOK1, DXB-11, DG-44, CHO/-DHFR, CV1, COS-7, HEK293, BHK, TM4, VERO, HELA, MDCK, BRL 3A, W138, Hep G2, SK-Hep, MMT, TRI, MRC 5, FS4, 3T3, RIN, A549, PC12, K562, PER.C6, SP2/0, NS-0, U20S, or HT1080, but are not limited thereto.

As used herein, the term “transformation” means a molecular biological technique that changes the genetic trait of a cell by a DNA chain fragment or plasmid which possesses a different type of foreign gene from that of the original cell, penetrates among the cells, and combines with DNA in the original cell. The transformation means insertion of the expression vector including the gene of the recombinant protein into a host cell.

Provided herein are nucleic acid molecules comprising a nucleotide sequence encoding a recombinant protein described herein (e.g., a variable light chain region and/or variable heavy chain region) that immunospecifically binds to an antigen, and vectors, e.g., vectors comprising such polynucleotides for recombinant expression in host cells (e.g., E. coli and mammalian cells). Provided herein are polynucleotides comprising nucleotide sequences encoding any of the antibodies provided herein, as well as vectors comprising such polynucleotide sequences, e.g., expression vectors for their efficient expression in host cells, e.g., mammalian cells.

As used herein, an “isolated” polynucleotide or nucleic acid molecule is one which is separated from other nucleic acid molecules which are present in the natural source (e.g., in a mouse or a human) of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. For example, the language “substantially free” includes preparations of polynucleotide or nucleic acid molecule having less than about 15%, 10%, 5%, 2%, 1%, 0.5%, or 0.1% (in particular less than about 10%) of other material, e.g., cellular material, culture medium, other nucleic acid molecules, chemical precursors and/or other chemicals. In some embodiments, a nucleic acid molecule(s) encoding an antibody described herein is isolated or purified.

Provided herein are polynucleotides comprising nucleotide sequences encoding antibodies, which immunospecifically bind to an antigen polypeptide (e.g., human serum albumin) and comprises an amino acid sequence as described herein, as well as antibodies that compete with such antibodies for binding to an antigen polypeptide (e.g., in a dose-dependent manner), or which binds to the same epitope as that of such antibodies.

Provided herein are polynucleotides comprising a nucleotide sequence encoding the light chain or heavy chain of an antibody described herein. The polynucleotides can comprise nucleotide sequences encoding a light chain comprising the VL FRs and CDRs of antibodies described herein. The polynucleotides can comprise nucleotide sequences encoding a heavy chain comprising the VH FRs and CDRs of antibodies described herein.

Provided herein are polynucleotides comprising a nucleotide sequence encoding a recombinant protein comprising a Fab comprising three VH chain CDRs, e.g., containing VL CDR1, VL CDR2, and VL CDR3 of an antibody to human serum albumin described herein and three VH chain CDRs, e.g., containing VH CDR1, VH CDR2, and VH CDR3 of an antibody to human serum albumin described herein.

Provided herein are polynucleotides comprising a nucleotide sequence encoding a recombinant protein comprising a VL domain.

In certain embodiments, a polynucleotide described herein comprises a nucleotide sequence encoding a recombinant protein provided herein comprising a light chain variable region comprising an amino acid sequence described herein (e.g., SEQ ID NO:61, 62, 63, 64, 65, 66, or 67), wherein the antibody immunospecifically binds to serum albumin.

In certain embodiments, a polynucleotide described herein comprises a nucleotide sequence encoding an antibody provided herein comprising a heavy chain variable region comprising an amino acid sequence described herein (e.g., SEQ ID NO:55, 56, 57, 58, 59, or 60), wherein the antibody immunospecifically binds to serum albumin.

In specific aspects, provided herein are polynucleotides comprising a nucleotide sequence encoding an antibody comprising a light chain and a heavy chain, e.g., a separate light chain and heavy chain. With respect to the light chain, in some embodiments, a polynucleotide provided herein comprises a nucleotide sequence encoding a kappa light chain.

In other embodiments, a polynucleotide provided herein comprises a nucleotide sequence encoding a lambda light chain. In yet other embodiments, a polynucleotide provided herein comprises a nucleotide sequence encoding an antibody described herein comprising a human kappa light chain or a human lambda light chain. In some embodiments, a polynucleotide provided herein comprises a nucleotide sequence encoding an antibody, which immunospecifically binds to serum albumin, wherein the antibody comprises a light chain, and wherein the amino acid sequence of the VL domain can comprise the amino acid sequence set forth in SEQ ID NO:61, 62, 63, 64, 65, 66, or 67 and wherein the constant region of the light chain comprises the amino acid sequence of a kappa light chain constant region.

Also provided herein are polynucleotides encoding an antibody or a fragment thereof that are optimized, e.g., by codon/RNA optimization, replacement with heterologous signal sequences, and elimination of mRNA instability elements. Methods to generate optimized nucleic acids encoding an antibody or a fragment thereof (e.g., light chain, heavy chain, VH domain, or VL domain) for recombinant expression by introducing codon changes and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly. For example, potential splice sites and instability elements (e.g., A/T or A/U rich elements) within the RNA can be mutated without altering the amino acids encoded by the nucleic acid sequences to increase stability of the RNA for recombinant expression. The alterations utilize the degeneracy of the genetic code, e.g., using an alternative codon for an identical amino acid. In some embodiments, it can be desirable to alter one or more codons to encode a conservative mutation, e.g., a similar amino acid with similar chemical structure and properties and/or function as the original amino acid.

In certain embodiments, an optimized polynucleotide sequence encoding an antibody described herein or a fragment thereof (e.g., VL domain or VH domain) can hybridize to an antisense (e.g., complementary) polynucleotide of an unoptimized polynucleotide sequence encoding an antibody described herein or a fragment thereof (e.g., VL domain or VH domain). In specific embodiments, an optimized nucleotide sequence encoding an antibody described herein or a fragment hybridizes under high stringency conditions to antisense polynucleotide of an unoptimized polynucleotide sequence encoding an antibody described herein or a fragment thereof. In some embodiments, an optimized nucleotide sequence encoding an antibody described herein or a fragment thereof hybridizes under high stringency, intermediate or lower stringency hybridization conditions to an antisense polynucleotide of an unoptimized nucleotide sequence encoding an antibody described herein or a fragment thereof. Information regarding hybridization conditions has been described, see, e.g., US 2005/0048549 (e.g., paragraphs 72-73), which is incorporated herein by reference.

The polynucleotides can be obtained, and the nucleotide sequence of the polynucleotides determined, by any method known in the art. Nucleotide sequences encoding antibodies described herein and modified versions of these antibodies can be determined using methods well known in the art, i.e., nucleotide codons known to encode particular amino acids are assembled in such a way to generate a nucleic acid that encodes the antibody. Such a polynucleotide encoding the antibody can be assembled from chemically synthesized oligonucleotides (e.g., as described in Kutmeier G et al., (1994), BioTechniques 17: 242-246), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody or fragment thereof described herein can be generated from nucleic acid from a suitable source (e.g., a hybridoma) using methods well known in the art (e.g., PCR and other molecular cloning methods). For example, PCR amplification using synthetic primers hybridizable to the 3′ and 5′ ends of a known sequence can be performed using genomic DNA obtained from hybridoma cells producing the antibody of interest. Such PCR amplification methods can be used to obtain nucleic acids comprising the sequence encoding the light chain and/or heavy chain of an antibody. Such PCR amplification methods can be used to obtain nucleic acids comprising the sequence encoding the variable light chain region and/or the variable heavy chain region of an antibody. The amplified nucleic acids can be cloned into vectors for expression in host cells and for further cloning, e.g., to generate chimeric and humanized antibodies.

If a clone containing a nucleic acid encoding a particular antibody or fragment thereof is not available, but the sequence of the antibody molecule or fragment thereof is known, a nucleic acid encoding the immunoglobulin or fragment can be chemically synthesized or obtained from a suitable source (e.g., an antibody cDNA library or a cDNA library generated from, or nucleic acid, such as poly A+ RNA, isolated from, any tissue or cells expressing the antibody, such as hybridoma cells selected to express an antibody described herein) by PCR amplification using synthetic primers capable of hybridizing to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR can then be cloned into replicable cloning vectors using any method well known in the art.

DNA encoding recombinant proteins described herein can be readily isolated and sequenced using conventional procedures (e.g., by using oligonucleotide probes that are capable of binding specifically to genes encoding the heavy and light chains of the recombinant proteins). Hybridoma cells can serve as a source of such DNA. Once isolated, the DNA can be placed into expression vectors, which are then transfected into host cells such as E. coli cells, simian COS cells, Chinese hamster ovary (CHO) cells (e.g., CHO cells from the CHO GS System™ (Lonza)), or myeloma cells that do not otherwise produce immunoglobulin protein, to obtain the synthesis of recombinant proteins in the recombinant host cells.

To generate antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a heavy chain constant region, e.g., the human gamma 4 constant region, and the PCR amplified VL domains can be cloned into vectors expressing a light chain constant region, e.g., human kappa or lambda constant regions. In certain embodiments, the vectors for expressing the VH or VL domains comprise an EF-1α promoter, a secretion signal, a cloning site for the variable domain, constant domains, and a selection marker such as neomycin. The VH and VL domains can also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

The DNA also can be modified, e.g., by substituting the coding sequence for human heavy and light chain constant domains in place of the murine sequences, or by covalently joining to the immunoglobulin coding sequence all or part of the coding sequence for a non-immunoglobulin polypeptide.

Also provided are polynucleotides that hybridize under high stringency, intermediate or lower stringency hybridization conditions to polynucleotides that encode an antibody described herein. In specific embodiments, polynucleotides described herein hybridize under high stringency, intermediate or lower stringency hybridization conditions to polynucleotides encoding a VH domain and/or VL domain provided herein.

Hybridization conditions have been described in the art and are known to one of skill in the art. For example, hybridization under stringent conditions can involve hybridization to filter-bound DNA in 6×sodium chloride/sodium citrate (SSC) at about 45° C. followed by one or more washes in 0.2×SSC/0.1% SDS at about 50-65° C.; hybridization under highly stringent conditions can involve hybridization to filter-bound nucleic acid in 6×SSC at about 45° C. followed by one or more washes in 0.1×SSC/0.2% SDS at about 68° C. Hybridization under other stringent hybridization conditions are known to those of skill in the art and have been described, see, e.g., Ausubel F M et al., eds., (1989) Current Protocols in Molecular Biology, Vol. I, Green Publishing Associates, Inc. and John Wiley & Sons, Inc., New York at pages 6.3.1-6.3.6 and 2.10.3.

Other aspects provide recombinant vectors comprising the gene encoding the IL-18-binding protein and the nucleic acid encoding the antigen binding fragment against serum albumin. Still other aspects provide a cell transformed with the vector.

Disclosed herein are nucleic acid molecules encoding a heavy chain region comprising an amino acid sequence having at least 90%, at least 93%, at least 95%, at least 96%, at least 97%, at least 98%, at least 99%, or 100% identity to SEQ ID NO:10. Disclosed herein are nucleic acid molecules encoding a light chain region comprising a nucleotide sequence having at least 90%, at least 93%, at least 95%, at least 96%, at least 97%, at least 98%, at least 99%, or 100% identity to SEQ ID NO:11 or 12.

Disclosed herein are nucleic acid molecules encoding a light chain region comprising an amino acid sequence having at least 90%, at least 93%, at least 95%, at least 96%, at least 97%, at least 98%, at least 99%, or 100% identity to SEQ ID NO:13. Disclosed herein are nucleic acid molecules encoding a light chain region comprising a nucleotide sequence having at least 90%, at least 93%, at least 95%, at least 96%, at least 97%, at least 98%, at least 99%, or 100% identity to SEQ ID NO:14 or 15.

In some embodiments, disclosed herein are nucleic acids, each encoding the light chain region of SEQ ID NO:10 and the heavy chain region of SEQ ID NO:19. In some embodiments, the nucleic acid encoding the light chain region of SEQ ID NO:10 can be represented by SEQ ID NO:11 or SEQ ID NO:12, and the nucleic acid encoding the heavy chain region of SEQ ID NO:19 can be represented by SEQ ID NO:20 or SEQ ID NO:21.

Further disclosed herein are expression vectors comprising:
(a) a promoter,
(b) a first nucleic acid molecule encoding a light chain that binds to serum albumin, and
(c) a second nucleic acid molecule encoding heavy chain and a bioactive effector moiety such as IL-18BP and a linker,
wherein the promoter, the first nucleic acid sequence, and the second nucleic acid molecules are operably linked. The second nucleic acid molecule can encode 1, 2, 3, 4, 5, 6, or more bioactive effector moieties and linkers.

Also disclosed herein are expression vectors comprising:
(a) a promoter and
(b) a nucleic acid molecule encoding a heavy chain variable domain as disclosed herein and a heavy chain constant domain as disclosed herein.

Also disclosed herein are expression vectors comprising:
(a) a promoter and
(b) a nucleic acid molecule encoding a IL18BP as disclosed herein, a heavy chain variable domain as disclosed herein, and a heavy chain constant domain as disclosed herein.

Also disclosed herein are expression vectors comprising:
(a) a promoter and
(b) a nucleic acid molecule encoding a light chain variable domain as disclosed herein and a light chain constant domain as disclosed herein.

Also disclosed herein are expression vectors comprising:
(a) a promoter and
(b) a nucleic acid molecule encoding an IL-18BP as disclosed herein, a light chain variable domain as disclosed herein, and a light chain constant domain as disclosed herein. One, two, three, or more expression vectors or nucleic acid molecules can be expressed to produce the desired recombinant proteins.

In some embodiments, a first nucleic acid molecule or vector comprises a nucleic acid sequence encoding a recombinant protein comprising an antigen binding fragment comprising a heavy chain, wherein the heavy chain comprises a heavy chain variable domain and a heavy chain constant domain, wherein the heavy chain variable domain comprises
(1) a heavy chain complementarity determining domain 1 (CDR1) comprising the amino acid sequence of SYGIS (SEQ ID NO:22),
   a heavy chain complementarity determining domain 2 (CDR2) comprising the amino acid sequence of WINTYSGGTKYAQKFQG (SEQ ID NO:23), and
   a heavy chain complementarity determining domain 3 (CDR3) comprising the amino acid sequence of LGHCQRGICSDALDT (SEQ ID NO:24);
(2) a heavy chain CDR1 comprising the amino acid sequence of SYGIS (SEQ ID NO:22),
   a heavy chain CDR2 comprising the amino acid sequence of RINTYNGNTGYAQRLQG (SEQ ID NO:25), and
   a heavy chain CDR3 comprising the amino acid sequence of LGHCQRGICSDALDT (SEQ ID NO:24);
(3) a heavy chain CDR1 comprising the amino acid sequence of NYGIH (SEQ ID NO:26),
   a heavy chain CDR2 comprising the amino acid sequence of SISYDGSNKYYADSVKG (SEQ ID NO:27), and
   a heavy chain CDR3 comprising the amino acid sequence of DVHYYGSGSYYNAFDI (SEQ ID NO:28);
(4) a heavy chain CDR1 comprising the amino acid sequence of SYAMS (SEQ ID NO:29),
   a heavy chain CDR2 comprising the amino acid sequence of VISHDGGFQYYADSVKG (SEQ ID NO:30), and
   a heavy chain CDR3 comprising the amino acid sequence of AGWLRQYGMDV (SEQ ID NO:31);
(5) a heavy chain CDRlcomprising the amino acid sequence of AYWIA (SEQ ID NO:32),
   a heavy chain CDR2 comprising the amino acid sequence of MIWPPDADARYSPSFQG (SEQ ID NO:33), and
   a heavy chain CDR3 comprising the amino acid sequence of LYSGSYSP (SEQ ID NO:34); or
(6) a heavy chain CDR1 comprising the amino acid sequence of AYSMN (SEQ ID NO:35),
   a heavy chain CDR2 comprising the amino acid sequence of SISSSGRYIHYADSVKG (SEQ ID NO:36), and
   a heavy chain CDR3 comprising the amino acid sequence of ETVMAGKALDY (SEQ ID NO:37).

Disclosed herein is a second nucleic acid molecule or vector comprises a nucleic acid sequence encoding a recombinant protein comprising an antigen binding fragment comprising a light chain, wherein the light chain comprises a light chain variable domain and a light chain constant domain, wherein the light chain variable domain comprises
(7) a light chain CDR1 comprising the amino acid sequence of RASQSISRYLN (SEQ ID NO:38),
   a light chain CDR2 comprising the amino acid sequence of GASRLES (SEQ ID NO:39), and
   a light chain CDR3 comprising the amino acid sequence of QQSDSVPVT (SEQ ID NO:40);
(8) a light chain CDR1 comprising the amino acid sequence of RASQSISSYLN (SEQ ID NO:41),
   a light chain CDR2 comprising the amino acid sequence of AASSLQS (SEQ ID NO:42), and
   a light chain CDR3 comprising the amino acid sequence of QQSYSTPPYT (SEQ ID NO:43);
(9) a light chain CDR1 comprising the amino acid sequence of RASQSIFNYVA (SEQ ID NO:44),
   a light chain CDR2 comprising the amino acid sequence of DASNRAT (SEQ ID NO:45), and
   a light chain CDR3 comprising the amino acid sequence of QQRSKWPPTWT (SEQ ID NO:46);
(10) a light chain CDR1 comprising the amino acid sequence of RASETVSSRQLA (SEQ ID NO:47),
   a light chain CDR2 comprising the amino acid sequence of GASSRAT (SEQ ID NO:48), and
   a light chain CDR3 comprising the amino acid sequence of QQYGSSPRT (SEQ ID NO:49);
(11) a light chain CDR1 comprising the amino acid sequence ofRASQSVSSSSLA (SEQ ID NO:50),
   a light chain CDR2 comprising the amino acid sequence of GASSRAT (SEQ ID NO:48), and
   a light chain CDR3 comprising the amino acid sequence of QKYSSYPLT (SEQ ID NO:51); or
(12) a light chain CDR1 comprising the amino acid sequence of RASQSVGSNLA (SEQ ID NO:52),
   a light chain CDR2 comprising the amino acid sequence of GASTGAT (SEQ ID NO:53), and
   a light chain CDR3 comprising the amino acid sequence of QQYYSFLAKT (SEQ ID NO:54).

For example, the nucleic acid molecule encoding IL-18BP can be linked to the first or second nucleic acid molecule or vector described above.

In other embodiments, the first nucleic acid molecule can comprise a nucleic acid sequence encoding a Fab comprising: a heavy chain variable domain comprising (1) above and a light chain variable domain comprising (7) above; a heavy chain variable domain comprising (2) above and a light chain variable domain comprising (8) above; a heavy chain variable domain comprising (3) above and a light chain variable domain comprising (9) above; a heavy chain variable domain comprising (4) above and a light chain variable domain comprising (10) above; a heavy chain variable domain comprising (5) above and a light chain variable domain comprising (11) above; a heavy chain variable domain comprising (6) above and a light chain variable domain comprising (12) above; or any or all combinations of a heavy chain variable domain and a light chain variable domain described above. In some embodiments, the first nucleic acid molecule comprises a nucleic acid sequence encoding a Fab (SL335) comprising the heavy chain variable domain comprises a heavy chain CDR1 comprising the amino acid sequence of SEQ ID NO:35, a heavy chain CDR2 comprising the amino acid sequence of SEQ ID NO:36, and a heavy chain CDR3 comprising the amino acid sequence of SEQ ID NO:37, and the light chain variable domain comprises a light chain CDR1 comprising the amino acid sequence of SEQ ID NO:52, a light chain CDR2 comprising the amino acid sequence of SEQ ID NO:53, and a light chain CDR3 comprising the amino acid sequence of SEQ ID NO:54. The first or second nucleic acid molecule can encode the IL-18BP.

In other embodiments, a first nucleic acid molecule or vector comprises a nucleic acid sequence encoding a Fab comprising a heavy chain variable domain comprising an amino acid sequence having at least 90%, at least 93%, at least 95%, at least 96%, at least 97%, at least 98%, at least 99%, or 100% identity to SEQ ID NO:55, 56, 57, 58, 59, or 60. In some embodiments, a second nucleic acid molecule or vector comprises a nucleic acid sequence encoding a Fab comprising a light chain variable domain comprising an amino acid sequence having at least 90%, at least 93%, at least 95%, at least 96%, at least 97%, at least 98%, at least 99%, or 100% identity to SEQ ID NO:61, 62, 63, 64, 65, 66, or 67. The nucleic acid molecule encoding IL-18BP can be linked to the first or second nucleic acid molecule or vector.

In some embodiments, a first nucleic acid molecule or vector comprises a nucleic acid sequence encoding a Fab comprising a heavy chain variable domain comprising an amino acid sequence having at least 90%, at least 93%, at least 95%, at least 96%, at least 97%, at least 98%, at least 99%, or 100% identity to SEQ ID NO:55, 56, 57, 58, 59, or 60, and a light chain variable domain comprising an amino acid sequence having at least 90%, at least 93%, at least 95%, at least 96%, at least 97%, at least 98%, at least 99%, or 100% identity to SEQ ID NO:61, 62, 63, 64, 65, or 66 or 67, respectively.

In some embodiments, the first nucleic acid molecule comprises a nucleic acid sequence encoding a Fab (SL335) comprising a heavy chain domain comprising an amino acid sequence having at least 90%, at least 93%, at least 95%, at least 96%, at least 97%, at least 98%, at least 99%, or 100% identity to SEQ ID NO:10 (V_{H}—C_{H1} domain) and a light chain domain comprising an amino acid sequence having at least 90%, at least 93%, at least 95%, at least 96%, at least 97%, at least 98%, at least 99%, or 100% identity to SEQ ID NO:13 (V_{L}—C_{κ} domain).

In some embodiments, the bioactive effector moiety is IL-18BP. In some embodiments, a nucleic acid molecule encodes an IL-18-BP protein comprises an amino acid sequence having at least 90%, at least 93%, at least 95%, at least 96%, at least 97%, at least 98%, at least 99%, or 100% identity to SEQ ID NO:7. In some embodiments, a nucleic acid molecule encoding the IL-18-binding protein comprises a nucleotide sequence having at least 90%, at least 93%, at least 95%, at least 96%, at least 97%, at least 98%, at least 99%, or 100% identity to SEQ ID NO:8 or SEQ ID NO:9. For example, the first nucleic acid molecule can comprise a nucleotide sequence encoding the amino acid sequence having at least 90%, at least 93%, at least 95%, at least 96%, at least 97%, at least 98%, at least 99%, or 100% identity to one or more of SEQ ID NO:7, e.g., SEQ ID NO:8 or 9.

Recombinant expression of an antibody or fragment thereof described herein (e.g., a heavy or light chain of an antibody described herein) that specifically binds to involves construction of an expression vector containing a polynucleotide that encodes the antibody or fragment. Once a polynucleotide encoding an antibody or fragment thereof (e.g., heavy or light chain variable domains) described herein has been obtained, the vector for the production of the antibody molecule can be produced by recombinant DNA technology using techniques well known in the art. Thus, methods for preparing a protein by expressing a polynucleotide containing an antibody or antibody fragment (e.g., light chain or heavy chain) encoding nucleotide sequence are described herein. Methods which are well known to those skilled in the art can be used to construct expression vectors containing antibody or antibody fragment (e.g., light chain or heavy chain) coding sequences and appropriate transcriptional and translational control signals. These methods include, e.g., in vitro recombinant DNA techniques, synthetic techniques, and in vivo genetic recombination. Also provided are replicable vectors comprising a nucleotide sequence encoding an antibody molecule described herein, a heavy or light chain of an antibody, a heavy or light chain variable domain of an antibody or a fragment thereof, or a heavy or light chain CDR, operably linked to a promoter. Such vectors can, e.g., include the nucleotide sequence encoding the constant region of the antibody molecule (see, e.g., WO86/05807 and WO89/01036; and U.S. Pat. No. 5,122,464) and variable domains of the antibody can be cloned into such a vector for expression of the entire heavy, the entire light chain, or both the entire heavy and light chains.

An expression vector can be transferred to a cell (e.g., host cell) by conventional techniques and the resulting cells can then be cultured by conventional techniques to produce an antibody described herein.

A variety of host-expression vector systems can be utilized to express antibody molecules described. Such host-expression systems represent vehicles by which the coding sequences of interest can be produced and subsequently purified, but also represent cells which can, when transformed or transfected with the appropriate nucleotide coding sequences, express an antibody molecule described herein in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems (e.g., green algae such as Chlamydomonas reinhardtii) infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS (e.g., COS1 or COS), CHO, BHK, MDCK, HEK 293, NS0, PER.C6, VERO, CRL7030, HsS78Bst, HeLa, and NIH 3T3, HEK-293T, HepG2, SP210, R1.1, B-W, L-M, BSC1, BSC40, YB/20 and BMT10 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). In some embodiments, cells for expressing antibodies described herein (e.g., an antibody comprising the CDRs of any one of antibodies pab1949 or pab2044) are CHO cells, e.g., CHO cells from the CHO GS System™ (Lonza). In some embodiments, cells for expressing antibodies described herein are human cells, e.g., human cell lines. In some embodiments, a mammalian expression vector is pOptiVEC™ or pcDNA3.3. In some embodiments, bacterial cells such as Escherichia coli, or eukaryotic cells (e.g., mammalian cells), especially for the expression of whole recombinant antibody molecule, are used for the expression of a recombinant antibody molecule. For example, mammalian cells such as Chinese hamster ovary (CHO) cells in conjunction with a vector such as the major intermediate early gene promoter element from human cytomegalovirus is an effective expression system for antibodies (Foecking M K & Hofstetter H (1986) Gene 45: 101-105; and Cockett M I et al., (1990) Biotechnology 8: 662-667). In certain embodiments, antibodies described herein are produced by CHO cells or NS0 cells. In some embodiments, the expression of nucleotide sequences encoding antibodies described herein is regulated by a constitutive promoter, inducible promoter or tissue specific promoter.

In bacterial systems, a number of expression vectors can be advantageously selected depending upon the use intended for the antibody molecule being expressed. For example, when a large quantity of such an antibody is to be produced, for the generation of pharmaceutical compositions of an antibody molecule, vectors which direct the expression of high levels of fusion protein products that are readily purified can be desirable. Such vectors include, but are not limited to, the E. coli expression vector pUR278 (Ruether U & Mueller-Hill B (1983) EMBO J 2: 1791-1794), in which the antibody coding sequence can be ligated individually into the vector in frame with the lac Z coding region so that a fusion protein is produced; pIN vectors (Inouye S & Inouye M (1985) Nuc Acids Res 13: 3101-3109; Van Heeke G & Schuster S M (1989) J Biol Chem 24: 5503-5509); and the like. For example, pGEX vectors can also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In mammalian host cells, a number of viral-based expression systems can be utilized. In cases where an adenovirus is used as an expression vector, the antibody coding sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan J & Shenk T (1984) PNAS 81: 3655-3659). Specific initiation signals can also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see, e.g., Bitter G et al., (1987) Methods Enzymol 153:516-544).

In addition, a host cell strain can be chosen which modulates the expression of the inserted sequences or modifies and processes the gene product in the specific fashion desired. Such modifications (e.g., glycosylation) and processing (e.g., cleavage) of protein products can be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product can be used. Such mammalian host cells include but are not limited to CHO, VERO, BHK, Hela, MDCK, HEK 293, NIH 3T3, W138, BT483, Hs578T, HTB2, BT20 and T47D, NS0 (a murine myeloma cell line that does not endogenously produce any immunoglobulin chains), CRL7030, COS (e.g., COS1 or COS), PER.C6, VERO, HsS78Bst, HEK-293T, HepG2, SP210, R1.1, B-W, L-M, BSC1, BSC40, YB/20, BMT10 and HsS78Bst cells. In certain embodiments, recombinant proteins described herein (e.g., an antibody comprising the CDRs are produced in mammalian cells, such as CHO cells.

In some embodiments, the antibodies described herein have reduced fucose content or no fucose content. Such antibodies can be produced using techniques known one skilled in the art. For example, the antibodies can be expressed in cells deficient or lacking the ability of to fucosylate. In a specific example, cell lines with a knockout of both alleles of α1,6-fucosyltransferase can be used to produce antibodies with reduced fucose content. The Potelligent® system (Lonza) is an example of such a system that can be used to produce antibodies with reduced fucose content.

For long-term, high-yield production of recombinant proteins, stable expression cells can be generated. For example, cell lines which stably express recombinant proteins disclosed herein can be engineered. In specific embodiments, a cell provided herein stably expresses a light chain/light chain variable domain and a heavy chain/heavy chain variable domain which associate to form an antibody described herein (e.g., an antibody comprising the CDRs).

In certain aspects, rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA/polynucleotide, engineered cells can be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which express an antibody described herein or a fragment thereof. Such engineered cell lines can be particularly useful in screening and evaluation of compositions that interact directly or indirectly with the antibody molecule.

A number of selection systems can be used, including but not limited to, the herpes simplex virus thymidine kinase (Wigler M et al., (1977) Cell 11(1): 223-232), hypoxanthineguanine phosphoribosyltransferase (Szybalska E H & Szybalski W (1962) PNAS 48(12): 2026-2034) and adenine phosphoribosyltransferase (Lowy I et al., (1980) Cell 22(3): 817-823) genes can be employed in tk-, hgprt- or aprt-cells, respectively. Also, antimetabolite resistance can be used as the basis of selection for the following genes: dhfr, which confers resistance to methotrexate (Wigler M et al., (1980) PNAS 77(6): 3567-3570; O'Hare K et al., (1981) PNAS 78: 1527-1531); gpt, which confers resistance to mycophenolic acid (Mulligan R C & Berg P (1981) PNAS 78(4): 2072-2076); neo, which confers resistance to the aminoglycoside G-418 (Wu G Y & Wu C H (1991) Biotherapy 3: 87-95; Tolstoshev P (1993) Ann Rev Pharmacol Toxicol 32: 573-596; Mulligan R C (1993) Science 260: 926-932; and Morgan R A & Anderson W F (1993) Ann Rev Biochem 62: 191-217; Nabel G J & Felgner P L (1993) Trends Biotechnol 11(5): 211-215); and hygro, which confers resistance to hygromycin (Santerre R F et al., (1984) Gene 30(1-3): 147-156).

The expression levels of an antibody molecule can be increased by vector amplification (for a review, see Bebbington C R & Hentschel C C G. The use of vectors based on gene amplification for the expression of cloned genes in mammalian cells in DNA cloning, Vol. 3 (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse G F et al., (1983) Mol Cell Biol 3: 257-66).

The host cell can be co-transfected with two or more expression vectors described herein, the first vector encoding a heavy chain derived polypeptide and the second vector encoding a light chain derived polypeptide. The two vectors can contain identical selectable markers which enable equal expression of heavy and light chain polypeptides. The host cells can be co-transfected with different amounts of the two or more expression vectors. For example, host cells can be transfected with any one of the following ratios of a first expression vector and a second expression vector: 1:1, 1:2, 1:3, 1:4, 1:5, 1:6, 1:7, 1:8, 1:9, 1:10, 1:12, 1:15, 1:20, 1:25, 1:30, 1:35, 1:40, 1:45, or 1:50.

Alternatively, a single vector can be used which encodes, and is capable of expressing, both heavy and light chain polypeptides. In such situations, the light chain should be placed before the heavy chain to avoid an excess of toxic free heavy chain (Proudfoot NJ (1986) Nature 322: 562-565; and Kohler G (1980) PNAS 77: 2197-2199). The coding sequences for the heavy and light chains can comprise cDNA or genomic DNA. The expression vector can be monocistronic or multicistronic. A multicistronic nucleic acid construct can encode 2, 3, 4, 5, 6, 7, 8, 9, 10 or more, or in the range of 2-5, 5-10 or 10-20 genes/nucleotide sequences. For example, a bicistronic nucleic acid construct can comprise in the following order a promoter, a first gene (e.g., heavy chain of an antibody described herein), and a second gene and (e.g., light chain of an antibody described herein). In such an expression vector, the transcription of both genes can be driven by the promoter, whereas the translation of the mRNA from the first gene can be by a cap-dependent scanning mechanism and the translation of the mRNA from the second gene can be by a cap-independent mechanism, e.g., by an IRES.

Once an antibody molecule described herein has been produced by recombinant expression, it can be purified by any method known in the art for purification of an immunoglobulin molecule, e.g., by chromatography (e.g., ion exchange, affinity, particularly by affinity for the specific antigen after Protein A, and sizing column chromatography), centrifugation, differential solubility, or by any other standard technique for the purification of proteins. Further, the antibodies described herein can be fused to heterologous polypeptide sequences described herein or otherwise known in the art to facilitate purification.

In specific embodiments, an antibody described herein is isolated or purified. Generally, an isolated antibody is one that is substantially free of other antibodies with different antigenic specificities than the isolated antibody. For example, in some embodiments, a preparation of an antibody described herein is substantially free of cellular material and/or chemical precursors. The language “substantially free of cellular material” includes preparations of an antibody in which the antibody is separated from cellular components of the cells from which it is isolated or recombinantly produced. Thus, an antibody that is substantially free of cellular material includes preparations of antibody having less than about 30%, 20%, 10%, 5%, 2%, 1%, 0.5%, or 0.1% (by dry weight) of heterologous protein (also referred to herein as a “contaminating protein”) and/or variants of an antibody, e.g., different post-translational modified forms of an antibody. When the antibody or fragment is recombinantly produced, it is also generally substantially free of culture medium, i.e., culture medium represents less than about 20%, 10%, 2%, 1%, 0.5%, or 0.1% of the volume of the protein preparation. When the antibody or fragment is produced by chemical synthesis, it is generally substantially free of chemical precursors or other chemicals, i.e., it is separated from chemical precursors or other chemicals which are involved in the synthesis of the protein. Accordingly, such preparations of the antibody or fragment have less than about 30%, 20%, 10%, or 5% (by dry weight) of chemical precursors or compounds other than the antibody or fragment of interest. In some embodiments, antibodies described herein are isolated or purified.

### Compositions

Still other aspects provide compositions, e.g., pharmaceutical compositions, for treating cancers or immune diseases or conditions, the pharmaceutical compositions comprising the recombinant protein as an active ingredient; methods of treating cancers or immune diseases or conditions, the methods comprising administering the composition to a subject; and medical uses of the recombinant protein for preventing or treating cancers or immune diseases or conditions.

For example, the pharmaceutical composition can comprise (a) a pharmaceutically effective amount of the recombinant protein; and (b) a pharmaceutically acceptable carrier.

In some embodiments, the in vivo half-life of the pharmaceutical composition can exhibit a 2- to 20-fold increase, as compared with that of human IL-18BP. The in vivo half-life can exhibit, e.g., about 2.5-fold to about 3.5-fold, about 3.5-fold to about 6-fold increase, about 4-fold to about 6-fold increase, about 4.5-fold to about 6-fold increase, about 5-fold to about 6-fold increase, about 5.5-fold to about 6-fold increase, about 3-fold to about 5.5-fold increase, about 3.5-fold to about 5.5-fold increase, about 4-fold to about 5.5-fold increase, about 4.5-fold to about 5.5-fold increase, about 5-fold to about 5.5-fold increase, about 3-fold to about 5-fold increase, about 3.5-fold to about 5-fold increase, about 4-fold to about 5-fold increase, about 4.5-fold to about 5-fold increase, about 3-fold to about 4.5-fold increase, about 3.5-fold to about 4.5-fold increase, about 4-fold to about 4.5-fold increase, or any fold or ranges of folds derived therefrom, as compared with that of human IL-18BP. In some embodiments, addition, the in vivo half-life of the human IL-18BP can be evaluated after subcutaneous injection of the human IL-18BP.

In some embodiments, the pharmaceutical composition can decrease white blood cell levels in blood. The white blood cells can be, e.g., neutrophils, monocytes, basophils, or a combination thereof. In some embodiments, the decreased white blood cell level can be sustained and maintained until day 20 after administration, until day 15 after administration, until day 12 after administration, until day 10 after administration, until day 8 after administration, until day 7 after administration, or any ranges derived therefrom.

The pharmaceutical composition can be prepared in a unit dosage form or in a multi-dose container by formulating using a pharmaceutically acceptable carrier and/or excipient according to a method that can be easily carried out by a person skilled in the art to which the present disclosure pertains. In this case, the formulation can be in the form of a solution, suspension, or emulsion in an oily or aqueous medium, or in the form of an extract, a suppository, a powder, granules, a tablet, or a capsule, and the formulation can further include a dispersing agent or a stabilizing agent.

Provided herein are compositions comprising a recombinant protein described herein having the desired degree of purity in a physiologically acceptable carrier, excipient or stabilizer (Remington's Pharmaceutical Sciences (1990) Mack Publishing Co., Easton, PA). Also disclosed herein are pharmaceutical compositions comprising a recombinant protein described herein and a pharmaceutically acceptable excipient. Acceptable carriers, excipients, or stabilizers are nontoxic to recipients at the dosages and concentrations employed.

The pharmaceutical composition for preventing or treating immune diseases or cancer according to some aspects can be used after formulating in the form of oral preparations, such as powders, granules, tablets, capsules, suspensions, emulsions, syrups, aerosols, etc., external preparations, suppositories, or sterile injectable preparations according to common methods, and for formulation, the pharmaceutical composition can include an appropriate carrier, excipient, or diluent commonly used in the preparation of pharmaceutical compositions.

The carrier, excipient, or diluent can include various compounds or mixtures, such as lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, mineral oil, etc.

When formulated, it can be prepared using commonly used diluents or excipients, such as fillers, extenders, binders, wetting agents, disintegrants, surfactants, etc.

Solid formulations for oral administration can be prepared by mixing the recombinant fusion protein with at least one excipient, for example, starch, calcium carbonate, sucrose, lactose, gelatin, etc. In addition to simple excipients, lubricants such as magnesium stearate or talc can also be used.

Liquid formulations for oral administration can include suspensions, solutions for internal use, emulsions, syrups, etc. In addition to water and liquid paraffin, which are commonly used simple diluents, various excipients, for example, wetting agents, sweeteners, fragrances, preservatives, etc. can be included.

Formulations for parenteral administration include sterile aqueous solutions, non-aqueous solvents, suspensions, emulsions, lyophilized preparations, and suppositories. The non-aqueous solvents and suspensions can include propylene glycol, polyethylene glycol, vegetable oils such as olive oil, injectable esters such as ethyl oleate, etc. As a suppository base, witepsol, macrogol, tween 61, cacao butter, laurin butter, glycerol gelatin, etc. can be used.

### Uses and Methods

Also disclosed here are methods of treating cancers or immune diseases or conditions in a subject in need thereof, the method comprising administering the pharmaceutical compositions disclosed herein to the subject. The subject can be a human or non-human mammal, such as pets and farm animals. The term “subject” refers to a subject or patient in need of treatment.

As used herein, the term “treating” or “treatment” means all of actions by which symptoms of the disease or condition have improved, been eliminated, or been modified favorably by administering the compositions disclosed herein.

Also disclosed herein are uses of the compositions disclosed herein for the treatment of cancers or immune diseases or conditions in subjects in need thereof. Also disclosed herein are the compositions disclosed herein for use in the treatment of cancers or immune diseases or conditions in subjects in need thereof. Also disclosed herein are the use of the compositions disclosed herein for the manufacture of a medicament for treatment of cancers or immune diseases or conditions in subjects in need thereof.

In some embodiments, the compositions decrease white blood cells in blood of the subject. In some embodiments, the white blood cells are neutrophils, monocytes, basophils, or a combination thereof.

In some embodiments, an elimination half-life (T1/2) of the recombinant proteins disclosed herein is at least about 2-fold greater, at least 2.5-fold, at least about 3-fold greater, at least 3.5-fold, at least about 4-fold greater, at least about 5-fold greater, at least about 7-fold greater, at least about 10-fold greater, or any folds or ranges of fold derived therefrom greater than that of IL-18BP. In some embodiments, the recombinant protein has an elimination half-life (T1/2) of about 8 hrs to about 20 hrs, about 10 hrs to about 18 hours, about 12 hrs to about 15 hrs, or any half-life or ranges derived therefrom. In some embodiments, a Tmax of the recombinant protein is at least about 10% to about 200% higher, about 50% to 100% higher, about 50% to 75% higher, or any % or ranges of % derived therefrom greater than a Tmax of IL-18BP. In some embodiments, a dose of the recombinant protein at about 360 ug/kg of the subject provides a Tmax of about 8 hrs to about 20 hrs, about 10 hrs to about 15 hrs, about 12 hrs to about 14 hrs, or any Tmax or ranges of Tmax derived therefrom. In some embodiments, a Cmax of the recombinant protein is at least about 10% higher, at least about 20% higher, at least about 30% higher, or any % or ranges of % derived therefrom than a Cmax of IL-18BP. In some embodiments, a dose of the recombinant protein at about 360 ug/kg of the subject provides a Cmax of about 700 ng/ml to about 1000 ng/ml, about 750 ng/ml to about 900 ng/ml, about 800 ng/ml to about 850 ng/ml, or any doses or ranges of doses derived therefrom. In some embodiments, an AUClast of the recombinant protein is at least about 2-fold greater, at least 3-fold greater, at least 4-fold greater, at least 5-fold greater, or any fold or ranges of folds derived therefrom than an AUClast of an IL-18BP. In some embodiments, a dose of the recombinant protein at about 360 ug/kg of the subject provides an AUClast of about 8000 hr*ng/ml to about 25000 hr*ng/ml, about 16000 hr*ng/ml to about 22000 hr*ng/ml, about 18000 hr*ng/ml to about 20000 hr*mg/ml, or any concentrations or ranges of concentrations derived therefrom.

In some aspects, presented herein are methods for modulating one or more immune functions or responses in a subject, comprising to a subject in need thereof administering an antibody described herein, or a composition thereof. Disclosed herein are methods for activating, enhancing or inducing one or more immune functions or responses in a subject, comprising to a subject in need thereof administering an antibody or a composition thereof. In some embodiments, presented herein are methods for preventing and/or treating diseases in which it is desirable to activate or enhance one or more immune functions or responses, comprising administering to a subject in need thereof an antibody described herein or a composition thereof. In certain embodiments, presented herein are methods of treating an autoimmune disease or condition comprising administering to a subject in need thereof an antibody or a composition thereof.

In some embodiments, the fusion proteins disclosed herein activate, enhance, or induce one or more immune functions or responses in a subject by at least 99%, at least 98%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10%, or in the range of between 10% to 25%, 25% to 50%, 50% to 75%, or 75% to 95% relative to the immune function in a subject not administered the recombinant protein described herein using assays well known in the art, e.g., ELISPOT, ELISA, and cell proliferation assays.

Pharmaceutical compositions described herein can be useful in enhancing, inducing, or activating the activities of the recombinant proteins disclosed herein and treating a disease or condition.

The compositions to be used for in vivo administration can be sterile. This is readily accomplished by filtration through, e.g., sterile filtration membranes.

Still other aspects provide a pharmaceutical composition for preventing or treating immune diseases, the pharmaceutical composition including the recombinant fusion protein as an active ingredient. Still other aspects provide a method of preventing or treating immune diseases, the method including administering the recombinant fusion protein to an individual. Specific details of the recombinant fusion protein are as described above.

The immune diseases can be inflammatory diseases or autoimmune diseases. The inflammatory diseases can be, for example, adult-onset Still's disease, systemic juvenile idiopathic arthritis, macrophage activation syndrome, hemophagocytic lymphohistiocytosis, atopic dermatitis, psoriasis, dermatitis, allergy, arthritis, rhinitis, otitis media, sore throat, tonsillitis, cystitis, nephritis, pelvic inflammation, Crohn's disease, ulcerative colitis, ankylosing spondylitis, systemic lupus erythematosus (SLE), asthma, edema, delayed allergy (type IV allergy), transplant rejection, graft-versus-host disease, autoimmune encephalomyelitis, multiple sclerosis, inflammatory bowel disease, cystic fibrosis, diabetic retinopathy, ischemic-reperfusion injury, vascular restenosis, glomerulonephritis, gastrointestinal allergy, etc. Further, the immune diseases can be, for example, rheumatoid arthritis, Sjogren's syndrome, systemic sclerosis, polymyositis, systemic angitis, mixed connective tissue disease, Crohn's disease, Hashimoto's disease, Grave's disease, Goodpasture's syndrome, Guillain-Barre syndrome, idiopathic thrombocytopenic purpura, irritable bowel syndrome, myasthenia gravis, hypnolepsy, pemphigus vulgaris, pernicious anemia, primary biliary cirrhosis, ulcerative colitis, vasculitis, Wegener's granulomatosis, psoriasis, etc.

In some embodiments, the pharmaceutical composition can be a pharmaceutical composition for preventing or treating adult-onset still's disease, the pharmaceutical composition including the recombinant fusion protein as an active ingredient. Adult-onset still's disease is a multifactorial systemic autoinflammatory disease that has similar symptoms to systemic juvenile idiopathic arthritis, and is an inflammatory disease that occurs in adults, but the exact pathogenic mechanisms of the disease remain unknown. The adult-onset still's disease is characterized in that a concentration of interleukin-18 in the blood is increased and a concentration of IL-18-binding protein, which is an antagonist in vivo, is down-regulated. Meanwhile, it was reported that more than 50% of patients with adult-onset still's disease who received a recombinant IL-18BP drug at doses of 80 mg/head and 160 mg/head, respectively, responded to the drug. In addition, clinical trials reported that the drug has a half-life of about 30 hours to about 40 hours in humans and is effective when administered three times a week. Thus, there is a problem in that the drug needs to be frequently administered, as a formulation for subcutaneous injection, to patients. In one exemplary embodiment, it was confirmed that the recombinant fusion protein exhibited a half-life about 3.5 times extended in rats, as compared with the recombinant IL-18BP, and had the same and similar activity even when a small dose is administered. Therefore, the recombinant fusion protein can be effectively used for the treatment of adult-onset still's disease.

Still other aspects provide a pharmaceutical composition for preventing or treating cancer, the pharmaceutical composition including the recombinant fusion protein as an active ingredient. Still other aspects provide a method of preventing or treating cancer, the method including administering the recombinant fusion protein to an individual. Specific details of the recombinant fusion protein are as described above.

The cancer can be, for example, multiple myeloma, lung cancer, liver cancer, stomach cancer, colorectal cancer, colon cancer, skin cancer, bladder cancer, prostate cancer, breast cancer, ovarian cancer, cervical cancer, thyroid cancer, kidney cancer, fibrosarcoma, melanoma, blood cancer, etc.

### Routes of Administration & Dosages

The pharmaceutical compositions of the present disclosure can be administered to a subject through a variety of administration routes including oral, transcutaneous, subcutaneous, intravenous, and intramuscular administration routes.

The amount of a recombinant protein or composition disclosed herein that will be effective in the treatment and/or prevention of a condition will depend on the nature of the disease and can be determined by standard clinical techniques.

In the present disclosure, the amount of the recombinant protein disclosed herein that is actually administered is determined in light of various relevant factors including the disease to be treated, a selected route of administration, the age, sex and body weight of a patient, and severity of the disease, and the type of a bioactive polypeptide as an active ingredient. Since the recombinant protein of the present disclosure has excellent sustainability in blood, the number and frequency of administration of the peptide preparations comprising the recombinant protein of the present disclosure can be noticeably reduced.

The pharmaceutical composition is administered in a pharmaceutically effective amount. As used herein, the “pharmaceutically effective amount” or “effective amount” in the context of the administration of a therapy to a subject refers to the amount of a therapy that achieves a desired prophylactic or therapeutic effect. An effective dose level can be determined depending on factors including a patient's disease type, severity, drug activity, drug sensitivity, administration time, administration route and excretion ratio, treatment period, and co-administered drugs, and other factors well known in the medical field. The pharmaceutical composition can be administered as a single therapeutic agent or in combination with other therapeutic drugs, and can be administered with existing therapeutic drugs simultaneously, separately, or sequentially, once or in a few divided doses. It is important to administer the composition in a minimum amount sufficient to obtain the maximum effect without any side effects, considering all the factors, and this amount can be easily determined by those skilled in the art. As used herein, the term “pharmaceutically effective amount” refers to an amount sufficient to treat the cancers or immune diseases or conditions.

An appropriate dosage of the pharmaceutical composition for preventing or treating immune diseases or cancer according to some aspects varies depending on a patient's conditions, body weight, disease severity, drug formulation, administration route and period, but can be appropriately selected by those skilled in the art. However, for desirable effects, the pharmaceutical composition can be administered at a daily dose of 0.0001 mg/kg to 2,000 mg/kg, and specifically, 0.001 mg/kg to 2,000 mg/kg. Administration can be performed once a day, or in several divided doses.

The precise dose to be employed in a composition will also depend on the route of administration, and the seriousness of the disease, and should be decided according to the judgment of the practitioner and each subject's circumstances. For example, effective doses can also vary depending upon means of administration, target site, physiological state of the patient (including age, body weight and health), other medications administered, or whether treatment is prophylactic or therapeutic. Usually, the patient is a human but can be a non-human, such as pets, e.g., dogs and cats. Treatment dosages are optimally titrated to optimize safety and efficacy.

In certain embodiments, an in vitro assay is employed to help identify optimal dosage ranges. Effective doses can be extrapolated from dose response curves derived from in vitro or animal model test systems.

In some embodiments, the recombinant fusion protein can be administered at a dose of 0.001 mg/kg to 2,000 mg/kg. For example, the recombinant fusion protein can be administered at a dose of 0.001 mg/kg to 0.01 mg/kg, 0.1 mg/kg to 1 mg/kg, 1.5 mg/kg to 2 mg/kg, 4 mg/kg to 10 mg/kg, 15 mg/kg to 20 mg/kg, 30 mg/kg to 40 mg/kg, 60 mg/kg to 80 mg/kg, 100 mg/kg to 200 mg/kg, or any dose or ranges of doses derived therefrom.

The pharmaceutical composition for preventing or treating immune diseases according to some aspects can be administered to mammals, such as rats, mice, livestock, humans, etc., via various routes. All modes of administration can be contemplated, for example, by oral, rectal or intravenous, intramuscular, subcutaneous, or intradural administration, or intracerebroventricular injection.

The pharmaceutical composition can be orally or parenterally administered. Specifically, the pharmaceutical composition can be parenterally administered, and in this case, it can be administered by intravenous injection, subcutaneous injection, intramuscular injection, intraperitoneal injection, endothelial administration, topical administration, intranasal administration, intrapulmonary administration, and rectal administration. In some embodiments, it can be administered in the form of subcutaneous injection. When orally administered, a protein or peptide is digested, and therefore, it is required to formulate an oral composition by coating the active ingredient or protecting it from degradation in the stomach. In addition, the pharmaceutical composition can be administered by any device capable of delivering an active substance to target cells.

Still other aspects provide a health functional food composition for preventing or improving immune diseases, the health functional food composition including the recombinant fusion protein as an active ingredient. Still other aspects provide a health functional food composition for preventing or improving cancer, the health functional food composition including the recombinant fusion protein as an active ingredient. Specific details of the recombinant fusion protein, immune diseases, and cancer are as described above.

With regard to the health functional food composition for preventing or improving immune diseases or conditions or cancers, the recombinant fusion protein can be added as it is or can be used with other food or food ingredients, when the recombinant fusion protein is used as an additive for the health functional food and can be used appropriately according to a common method. A mixing amount of the active ingredient can be appropriately determined according to each purpose of use, such as prevention, health, treatment, etc.

The formulation of the health functional food can be in the form of powders, granules, pills, tablets, and capsules, as well as in the form of general foods or beverages.

The type of food is not particularly limited, and examples of the food, to which the substance can be added, can include meats, sausages, bread, chocolates, candies, snacks, confectionery, pizza, ramen, other noodles, gums, dairy products including ice cream, various soups, beverages, teas, drinks, alcoholic beverages, vitamin complexes, etc., and can include all foods in common sense.

In general, in the preparation of foods or beverages, the recombinant fusion protein can be added in an amount of 15 parts by weight or less, and specifically, 10 parts by weight or less, based on 100 parts by weight of the raw material. However, in the case of long-term intake for the purpose of health and hygiene or for the purpose of health control, the amount can be adjusted to be below the above range. Further, the present disclosure has no problem in terms of safety, because a fraction from a natural product is used. Accordingly, the amount can be above the range.

Among the health functional foods, beverages can include various flavoring agents or natural carbohydrates as additional ingredients, like in common beverages. The above-mentioned natural carbohydrates can include monosaccharides such as glucose and fructose, disaccharides such as maltose and sucrose, polysaccharides such as dextrin and cyclodextrin, and sugar alcohols such as xylitol, sorbitol, erythritol, etc. As a sweetener, natural sweeteners such as taumatin and stevia extract, synthetic sweeteners such as saccharin and aspartame, etc. can be used. A proportion of the natural carbohydrate can be about 0.01 g to 0.04 g, and specifically, about 0.02 g to 0.03 g per 100 mL of the beverage according to the present disclosure.

In addition, the health functional food composition for preventing or improving immune diseases or cancer according to some aspects can include various nutrients, vitamins, electrolytes, flavoring agents, coloring agents, pectic acid and salts thereof, alginic acid and salts thereof, organic acids, protective colloidal thickeners, pH adjusters, stabilizers, preservatives, glycerin, alcohols, and carbonating agents used in carbonated beverages. In addition, the composition for improving immune diseases or cancer of the present disclosure can include fruit flesh for the preparation of natural fruit juice, fruit juice beverages, and vegetable beverages. These components can be used independently or in a mixture. A proportion of these additives is not limited but is generally selected from the range of 0.01 part by weight to 0.1 part by weight, relative to 100 parts by weight of the health functional food composition.

As described above, the recombinant fusion protein can exhibit, e.g., a half-life about 3.5 times extended in rats, as compared with the human recombinant IL-18BP, and exhibits a biological activity at a similar level to that of IL-18BP not fused to SL335. Therefore, since the recombinant fusion protein can exhibit similar efficacy even with less frequency of administration, patients can be administered with the drug at more convenient intervals.

### Kits

Provided herein are kits comprising one or more recombinant proteins described herein or conjugates thereof. Disclosed herein are kits comprising the compositions disclosed herein and labels comprising instructions for uses thereof. In some embodiments, provided herein is a pharmaceutical pack or kit comprising one or more containers filled with one or more of the ingredients of the pharmaceutical compositions described herein, such as one or more recombinant proteins provided herein. In some embodiments, the kits contain a pharmaceutical composition described herein and any prophylactic or therapeutic agent, such as those described herein. Optionally associated with such container(s) can be a notice in the form prescribed by a governmental agency regulating the manufacture, use or sale of pharmaceuticals or biological products, which notice reflects approval by the agency of manufacture, use or sale for human administration. Also provided herein are kits that can be used in the above methods. In some embodiments, a kit comprises a recombinant protein described herein, e.g., a purified recombinant protein, in one or more containers. In some embodiments, kits described herein contain a substantially isolated antigen(s) (e.g., human serum albumin) that can be used as a control. In other embodiments, the kits described herein further comprise a control antibody which does not react with a serum albumin antigen. In other embodiments, kits described herein contain one or more elements for detecting the binding of a recombinant protein to a serum albumin antigen (e.g., the recombinant protein can be conjugated to a detectable substrate such as a fluorescent compound, an enzymatic substrate, a radioactive compound or a luminescent compound, or a second antibody which recognizes the first antibody can be conjugated to a detectable substrate). In specific embodiments, a kit provided herein can include a recombinantly produced or chemically synthesized serum albumin antigen. The serum albumin antigen provided in the kit can also be attached to a solid support. In some embodiments, the detecting means of the above-described kits include a solid support to which a serum albumin antigen is attached. Such kits can also include a non-attached reporter-labeled anti-human antibody or anti-mouse/rat antibody. In binding of the antibody to the serum albumin, the antigen can be detected by binding of the said reporter-labeled antibody.

### ADVANTAGEOUS EFFECTS OF DISCLOSURE

A recombinant fusion protein according to some aspects is prepared by fusing an IL-18-binding protein with an anti-serum albumin antibody, and there is an advantage in that the recombinant fusion protein has a relatively long administration cycle due to the increased half-life in the body. Further, since the recombinant fusion protein has low immunogenicity and does not cause side effects in vivo, it can be effectively used for the treatment of various immune diseases including adult-onset still's disease.

Hereinafter, exemplary embodiments will be provided for better understanding of the present disclosure. However, the following exemplary embodiments are provided only for understanding the present disclosure more easily, but the content of the present disclosure is not limited by the following exemplary embodiments.

### EXAMPLES

### Example 1. Preparation of Recombinant Fusion Protein Including Interleukin-18-Binding Protein and Antigen Binding Fragment Against Serum Albumin

### 1-1. Preparation of Vector Expressing Interleukin-18-Binding Protein and Vector Expressing Antigen Binding Fragment Against Serum Albumin

A recombinant fusion protein including an interleukin IL-18-binding protein (IL-18BP) and an antibody fragment binding to serum albumin was prepared. A human interleukin-18-binding protein (hIL-18BP) gene needed in experiments was synthesized by Cosmogenetech co, Ltd. (South Korea). A Fab antibody fragment (SL335) binding to human serum albumin was selected from human naïve antibody libraries, and heavy chain and light chain genes were synthesized by ATUM (Newark, California).

A recombinant gene (SL335H-Linker-hIL18BP), in which hIL-18BP was linked to the C-terminus of SL335 heavy chain via a peptide linker (GSAPAPGS; SEQ ID NO:16), was prepared. In detail, the SL335H-Linker-hIL18BP gene was amplified using primers represented by SEQ ID NOS:1 to 4 of Table 1 below under conditions of 30 cycles of 95° C. for 1 minute, 60° C. for 1 minute, and 72° C. for 1 minute. Thereafter, the amplified recombinant gene and pD2535NT to be used as an expression vector were treated with BbsI (Takara, Japan) restriction enzyme, respectively to digest the BbsI site, and then treated with T4 DNA ligase, and inserted into each vector. Meanwhile, an SL335 light chain gene was inserted into a pD2359 vector.

TABLE 1

| No. | Name | F/R | 5′ to 3′ |
|---|---|---|---|
| SEQ ID | SL335 H Xba1 | Forward | gatcaactctagagccac |
| NO: 1 | Kozak | | catggagtggtcctgggt |
| SEQ ID | GS vector | Reverse | gtgctacctggggcaggg |
| NO: 2 | GSAP linker | | gctgacccg |
| SEQ ID | GS vector | Forward | cgggtcagcccctgcccc |
| NO: 3 | GSAP linker | | aggtagcac |
| SEQ ID | huIL18BP | Reverse | cgcgaagacgcttttaga |
| NO: 4 | Not1, Bbs1 | | gcggccgcgtctacctac |
| | | | ccttgctgctg |
| SEQ ID | huIL18BP | Forward | ggctgagcggggtggagg |
| NO: 5 | only | | ggacacctgtgtcccaga |
| | | | ccacaacagccgctacag |
| | | | c |
| SEQ ID | huIL18BP | Reverse | atcggcggccgcgaagac |
| NO: 6 | his8 | | gcttttagatcagtggtg |
| | BbsI NotI | | gtggtgatggtggtggtg |
| | | | ccccccttgctgctgtgg |
| | | | gctagaatggctacttgg |

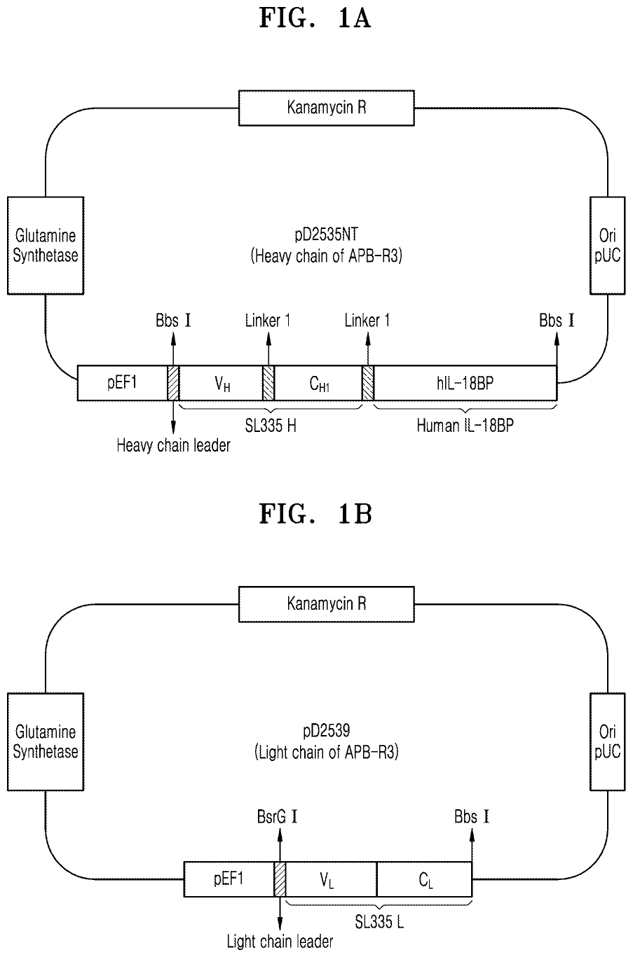

FIGS. 1A and 1B show heavy chain (FIG. 1A) and light chain (FIG. 1B) expression vectors for the preparation of the recombinant fusion protein according to an aspect. As shown in FIG. 1, in the heavy chain, human recombinant IL-18BP was linked to the C-terminus of SL335H via the peptide linker.

### 1-2. Preparation of Transient Expression Cells

ExpiCHO-S™ cells (Thermo Fisher scientific) were suspended in a 125 ml culture flask containing an expression medium (ExpiCHO expression media, Thermo Fisher Scientific), and then cultured in a shaking incubator under conditions of 37° C., 140 rpm, 5% CO₂, and 80% humidity. Thereafter, to prepare transient expression cells, the cultured cells were dispensed at a density of 6.0×10⁶ cells/ml, and plasmid vectors (pD2535NT, pD2539), into which the heavy chain and light chain genes prepared in Example 1-1 were respectively inserted, were transfected into the dispensed cells. Then, the cells were cultured in the shaking incubator under the same conditions as above for 16 hours, and immediately treated with an ExpiCHO feed and enhancer. On day 3 of culture, the cells were further treated with the ExpiCHO feed, and cultured for 8 days at an incubator temperature set to 32° C. After the culture was completed, the harvested culture medium was centrifuged at 4,000 rpm and 4° C. for 15 minutes to separate the cells and the culture medium. Then, the culture medium was passed through a 0.2 μm filter paper to remove impurities.

### 1-3. Preparation of Stable Cell Line

A stable cell line was prepared using HD-BIOP3 GS null CHO-K1 cells (Horizon Discovery). In detail, cells were dispensed at a density of 3.0×10⁵ cells/ml in a CD FortiCHO (Thermo Fisher Scientific) medium containing 4 mM of L-glutamine, followed by seed culture for one day in a shaking incubator under conditions of 37° C., 5% CO₂, and 80% or more humidity. For transfection, cells were aliquoted at a density of 1.0×10⁶ cells/ml, and each plasmid vector (pD2535NT, pD2539) prepared in Example 1-1 was transfected into the dispensed cells using an OptiPRO SFM medium and a Freestyle max reagent (Invitrogen, Carlsbad, California), and then cultured for 2 days under conditions of 37° C., 5% CO₂, and 80% or more humidity. Thereafter, to perform stable pool selection, the medium was replaced with a CD FortiCHO medium without L-glutamine by centrifugation, and 50 μM of methionine sulfoximine (MSX) (Sigma-Aldrich, St. Louis, Missouri) and 10 μg/ml of puromycin (Thermo Fisher Scientific) were treated every two days to remove cells not transfected with the vector. Thereafter, the medium was replaced by a CD FortiCHO medium containing both MSX and puromycin every 7 to 10 days using a centrifuge, and the number of cells was maintained at 5.0×10⁵ cells/ml each time and cultured for 21 days. Thereafter, when viability was recovered at 90% or more, a stock was prepared at 1.0×10⁷ cells/ml.

### 1-4. Isolation and Purification of APB-R3 Protein

The protein sample present in the culture medium of Example 1-3 was purified by sequentially performing affinity chromatography (AC), cation exchange chromatography (CEX), and anion exchange chromatography (AEX). In detail, affinity chromatography was performed at a flow rate of 8 ml/min, and cation exchange chromatography was performed at a flow rate of 2 ml/min. Then, the proteins purified by the cation exchange chromatography were passed through anion exchange chromatography, and the proteins not bound to resins were recovered. The protein purified by the above method was named APB-R3.

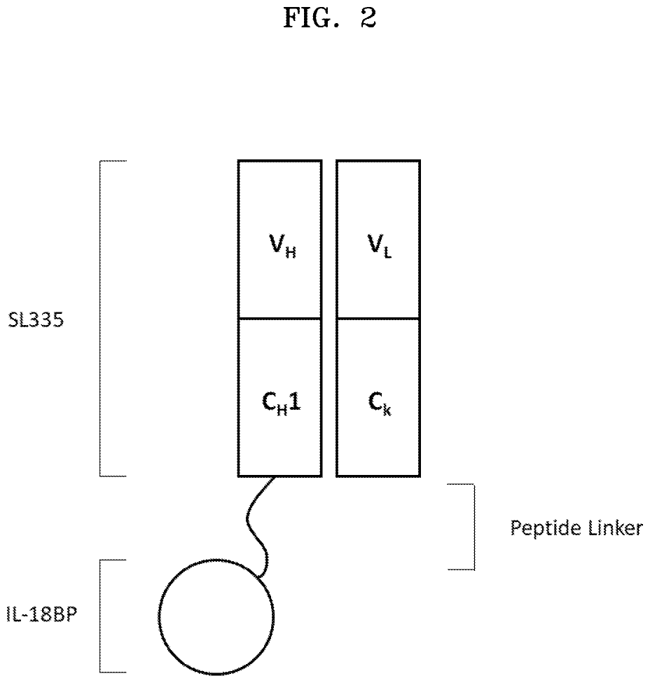

FIG. 2 shows a structure of the APB-R3 protein according to an aspect. As shown in FIG. 2, APB-R3 was confirmed to have a structure, in which SL335 which is a human Fab fragment specifically binding to serum albumin, and TL-18BP were linked via a peptide linker, and the heavy chain (SL335H-peptide linker-IL-18BP) and the light chain (SL335L) were noncovalently linked. It was also confirmed that SL335 consists of human V_{H}-C_{H1} (SL335H) and VL-C_{κ} (SL335L). Meanwhile, SL335 which is a sequence selected from human naïve antibody libraries, is expected to have very low immunogenicity, because the sequence is very similar to the original human antibody sequence.

### Comparative Example 1. Preparation of Recombinant Human Interleukin-18-Binding Protein

A recombinant gene, in which a His-tag (HHHHHHBH; SEQ ID NO:85) was linked to the C terminus of hIL-18BP, was prepared. In detail, hIL18BP-his8 was amplified under the same conditions as in Example 1-1 using primers represented by SEQ ID NOS: 5 and 6 of Table 1. Then, the product was inserted into an expression vector in the same manner as in Example 1-1, and then a recombinant human IL-18-binding protein was prepared by the methods of Examples 1-2 to 1-4. Then, the protein was purified using HiTrap IMAC HP (GE Healthcare) and AKTA pure 150 L equipment.

### Experimental Example 1. Molecular Characteristics of APB-R3 Protein

### 1-1. Examination of Size

SDS-PAGE was performed to examine the sizes of the proteins prepared in Example 1 and Comparative Example 1. In detail, protein samples were prepared using a non-reducing 4×SDS sample buffer (Thermo Fisher Scientific) and 2-mercaptoethanol under reducing and non-reducing conditions. For the non-reducing conditions, samples which were heated at 100° C. for 5 minutes or not heated were prepared to compare the shape and size of the proteins by heating. A protein size marker (SMOBio, Taiwan) was prepared to compare the sizes of proteins under the respective conditions. The prepared protein samples were loaded into a Mini-protein TGX precast gel, 4-15%, 15-well (Bio-Rad) in an amount of 1 μg or 2 μg per well, followed by electrophoresis in a tris-glycine SDS running buffer at 150 V for 1 hour. After the electrophoresis was completed, the SDS-PAGE gel was reacted with an EZ-Gel staining solution (DoGenBio, South Korea) to perform staining for 1 hour, followed by destaining in distilled water for one day.

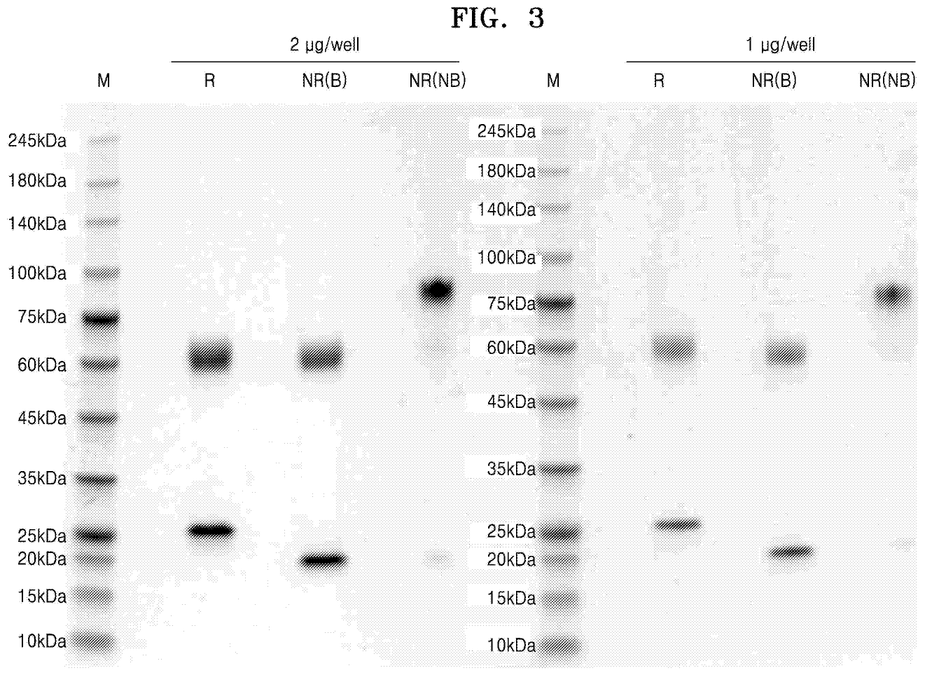

FIG. 3 shows SDS-PAGE results of analyzing the size of the APB-R3 protein in an amount of 1 μg/well and 2 μg/well under reducing (R), non-reducing and boiled (NR(B)), and non-reducing and non-boiled (NR(NB)) conditions. As shown in FIG. 3, SL335H-IL18BP heavy chain protein under reducing conditions and non-reducing and boiled conditions for 5 minutes exhibited a protein band at about 60 kDa, which is higher than a theoretical size of 41.905 kDa, due to additional N-linked and O-linked glycans, and SL335L light chain exhibited a protein band at 20 kDa to 25 kDa similar to a theoretical size (23.311 kDa). In contrast, under non-reducing and non-boiled conditions, a protein band corresponding to an intact form of APB-R3, in which the heavy chain and the light chain were bound, was observed with high purity at 75 kDa to 100 kDa, and protein bands, each corresponding to unbound heavy and light chains, were weakly detected.

### 1-2. Examination of Purity

SE-HPLC was performed to measure purity of the APB-R3 protein purified in Example 1-4. First, a TSKgel UltraSW Aggregate 7.8×300 mm (Tosoh Bioscience, Japan) column and 1260 infinity II LC system (Agilent Technologies, Santa Clara, California) HPLC equipment were equilibrated with 20 mM citric acid at pH 5.5 buffer. Analytical samples were prepared by diluting with 20 mM citric acid at pH 5.5 buffer, and ˜25 μg of the sample was loaded onto the column. SE-HPLC analysis was performed at a flow rate of 0.7 ml/min and a maximum pressure limit of 120 bar for 30 minutes, and purity was measured at A280 nm wavelength.

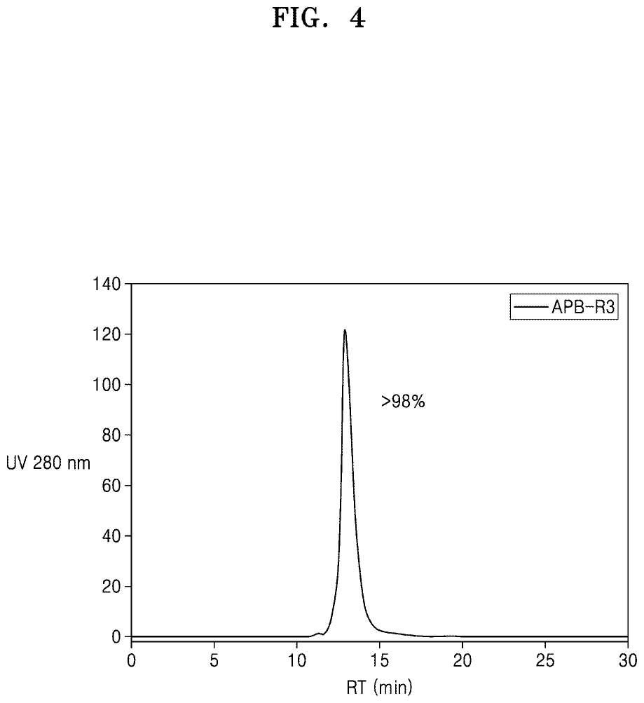

FIG. 4 shows SEC-HPLC results of analyzing purity of the APB-R3 protein according to an aspect. As shown in FIG. 4, it was confirmed that the APB-R3 protein had purity of 98% or more.

### 1-3. Examination of Isoelectric Point

In order to measure an isoelectric point (pI) of the APB-R3 protein purified in Example 1-4, isoelectric focusing analysis (IEF) was performed. In detail, a pH3-10 IEF gel (Koma gel) was used, and 3 μg, 5 μg, and 10 μg of samples were loaded, and the conditions were at 100 V for 1 hour, at 200 V for 1 hour, and at 500 V for 1 hour. The protein was fixed using 12% trichloroacetic acid (TCA) and stained with coomassie brilliant blue (CBB). Thereafter, pI of the protein was analyzed using ImageMaster™ 2D Platinum (GE Healthcare, ver. 5.0).

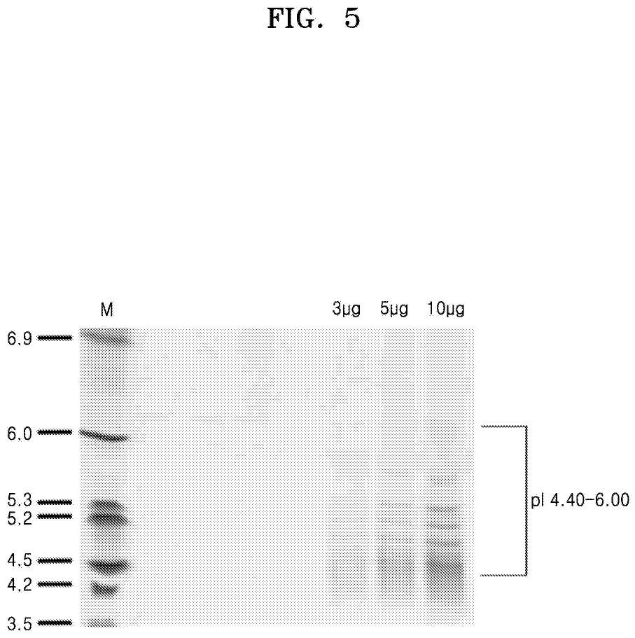

FIG. 5 shows results of analyzing an isoelectric point of the APB-R3 protein according to an aspect. As shown in FIG. 5, a band was observed at pI of 4.40 to 6.00.

### 1-4. Examination of Molecular Weight

Intact mass spectrometry analysis was performed to measure a molecular weight of the APB-R3 protein of Example 1. In detail, intact mass spectrometry analysis was performed under reduced conditions (20 mM DTT, 37° C.) using Dionex UHPLC (Thermo Fisher Scientific) and Q-TOF 5600+MS/MS system (AB SCIEX, CA, USA). The molecular weight was measured in a mobile phase of acetonitrile (ACN; J. T. Baker) at a flow rate of 0.3 ml/min using Acquity UPLC® BEH130 C4, 1.7 μm column, and the results are shown in Table 2 below.

TABLE 2
Results of expected and measured molecular weight

| Sample | | Measured weight (m/z) |
|---|---|---|
| APB-R3 | light chain | 23.306 |
| | heavy chain | 46.611 |

As shown in Table 2, it was confirmed that molecular weights of the light chain (SL335L) and the heavy chain (SL335H-IL18BP) of the APB-R3 protein were 23.306 kDa and 46.611 kDa, respectively.

### Experimental Example 2. Evaluation of Biological Activity of APB-R3 Protein

### 2-1. Examination of IL-18 Inhibition Degree (1)

To evaluate biological functions of the APB-R3 protein purified in Example 1-4, IL-18 inhibition degree of the protein was examined using a human KG-1 cell line (ATCC, CCL-246) expressing IFN-γ in response to IL-18. First, the protein sample of Example 1 was prepared by diluting the sample with a PBS buffer supplemented with 0.3% bovine serum albumin (BSA). Thereafter, each sample was treated with a recombinant human IL-18 protein (R&D Systems) and allowed to react for 1 hour in a 37° C. incubator. Thereafter, 1.3×10⁶ cells/ml of KG-1 cells were prepared in an EIDM medium containing recombinant TNF-α (BioLegend, San Diego, California), and then dispensed into the protein mixture, in which the reaction was completed. The mixture of the cells and proteins were reacted for 23 hours in an incubator under 37° C. and 5% CO₂ conditions, and then the supernatant and cells were separated using a centrifuge. The separated supernatant was analyzed to measure the amount of IFN-γ secreted from KG-1 cells by recombinant IL-18. The concentration of IFN-γ secreted in the supernatant was analyzed using ELISA MAX™ Deluxe Set human IFN-γ (BioLegend) and performed according to the standard experimental method specified in the product. Comparative Example 1 and SL335 protein were analyzed in the same manner.

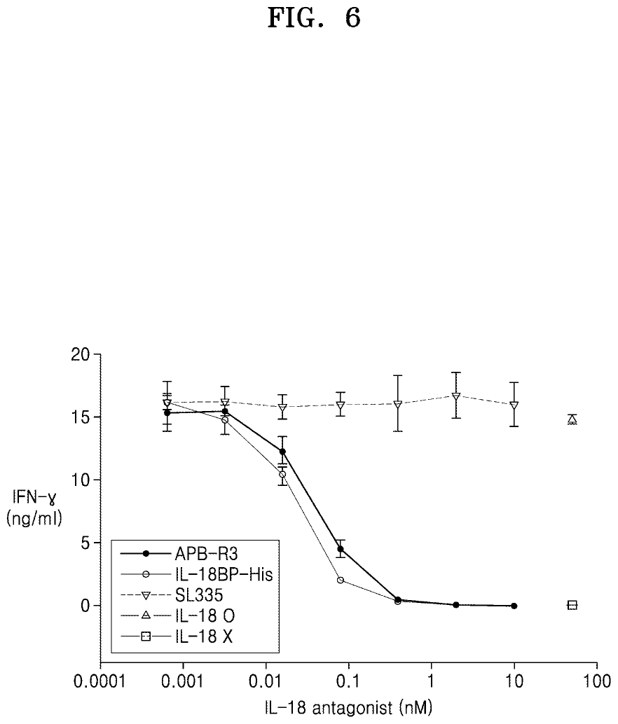

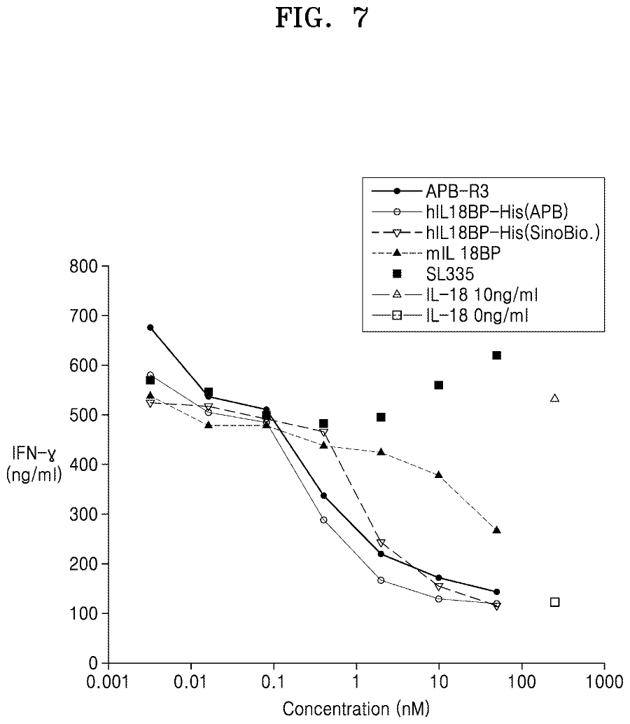

FIG. 6 shows a graph showing IL-18 inhibition in the KG-1 cell line by the APB-R3 protein according to an aspect. As shown in FIG. 6, KG-I cells produced and expressed IFN-γ in an IL-18 concentration-dependent manner, and the APB-R3 protein inhibited IFN-γ production in a concentration-dependent manner, similar to IL-18BP-His protein. In addition, IC₅₀ of APB-R3 was 0.0419 nM, and IC₅₀ of IL-18BP-His was 0.0240 nM, indicating that human IL-18BP fused to SL335 maintained its intact biological property.

### 2-2. Examination of IL-18 Inhibition Degree (2)

To evaluate biological functions of the APB-R3 protein purified in Example 1-4, IL-18 inhibition degree of APB-R3 was examined using Naïve CD4+ T cells isolated from C57BL/6 mouse (Orient Bio). First, anti-CD3 (Biolegend, clone 145-2C11) diluted at a concentration of 5 μg/ml using a PBS buffer was dispensed in an amount of 50 μl in each well of a 96-well culture plate (Corning, 3596), and then coated at 4° C. for 16 hours. The plate was washed twice with 200 l/well of PBS before adding the cells. Prior to treatment of the isolated Naïve CD4+ T cells with the sample, the following pretreatment was performed. A recombinant mouse IL-18 protein (R&D Systems) and a recombinant mouse IL-12 protein (PeproTech) were prepared at a concentration of 10 ng/ml by diluting with an RPMI1640 (Gibco) medium containing 10% fetal bovine serum (FBS) (Gemini bio), 50 μM 2-mercaptoethanol (Gibco), 10 mM HEPES (Gibco), and 5 μg/ml gentamycin (Gibco), and then mixed with APB-R3 protein samples serially diluted and allowed to react in a 37° C. incubator for 1 hour. Then, the reacted samples were treated with 1.0×10⁵ cells/ml of CD4+ T cells and allowed to react for 48 hours in an incubator under 37° C. and 5% CO₂ conditions. To measure the amount of IFN-γ secreted from CD4+ T cells by the recombinant mouse IL-18, the separated supernatant was analyzed using ELISA MAX™ Deluxe Set mouse IFN-γ (BioLegend) and performed according to the standard experimental method specified in the product. Comparative Example 1, recombinant mouse IL-18BP (R&D System), and SL335 protein were analyzed in the same manner.

FIG. 7 shows a graph showing IL-18 inhibition in mouse CD4+ T cells by the APB-R3 protein according to an aspect. As shown in FIG. 7, mouse CD4+ T cells produced and expressed INF-7 in an IL-18 concentration-dependent manner, and the APB-R3 protein inhibited IFN-γ production in a concentration-dependent manner, similar to human IL-18BP-His protein. In contrast, it was confirmed that mouse IL-18BP inhibits IFN-γ production in a concentration-dependent manner, but its inhibitory activity was lower than that of APB-R3.

### Experimental Example 3. Pharmacokinetic Evaluation of APB-R3 Protein

Absorption, distribution, in vivo changes, and excretion of the APB-R3 protein prepared in Example 1 were examined through pharmacokinetics. In detail, after breeding 20 healthy male rats from Koatech (South Korea), single subcutaneous administration of the protein of Example 1 was performed for five mice each at a dose of 6 mg/kg, followed by a single intravenous administration at a dose of 2 mg/kg. Single subcutaneous administration of the protein of Comparative Example 1 was also performed (five mice) at a dose of 3 mg/kg, respectively, followed by intravenous administration (five mice) at a dose of 1 mg/kg. Thereafter, 0.5 ml of whole blood was collected through the jugular vein according to a predetermined blood sampling schedule [single subcutaneous administration: 0, 0.33, 1, 1.5, 3, 5, 7, 12, 24, 48, 72, 120, and 168 hours (13 points in total), single intravenous administration: 0, 0.083, 0.25, 0.5, 1.25, 3, 5, 10, 24, 48, and 72 hours (11 points in total)], and plasma was separated by centrifugation and stored in a cryogenic freezer (−70° C.). Then, ELISA was performed to quantitatively analyze the protein concentration in the plasma. No dead animals were observed during the experimental period, and no abnormal symptoms related to the administration of the test substance were observed.

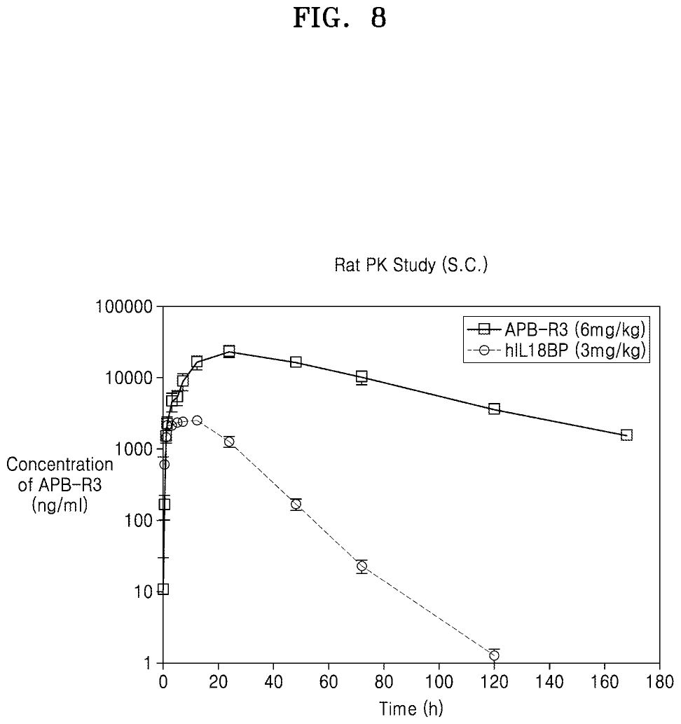

FIG. 8 shows a graph showing protein concentrations in blood after subcutaneous administration of the APB-R3 protein into rats. As shown in FIG. 8, the proteins of the Example 1 and Comparative Example 1 reached the maximum blood concentration (Cₘₐₓ) at 24 hours and 12 hours after subcutaneous administration to rats, respectively, and then decreased. In detail, Example 1 showed 23817.148 ng/mL, and Comparative Example 1 showed 2533.5136 ng/mL, indicating a difference of about 9.4 times. In addition, Example 1 showed in vivo exposure (AUC last) value of 1595699.12 h ng/mL, which was about 22.8 times higher than Comparative Example 1, which showed a value of 69900.47 h·ng/mL. In addition, with regard to an elimination half-life (T_{1/2}), Example 1 showed about 34.92 hours and Comparative Example 1 showed about 9.69 hours, indicating that the half-life of Example 1 was about 3.6 times longer.

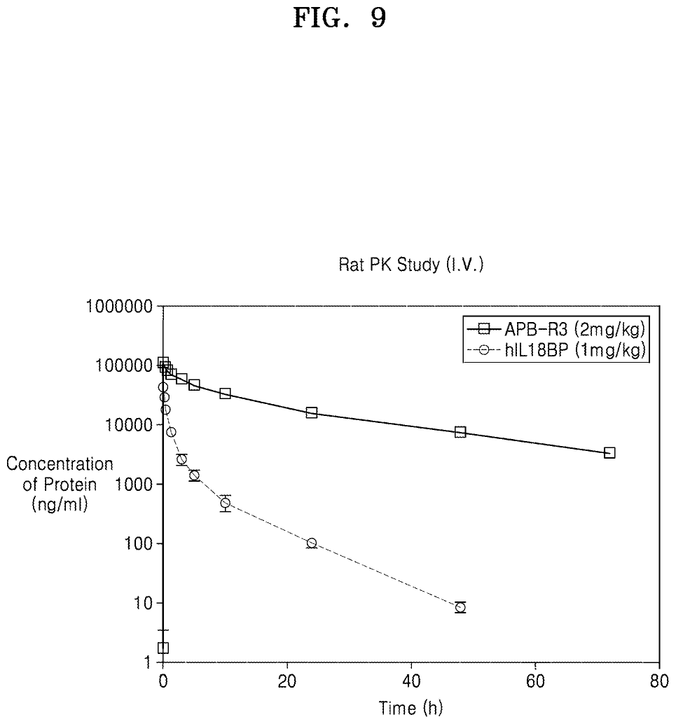

FIG. 9 shows a graph showing protein concentrations in blood after intravenous administration of the APB-R3 protein into rats. As shown in FIG. 9, when measured at 0.083 hours after intravenous administration of Example 1 and Comparative Example 1 into rats, the maximum blood concentration was 109049.3 ng/mL and 41969.6564 ng/mL, respectively, indicating that Example 1 showed about 2.6 times higher concentration. It was also observed that the elimination half-life of Example 1 was 21.81 hours, and the elimination half-life of Comparative Example 1 was 6.51 hours, indicating that the half-life of Example 1 was about 3.4 times longer.

In other words, the recombinant fusion protein can exhibit the increased elimination half-life in the body regardless of the administration method, and therefore, patients can be administered with the drug at more convenient intervals. In addition, as compared with the existing IL-18BP protein, the recombinant fusion protein has the same or similar activity even with a small dose, and thus it is possible to broaden the selection of therapeutic agents.

### Experimental Example 4. In Vivo Efficacy Study in CpG-Induced Macrophage Activation Syndrome Mouse Model

### 4-1. Materials and Methods

All mice used in the experiment were bred in the Animal Laboratory center at Kangwon National University. IL18 bp KO (knock out) mice were established by using CRISPR/Cas9 techniques with C57BL/6 (Orient Bio) by Macrogen (South Korea) and the mice over 8 weeks of age were used in the experiment.

Each of 150 ug of APB-R3, 90 ug of anti-serum albumin Fab antibody (SL335), 250 ug of anti-mouse IL-1beta antibody (BioXcell, BE0246), 250 ug of anti-mouse IL-6R antibody (BioXcell, BE0047), 250 ug of Isotype antibody control (BioXcell, BP0085) and vehicle (PBS) were intraperitoneally administered every day for 9 days. CpG ODN 1826 was synthesized by Integrated DNA Technologies, Inc. (Coralville, Lowa) and dissolved in PBS. After 2 hours of each antibody administration at day 0, 2, 4, 6, 8, 50 ug of dissolved CpG ODN 1826 was intraperitoneally administered in each mouse. During the experiment, all mice were weighed every day with the balance.

Blood samples heparinized by the capillary (Superior, HSU-2901000) in the ophthalmic vein of mice on day 10 were centrifuged at 4° C. to 8000 rpm after incubation for an hour at room temperature. The serum of the upper layer was separated and stored in the cryo-temperature freezer at −80° C. Mouse IFN-gamma ELISA kit (Biolegend, 430804), Mouse CXCL9/MIGA kit (R&D, DY492-05) and Mouse Ferritin ELISA kit (Abcam, ab157713) were used and experiments were conducted according to the manufacturer's recommended protocols. Aspartate transaminase (AST) and alanine transaminase (ALT) were measured by DK Korea (South Korea) using Beckman AU480 (Brea, California) according to IFCC (International Federation of Clinical Chemistry) standard method.

All mice were sacrificed at day 10 and each spleen and liver were extracted for the measurement of weight, cell phenotyping by flow cytometry with BD FACSVerse™ (BD Biosciences), histology by hematoxylin and eosin (H&E) staining.

### 4-2. Results

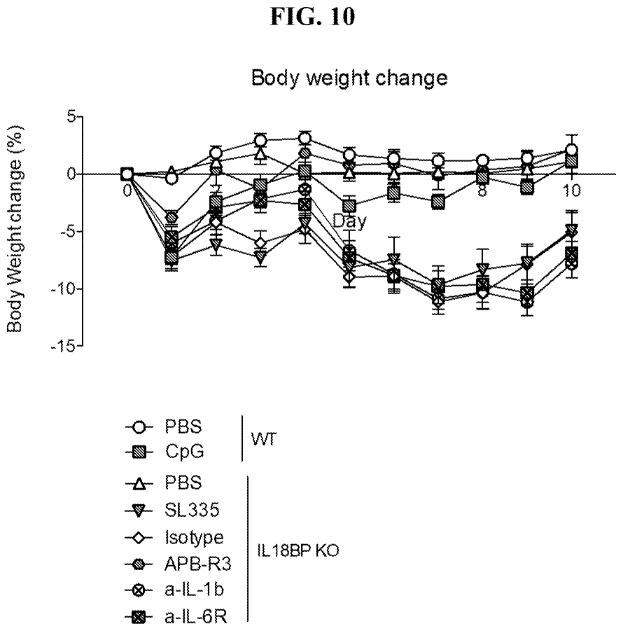

Macrophage activation syndrome (MAS) was induced by CpG, TLR9 agonist, in C57BL/6 mice. A significant loss of weight was observed in every IL18 bp knock-out (KO) mice, but not in wild type (WT) mice. The body weight was similarly recovered in APB-R3 administered KO group and none treated groups, but the group treated with competitive antibodies (anti-TL-6R and anti-IL-1beta) did not recover their weight (FIG. 10).

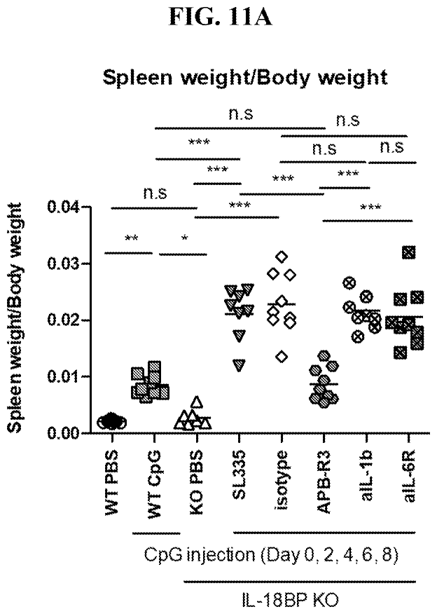

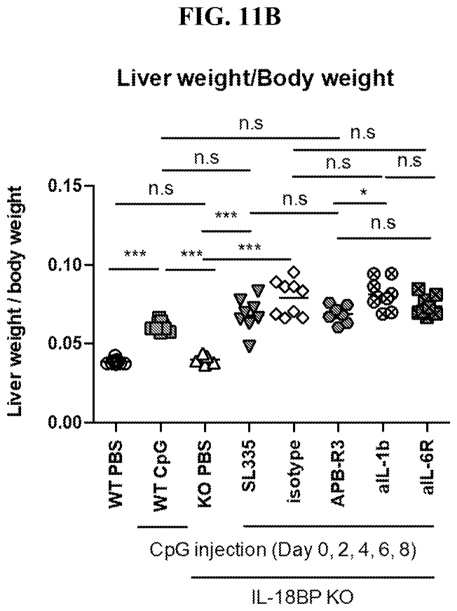

The liver and spleen weight were measured, respectively, at the end of the experiment considering hepatomegaly and splenomegaly of MAS. There was no significant difference of the liver weight/body weight between in CpG+APB-R3 administered KO group and CpG administered WT group (FIG. 11B). On the other hand, the spleen weight/body weight was lower in CpG+APB-R3 administered KO group in comparison to both CpG+anti-IL-6R antibody and CpG+anti-IL-1beta antibody administered KO groups (competitive antibodies) (FIG. 11A).

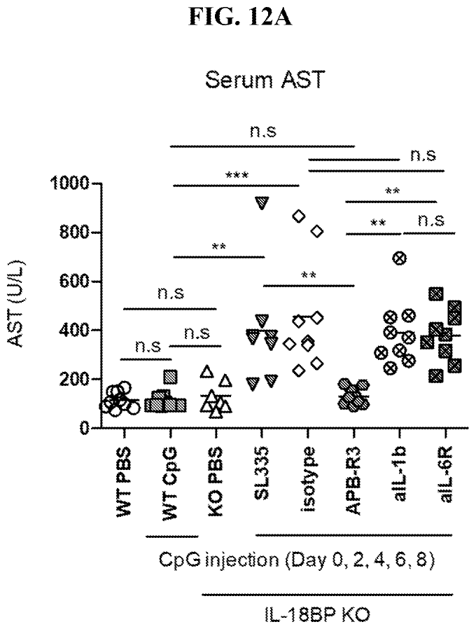

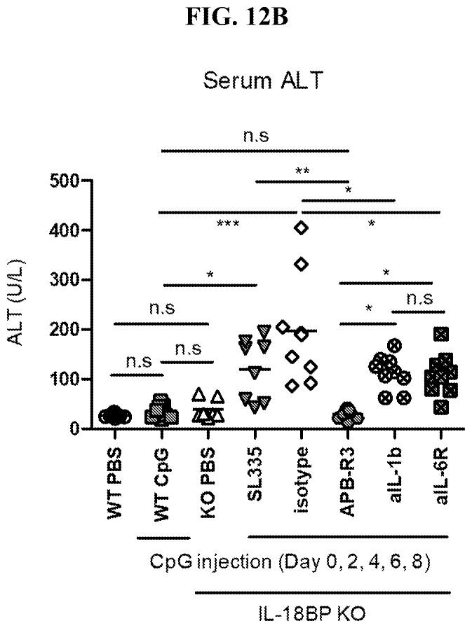

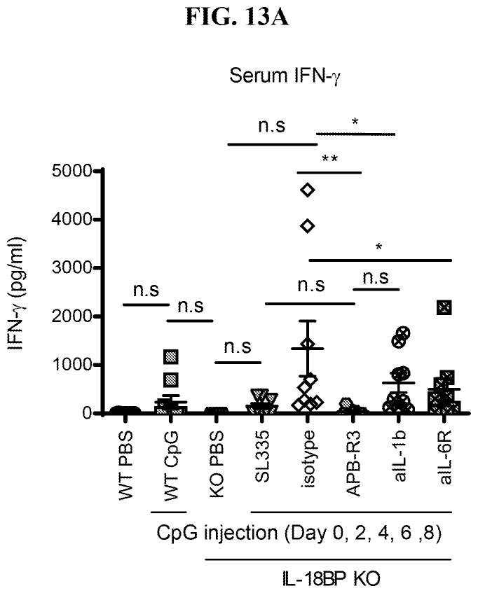

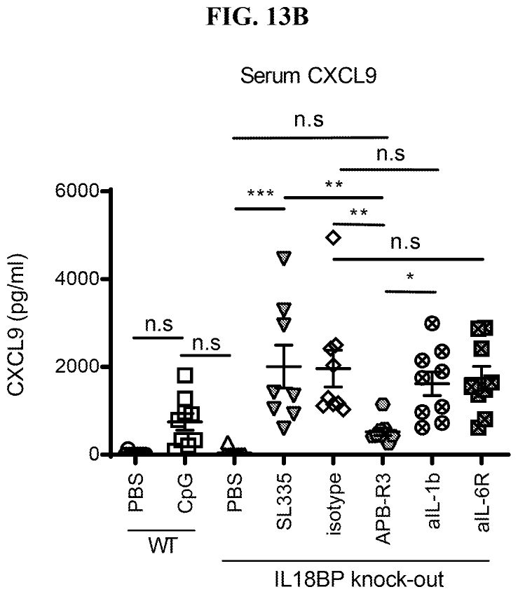

The levels of serum AST and ALT were measured at the end of the experiment with Beckman AU480. The levels of AST and ALT in CpG+APB-R3 administered KO group were similar to none treated WT, none treated KO and CpG administered WT groups, but statistically lower (p<0.05) than that of both CpG+anti-IL-6R antibody and CpG+anti-IL-1beta antibody administered KO groups (FIGS. 12A and 12B). Plus, the levels of serum IFN-γ and CXCL9 were down regulated in CpG+APB-R3 administered KO group compared to other CpG administered groups (FIGS. 13A and 13B).

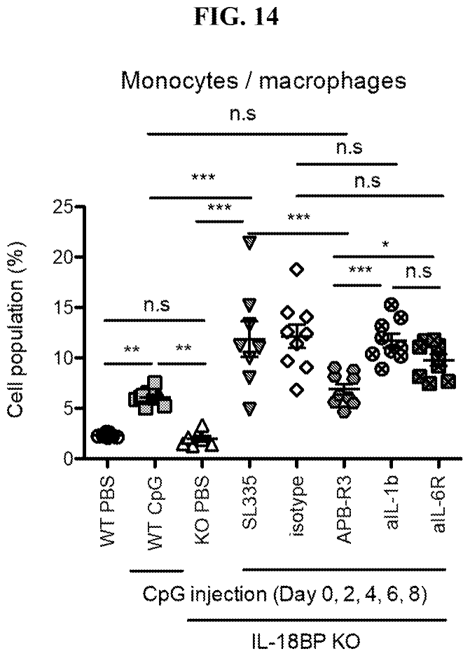

The cell population of splenic monocytes/macrophages was measured by flow cytometry with FACSVerse. The cell population was upregulated in CpG administered groups but slightly lower in APB-R3 administered group which is similar to that of CpG administered WT group (FIG. 14).

The extracted liver and spleen were stained with H&E at the end of the experiment. The disruption of splenic structure was observed in all CpG administered groups but relatively conserved lymphoid follicle area were observed in APB-R3 administered group. Some infiltration of immune cells induced by inflammation were identified in all CpG administered groups, but relatively lower infiltration was observed in APB-R3 administered group in comparison to both competitive antibodies administered groups (data not shown).

### Experimental Example 5. Binding Affinity

### 7-1. Material & Method

Surface Plasmon Resonance (SPR) assay was performed by Wide River Institute of Immunology (Seoul National University, Seoul, KR) to measure the binding affinity, equilibrium dissociation constant (K_{D}), of APB-R3 to recombinant human IL-18 (Prospec) and human serum albumin (Sigma) by using BiaCore™ T200 (GE Healthcare), Sensor Chip CM5 (Cytiva), and HBS-EP+(Cytiva), as a running buffer. APB-R3 was immobilized by using Amine Coupling Kit (GE Healthcare) in pH 4.5 and the analyte of human IL-18 was diluted and prepared at 62.5 nM, 31.3 nM, 15.6 nM, 7.8 nM, 3.9 nM, and 1.95 nM, and the analyte of human serum albumin was diluted and prepared at 1,250 nM, 625 nM, 313 nM, 156 nM, 78 nM, 39 nM, and 19.5 nM. Kinetics between APB-R3 and human IL-18 or human serum albumin was analyzed by 1:1 binding fitting.

### 7-2. Result

The binding affinity (K_{D}) of human IL-18BP isoform a (IL-18BPa) for human IL-18 was known to be 399 pM, as determined by BIAcore affinity assays (Kim, S.-H. et al., Proc. Natl. Acad. Sci. 97:1190-1195 (2000)). In this study, the binding affinity of APB-R3, anti-serum albumin Fab+human IL-18BPa fusion protein to human IL-18 and human serum albumin was determined by Surface Plasmon Resonance (SPR) assay (BiaCore™ T200) and the binding affinity (K_{D}) value of APB-R3 for recombinant human IL-18 was 6.09×10⁻¹¹ M (60.9 pM), which is approximately 6-fold higher than that of human IL-18BPa (60.9 pM vs. 399 pM) as described in Kim S.-H. et al. (2000). The K_{D} value of APB-R3 for human serum albumin was 1.68×10⁻⁸M (16.8 nM) (Table 3).

TABLE 3

| Ligand | Analyte | KD (M) | ka (1/Ms) | kd (1/s) |
|---|---|---|---|---|
| APB-R3 | Human IL-18 | 6.09E−11 | 4.06E+05 | 2.48E−05 |
| | Human serum | 1.68E−08 | 9.38E+04 | 1.57E−03 |
| | albumin | | | |

### Experimental Example 6. In Vitro Immunogenicity Assay of APB-R3

### Material & Methods

In vitro immunogenicity risk of APB-R3 was assessed in a 52 healthy donor human target population by using Lonza's in vitro PBMC proliferation assay platform (Epibase® in vitro proliferation assay) for the analysis of T cell proliferation. A keyhole limpet haemocyanin (KLH) was used as a positive control.

### Results

A significant CD4+ T cell response was induced in 98% (51/52) of donors by KLH positive control and was also significantly different to the blank over the whole test population. A significant CD4+ T cell responses was induced only in 10% (5/52) of donors by APB-R3 and was not significantly different to the blank over the whole test population. The results suggest that APB-R3 can be considered relatively low risk of immunogenicity (Table 4).

TABLE 4

| | Responding | Mean SI (CD4+ | |
|---|---|---|---|
| Molecule | Donors (%) | T cell responses) | p-value |
| APB-R3 | 5/52 (10%) | 1.15 | <0.0581 |
| KLH | 51/52 (98%) | 25.86 | <0.0001 |

*The p-value relates to the single sample t-test hypothesis that the frequency distribution of SI for a given antigen represents a distribution with a mean SI value of 1.

The above description of the present disclosure is only for illustrating, and it will be understood by those skilled in the art that the present disclosure may be easily modified in a different specific form without changing the technical spirit or essential characteristics thereof. Therefore, it should be understood that the above exemplary embodiments are not limitative, but illustrative in all aspects.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be herein incorporated by reference.

What is claimed is:

1. A recombinant fusion protein comprising an interleukin-18-binding protein (IL-18BP) and an antigen binding fragment (Fab) against serum albumin.

2. The protein of claim 1, further comprising a linker that links the IL-18BP to the Fab.

3. The protein of claim 2, wherein the linker links the IL-18BP to a C-terminus of the heavy chain constant domain, an N-terminus of the heavy chain variable domain, a C-terminus of the light chain constant domain, and/or an N-terminus of the light chain variable domain of the Fab.

4. The protein of claim 3, wherein the linker links the IL-18BP to a C-terminus of the heavy chain constant domain.

5. The protein of any one of claims 2-4, wherein the linker comprises 1 to 50 amino acids.

6. The protein of claim 5, wherein the linker comprises an amino acid sequence of any one of SEQ ID NOS:16 and 70-84.

7. The protein of any one of claims 1-6, wherein the heavy chain and the light chain of the Fab are bound by a noncovalent bond.

8. The protein of any one of claims 1-7, wherein the Fab comprises
a heavy chain comprising a heavy chain variable domain comprising
(1) a heavy chain complementarity determining domain 1 (CDR1) comprising the amino acid sequence of SYGIS (SEQ ID NO:22),
a heavy chain complementarity determining domain 2 (CDR2) comprising the amino acid sequence of WINTYSGGTKYAQKFQG (SEQ ID NO:23), and
a heavy chain complementarity determining domain 3 (CDR3) comprising the amino acid sequence of LGHCQRGICSDALDT (SEQ ID NO:24);
(2) a heavy chain CDR1 comprising the amino acid sequence of SYGIS (SEQ ID NO:22),
a heavy chain CDR2 comprising the amino acid sequence of RINTYNGNTGYAQRLQG (SEQ ID NO:25), and
a heavy chain CDR3 comprising the amino acid sequence of LGHCQRGICSDALDT (SEQ ID NO:24);
(3) a heavy chain CDR1 comprising the amino acid sequence of NYGIH (SEQ ID NO:26),
a heavy chain CDR2 comprising the amino acid sequence of SISYDGSNKYYADSVKG (SEQ ID NO:27), and
a heavy chain CDR3 comprising the amino acid sequence of DVHYYGSGSYYNAFDI (SEQ ID NO:28);
(4) a heavy chain CDR1 comprising the amino acid sequence of SYAMS (SEQ ID NO:29),
a heavy chain CDR2 comprising the amino acid sequence of VISHDGGFQYYADSVKG (SEQ ID NO:30), and
a heavy chain CDR3 comprising the amino acid sequence of AGWLRQYGMDV (SEQ ID NO:31);
(5) a heavy chain CDRlcomprising the amino acid sequence of AYWIA (SEQ ID NO:32),
a heavy chain CDR2 comprising the amino acid sequence of MIWPPDADARYSPSFQG (SEQ ID NO:33), and
a heavy chain CDR3 comprising the amino acid sequence of LYSGSYSP (SEQ ID NO:34); or
(6) a heavy chain CDR1 comprising the amino acid sequence of AYSMN (SEQ ID NO:35),
a heavy chain CDR2 comprising the amino acid sequence of SISSSGRYIHYADSVKG (SEQ ID NO:36), and
a heavy chain CDR3 comprising the amino acid sequence of ETVMAGKALDY (SEQ ID NO:37); and
a light chain comprising a light chain variable domain comprising
(7) a light chain CDR1 comprising the amino acid sequence of RASQSISRYLN (SEQ ID NO:38),
a light chain CDR2 comprising the amino acid sequence of GASRLES (SEQ ID NO:39), and
a light chain CDR3 comprising the amino acid sequence of QQSDSVPVT (SEQ ID NO:40);
(8) a light chain CDR1 comprising the amino acid sequence of RASQSISSYLN (SEQ ID NO:41),
a light chain CDR2 comprising the amino acid sequence of AASSLQS (SEQ ID NO:42), and
a light chain CDR3 comprising the amino acid sequence of QQSYSTPPYT (SEQ ID NO:43);
(9) a light chain CDR1 comprising the amino acid sequence of RASQSIFNYVA (SEQ ID NO:44),
a light chain CDR2 comprising the amino acid sequence of DASNRAT (SEQ ID NO:45), and
a light chain CDR3 comprising the amino acid sequence of QQRSKWPPTWT (SEQ ID NO:46);
(10) a light chain CDR1 comprising the amino acid sequence of RASETVSSRQLA (SEQ ID NO:47),
a light chain CDR2 comprising the amino acid sequence of GASSRAT (SEQ ID NO:48), and
a light chain CDR3 comprising the amino acid sequence of QQYGSSPRT (SEQ ID NO:49);
(11) a light chain CDR1 comprising the amino acid sequence ofRASQSVSSSSLA (SEQ ID NO:50),
a light chain CDR2 comprising the amino acid sequence of GASSRAT (SEQ ID NO:48), and
a light chain CDR3 comprising the amino acid sequence of QKYSSYPLT (SEQ ID NO:51); or
(12) a light chain CDR1 comprising the amino acid sequence of RASQSVGSNLA (SEQ ID NO:52),
a light chain CDR2 comprising the amino acid sequence of GASTGAT (SEQ ID NO:53), and
a light chain CDR3 comprising the amino acid sequence of QQYYSFLAKT (SEQ ID NO:54).

9. The protein of any one of claims 1-8,
wherein the heavy chain variable domain comprises a heavy chain CDR1 comprising the amino acid sequence of SEQ ID NO:35, a heavy chain CDR2 comprising the amino acid sequence of SEQ ID NO:36, and a heavy chain CDR3 comprising the amino acid sequence of SEQ ID NO:37, and
wherein the light chain variable domain comprises a light chain CDR1 comprising the amino acid sequence of SEQ ID NO:52, a light chain CDR2 comprising the amino acid sequence of SEQ ID NO:53, and a light chain CDR3 comprising the amino acid sequence of SEQ ID NO:54.

10. The protein of any one of claims 1-9, wherein the heavy chain variable domain comprises an amino acid sequence having at least 90% identity to SEQ ID NO:55, 56, 57, 58, 59, or 60.

11. The protein of any one of claims 1-10, wherein the light chain variable domain comprises an amino acid sequence having at least 90% identity to SEQ ID NO:61, 62, 63, 64, 65, 66, or 67.

12. The protein of any one of claims 1-11, wherein the heavy chain variable domain comprises an amino acid sequence of SEQ ID NO:55, 56, 57, 58, 59, or 60, and the light chain variable domain comprises an amino acid sequence of SEQ ID NO:61, 62, 63, 64, 65, 66, or 67.

13. The protein of any one of claims 1-12, wherein the heavy chain constant domain comprises an amino acid sequence having at least 90% identity to SEQ ID NO:68.

14. The protein of any one of claims 1-13, wherein the light chain constant domain comprises an amino acid sequence having at least 90% identity to SEQ ID NO:69.

15. The protein of any one of claims 1-14, wherein the IL-18-binding protein comprises an amino acid sequence having at least 90% identity to SEQ ID NO:7.

16. The protein of any one of claims 1-15, wherein the IL-18-binding protein comprises an amino acid sequence of SEQ ID NO:7.

17. The protein of any one of claims 1-16, wherein the heavy chain of the Fab comprises an amino acid sequence of SEQ ID NO:19.

18. The protein of any one of claims 1-17 comprising an amino acid sequence of SEQ ID NO:13 and an amino acid sequence of SEQ ID NO:19.

19. A nucleic acid molecule encoding the recombinant fusion protein of any one of claims 1-18.

20. An expression vector comprising the nucleic acid molecule of claim 19.

21. A cell transformed with the expression vector of claim 20.

22. A composition comprising the recombinant fusion protein of any one of claims 1-18.

23. A pharmaceutical composition comprising the composition of claim 22 and a pharmaceutically acceptable excipient.

24. A kit comprising the composition of claim 22 or 23 and a label comprising instructions for a use.

25. A method of treating an immune disease in a subject in need thereof, comprising administering an effective amount of the pharmaceutical composition of claim 23 to the subject.

26. The method of claim 25, wherein the immune disease is an inflammatory disease or autoimmune disease.

27. The method of claim 26, wherein the inflammatory disease is atopic dermatitis, psoriasis, dermatitis, allergy, arthritis, rhinitis, otitis media, sore throat, tonsillitis, cystitis, nephritis, pelvic inflammation, Crohn's disease, ulcerative colitis, ankylosing spondylitis, systemic lupus erythematosus (SLE), asthma, edema, delayed allergy (type IV allergy), transplant rejection, graft-versus-host disease, autoimmune encephalomyelitis, multiple sclerosis, inflammatory bowel disease, cystic fibrosis, diabetic retinopathy, ischemic-reperfusion injury, vascular restenosis, glomerulonephritis, or gastrointestinal allergy.

28. The method of claim 26, wherein the autoimmune disease is adult onset still's disease, systemic juvenile idiopathic arthritis, macrophage activation syndrome, rheumatoid arthritis, Sjogren's syndrome, systemic sclerosis, polymyositis, systemic angitis, mixed connective tissue disease, Crohn's disease, Hashimoto's disease, Grave's disease, Goodpasture's syndrome, Guillain-Barre syndrome, idiopathic thrombocytopenic purpura, irritable bowel syndrome, myasthenia gravis, hypnolepsy, pemphigus vulgaris, pernicious anemia, primary biliary cirrhosis, ulcerative colitis, vasculitis, Wegener's granulomatosis, or psoriasis.

29. A method of treating cancer in a subject in need thereof, comprising administering an effective amount of the pharmaceutical composition of claim 23 to the subject.

30. The method of claim 29, wherein the cancer is multiple myeloma, lung cancer, liver cancer, stomach cancer, colorectal cancer, colon cancer, skin cancer, bladder cancer, prostate cancer, breast cancer, ovarian cancer, cervical cancer, thyroid cancer, kidney cancer, fibrosarcoma, melanoma, or blood cancer.
